(19) United States
(12) Patent Application Publication
(10) Pub. No.: US 2004/0044037 A1
(43) Pub. Date: Mar. 4, 2004

(54) Amidino-urea serotonin receptor ligands and compositions, their pharmaceutical uses, and methods for their snythesis

(76) Inventors: Yufeng Hong, San Diego, CA (US); Atsuo Kuki, Encinitas, CA (US); Eileen Valenzuela Tompkins, Escondido, CA (US); Zhengwei Peng, San Diego, CA (US); David Robert Luthin, Encinitas, CA (US)

Correspondence Address: Mehdi Ganjeizadeh, Warner-Lambert Company, 2800 Plymouth Road, Ann Arbor, MI, 48105, US

(21) Appl. No.: 10/415,619

(22) PCT Filed: Oct. 26, 2001
(86) PCT No.: PCT/IB01/02022

(51) Int. Cl. A61K 31/445; A61K 31/401; A61K 31/165; C07C 279/20

(52) U.S. Cl. 514/317; 514/423; 514/613; 546/226; 548/530; 564/147

(57) ABSTRACT

Novel amidino-urea 5-HT₇ receptor ligands, methods of preparing such ligands, intermediate compounds useful in the preparation of the receptor ligands, pharmaceutical compositions comprising the receptor ligands, and methods of treating sleep disorders, pain, depression, and schizophrenia employing the receptor ligands are disclosed. The receptor ligands have formula (1): wherein the formula variables are as defined herein, and pharmaceutically acceptable salts, solvates, active metabolites, or prodrugs thereof.

### BACKGROUND OF THE INVENTION

1. Field of the Invention

The invention relates to amidino-urea 5-HT₇ receptor ligands, methods of preparing such ligands and intermediates useful in such preparation, and pharmaceutical compositions and treatment methods employing the ligands.

2. Description of the Field of the Invention

The neurotransmitter serotonin (5-hydroxytryptamine, or “5-HT”) has been the subject of substantial research, and abnormalities in serotonin processing are implicated in diverse disease states. Serotonin exerts its effects mainly in the central nervous, cardiovascular, and gastrointestinal systems through binding to a number of discrete 5-HT receptor types, which are assigned to classes and subclasses, e.g., 5-HT₁, 5-HT_{1A}, 5-HT₃, etc., based on their pharmacological properties such as ligand binding profiles, coupling to second messenger systems, functional activity, and protein structures. The properties, functions, and pharmacology of these receptor subtypes have been reviewed by (a) Kennett, G. A., “Serotonin Receptors and Their Function,” TOCRIS Review (htt://www.tocris.com/serotonin.htm), published May, 1997; (b) Peroutka, S. J., 1994, “Molecular Biology of Serotonin (5-HT) Receptors, Synapse 18, 241-260; and (c) Eglen, R. et al., 1997, “The 5-HT₇ Receptor: Orphan Found, TiPs, April 1997 (Vol. 18), pp. 104-107.

While the 5-HT₃ receptor forms a ligand-gated ion channel, most of the other serotonin receptor types are linked to increases or decreases of cyclic AMP production. Receptors of the 5-HT₁ family are negatively coupled to adenylyl cyclase through guanine-nucleotide-binding (G) proteins; those of the 5-HT₂ family stimulate phospholipase C. The 5-HT₄, 5HT₆, and 5HT₇ receptors stimulate adenylyl cyclase via Gₛ coupling. Cloning and function of these receptor types are reviewed by Lucas, J. J. and Hen, R, 1995, “New Players in the 5-HT Receptor Field: Genes and Knockouts,” TiPS, July, 1995 (Vol. 16) pp. 246-252.

The 5-HT₇ receptors form a distinct family of G-protein coupled receptors positively coupled to adenylyl cyclase. The 5-HT₇ receptor has been cloned from rat, mouse, guinea pig, and human cDNA. Despite a high degree of inter-species homology (95%), the receptor has low homology (<40%) with other 5-HT receptor subtypes. The pharmacological profile of the receptor is also consistent across species and is characterized by a high affinity for the 5-HT₁ agonists, 5-carboxyamidotryptamine (5-CT), 5-HT, and 5-methoxytiyptamine.

5-HT₇ receptors are expressed in hypothalamus, hippocampus, thalamus, and other limbic areas and may be involved in regulation of circadian rhythms. 5-HT₇ receptors have high affinity for certain antidepressant and antipsychotic drugs, including pimozide, an antipsychotic used to treat Tourette syndrome, and the atypical antipsychotic drug, clozapine. Biochemical and pharmacologic studies have pointed to the role of 5-HT in the following conditions:

depression (Sleight, A. J., et al., 1995, “Identification of 5-Hydroxytryptamine₇ Receptor Binding Sites in Rat Hypothalamus: Sensitivity to Chronic Antidepressant Treatment,” Molecular Pharmacol. 47:99-103; Shimizu, M. et al., 1996, “Chronic Antidepressant Exposure Enhances 5-Hydroxytryptamine₇ Receptor-Mediated Cyclic Adenosine Monophosphate Accumulation in Rat Frontocortical Astrocytes,” J. Pharmacol. Exper. Therapeutics 279:1551-1558);

psychosis (Roth, B. L. et al., 1994, “Binding of Typical and Atypical Antipsychotic Agents to 5-Hydroxytryptamine-6 and 5-Hydroxytryptamine-7 Receptors,” J. Pharmacol. Exper. Therapeutics 268: 1403-1410);

cardiovascular disease (Cushing, D. J. et al., 1996, “LY215840, a High-Affinity 5HT₇ Receptor Ligand, Blocks Serotonin-induced Relaxation in Canine Coronary Aritery,” J. Pharmacol. Exper. Ther. 277:1560-1566; Terron, J., 1998, “The Relaxant 5-HT HT Receptor in the Dog Coronary Artery Smooth Muscle: Pharmacological Resemblance to the Cloned 5-ht₇ Receptor Subtype,” Proc. West. Pharmacol. Soc. 41:129-30); and

affective behaviors and modulation of sensory information (To, Z. et al., 1995, “Characterization and Distribution of Putative 5-ht₇ Receptors in Guinea Pig Brains,” Brit. J. Pharmacol. 115:107-116).

At present, very few selective ligands for 5-HT₇ receptors have been reported (Forbes, I. T. et al., “(R)-3-N-Dimethyl-N[1-methyl-3(4-methyl-piperidin-1yl)propyl]benzene-sulfonamide: The First Selective 5-HT₇ Receptor Antagonist,” J. Med. Chem. 41, 655-657 (1998); Kikuchi et al., “Tetrahydrobenzindoles: Selective Antagonists of the 5-HT₇ Receptor,” J. Med. Chem. 42, 533-535 (1999); Lovell et al., “A Novel Potent, and Selective 5-HT₇ Antagonist: (R)-3-(2-(2-(4-Methylpiperidinyl-1-yl)-ethyl)pyrrolidine-1-sulfonyl)phenol (SB-269970),” J. Med. Chem. 43, 342-345, (2000); “Functional Characteristics of the Human Cloned 5-HT₇ Receptor (long form) Antagonist Profile of SB-258719,” British J. Pharm, 124, 1300-1306 (1998); Prous Science (abstracts) of Asai et al., 72^{nd} Annual Meet Jpn. Pharmacol. Soc. (March 23-25, Sapporo), 1999—Abst. P-496, Needham et al., Eur. Neuropsychopharmacol. [12^{th} Cong. Eur. Coll Neuropsychopharmacol. (September 21-25, London)] 1999, 9, (Suppl.5)—Abst. P.2.021; WO 99/31062 and WO/00/0472).

The 5-HT₇ receptor may be involved in the pathophysiology of sleep disorders, depression, pain, and schizophrenia. Potent and selective ligands active at 5-HT₇ receptors are needed to provide novel pharmaceutical approaches to treatment of these disorders.

### SUMMARY OF THE INVENTION

This invention is directed to compounds represented by the formula:

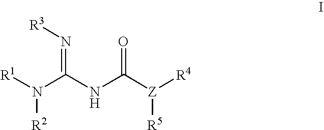

wherein:

Z is N, O or CH;

R¹ is H or lower alkyl;

R² is alkyl, cycloalkyl, arylalkyl or heteroarylalkyl, wherein the alkyl, cycloalkyl, aryl and heteroaryl moieties thereof may be substituted or unsubstituted; or

R¹ and R² together with the nitrogen to which they are bound form a 5- or 6membered ring, which may be substituted or unsubstituted;

R³ is H, lower alkyl or lower alkylaminocarbonyl;

R⁴ is H, alkyl, alkenyl, arylalkyl, heteroarylalkyl, heterocycloalkylalkyl, arylalkenyl, heteroarylalkenyl, cycloalkyl, cycloalkenyl, heterocycloalkyl, aryl or heteroaryl, wherein the alkyl, alkenyl, cycloalkyl, cycloalkenyl, heterocycloalkyl, aryl and heteroaryl moieties thereof may be substituted or unsubstituted; or

R⁵ is absent (when Z is O) or is H or lower alkyl; or

R⁴ and R⁵ together with Z form a 5- or 6-membered ring, which may be substituted or unsubstituted;

and pharmaceutically acceptable salts, solvates, active metabolites, or prodrugs thereof.

These compounds are potent antagonists for 5-HT₇ receptors and show selectivity for 5-HT₇ receptors over other serotonin receptor subtypes and over other receptors such as D₂ dopamine, α₁ adrenergic (α_{1A}, α_{1B}, α_{1D}), α₂ adrenergic (α_{2A}, α_{2B}, α_{2C)}, hGalanin, opiate (δ, μ, κ), GABA-B, and muscarinic (M₁, M₂, M₃, M₄, M₅). The compounds have potential utility in the treatment of pain, depression, sleep disorders, and schizophrenia.

The invention also encompasses pharmaceutically acceptable salts, solvates, active metabolites, or prodrugs comprising the compounds of Formula I, and includes pharmaceutical compositions comprising the compounds of Formula I as well as pharmaceutically acceptable salts, solvates, active metabolites, or prodrugs thereof. The invention is also related to a method of treatment of a patient in need thereof with a pharmaceutical composition comprising an effective amount of a compound of Formula I, or a pharmaceutically acceptable salt, solvate, active metabolite, or prodrug thereof.

The invention is also directed to methods of preparation of the compounds represented by Formula I. The invention also comprises intermediates and pharmaceutically acceptable salts thereof, useful in the synthesis of compounds of Formula I.

### DETAILED DESCRIPTION OF THE INVENTION

In accordance with a convention used in the art,

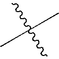

is used in structural formulas herein to depict the bond that is the point of attachment of the moiety or substituent to the core or backbone structure.

As used herein, the term “alkyl” represents a straight- or branched-chain saturated hydrocarbon group, containing 1 to 20 carbon atoms, which may be unsubstituted or substituted by one or more of the substituents described below. Exemplary alkyl groups include, but are not limited to methyl (Me), ethyl (Et), propyl, isopropyl, butyl, isobutyl, t-butyl, and the like. The term Alower alkyl@ refers to an alkyl group having from 1 to 6 carbon atoms in its chain.

“Alkenyl” represents a straight- or branched-chain hydrocarbon group, containing 1 to 10 carbon atoms and one or more carbon-carbon double bonds, and which may be unsubstituted or substituted by one or more of the substituents described below. Exemplary alkenyl groups include, but are not limited to, ethenyl, propenyl, butenyl, butadienyl, isobutenyl, and the like

“Cycloalkyl” represents a group comprising a saturated monocyclic, bicyclic, or tricyclic hydrocarbon containing from 3 to 14 carbon atoms that may be a mono- or poly-carbocyclic ring, preferably having 5-14 ring carbon atoms. Exemplary cycloalkyl groups include monocyclic rings having from 3-7, preferably 3-6, carbon atoms, such as cyclopropyl, cyclobutyl, cyclopentyl, cyclohexyl, cycloheptyl and the like. Exemplary bicyclic and tricyclic cycloalkyls include groups having from 10-14 carbon atoms. Illustrative examples of cycloalkyl groups include the following:

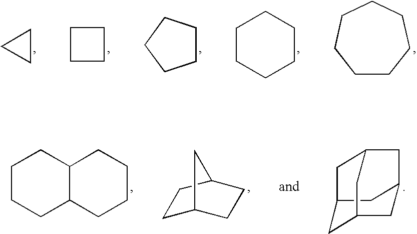

“Cycloalkenyl” represents a group comprising a non-aromatic carbocycle containing from 5 to 14 ring carbon atoms that may be a mono- or poly-carbocyclic ring, to which may be fused an aryl moiety. Exemplary cycloalkenyl groups include monocyclic groups having from 5-8 carbon atoms or bi- or tricyclic groups having from 9-14 carbon atoms, such as cyclopentenyl, cyclopentadienyl, tetrahydronaphthalene, dihydroindenyl, cyclohexenyl, cycloheptenyl and the like. Illustrative examples of cycloalkenyl groups include the following:

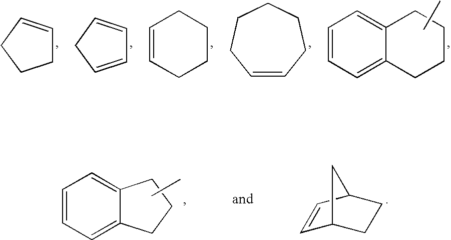

“Heterocycloalkyl” represents a group comprising a non-aromatic, monovalent monocyclic, bicyclic, or tricyclic radical, which is saturated or partially unsaturated, containing 3 to 18 ring atoms, which includes 1 to 5 heteroatoms selected from nitrogen, oxygen and sulfur, and which may be unsubstituted or substituted by one or more of the substituents described below. Illustrative examples of heterocycloalkyl groups include, but are not limited to, azetidinyl, pyrrolidyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl, 1,5,9-triazacyclododecyl, and the like. Illustrative examples of heterocycloalkyl groups include the following moieties:

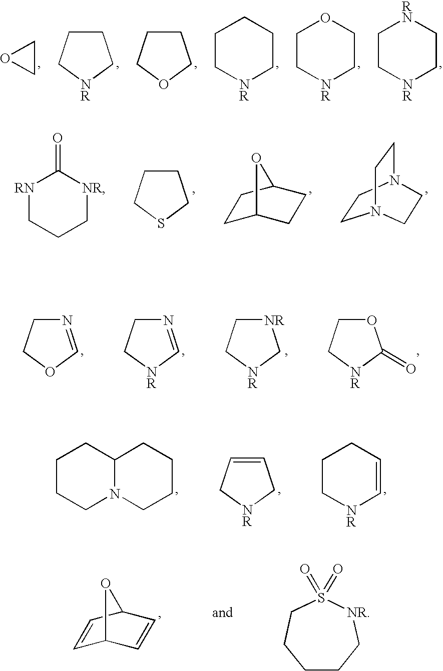

“Aryl@ represents a group comprising an aromatic, monovalent monocyclic, bicyclic, or tricyclic radical containing from 6 to 18 carbon ring atoms, which may be unsubstituted or substituted by one or more of the substituents described below. Illustrative examples of aryl groups include the following:

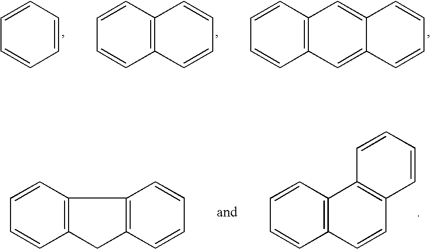

“Heteroaryl@ represents a group comprising an aromatic monovalent monocyclic, bicyclic, or tricyclic radical, containing 5 to 18 ring atoms, including 1 to 5 heteroatoms selected from nitrogen, oxygen and sulfur, which may be unsubstituted or substituted by one or more of the substituents described below. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenantlridinyl, acridinyl, perimidinyl, phenanthrolinyl, phenazinyl, isothiazolyl, phenothiazinyl, and phenoxazinyl. Further examples of heteroaryl groups include the following moieties:

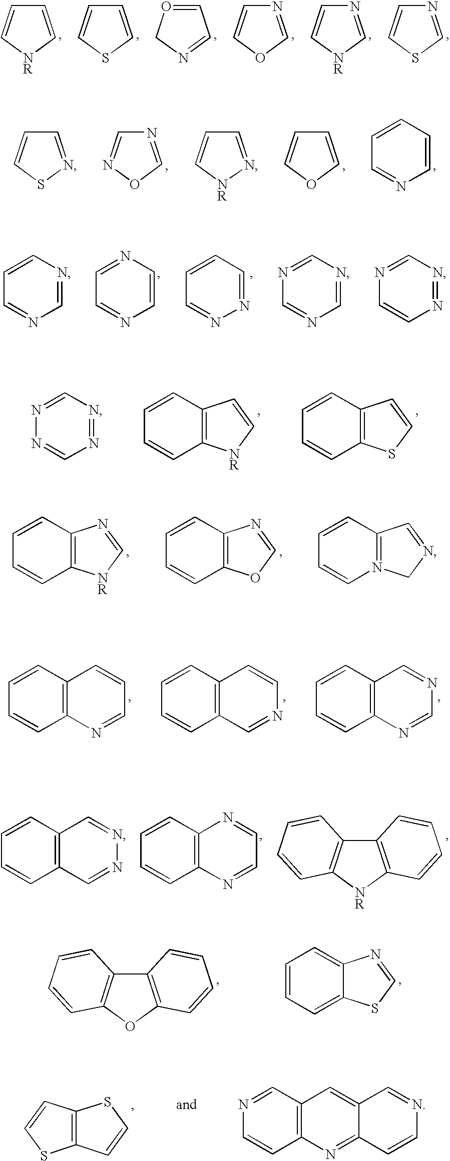

As indicated herein, the alkyl, alkenyl, cycloalkyl, cycloalkenyl, aryl, heterocycloalkyl and heteroaryl groups may be optionally substituted by one or more substituents. The term “optionally substituted” is intended to expressly indicate that the specified group is unsubstituted or substituted by one or more suitable substituents. The term “substituent” or “suitable substituent” is intended to mean any suitable substituent that may be recognized or selected, such as through routine testing, by those skilled in the art.

Exemplary substituents that may be present on any of the above alkyl, alkenyl, cycloalkyl, cycloalkenyl, aryl, heterocycloalkyl and heteroaryl groups are described herein and include alkyl, arylalkyl, cycloalkylalkyl, heterocycloalkylalkyl, heteroarylalkyl, aryl, cycloalkyl, heterocycloalkyl, heteroaryl, nitro, amino, cyano, halo, hydroxyl, alkylhydroxyl,alkoxy, alkylenedioxy, aryloxy, cycloalkoxy, heterocycloalkoxy, heteroaryloxy, alkylcarbonyl, alkyloxycarbonyl, alkylcarbonyloxy, arylcarbonyl, arylcarbonyloxy, aryloxycarbonyl, cycloalkylcarbonyl, cycloalkylcarbonyloxy, cycloalkyoxycarbonyl, heteroarylcarbonyl, heteroarylcarbonyloxy, heteroaryloxycarbonyl, heterocycloalkylcarbonyl, heterocycloalkylcarbonyloxy, heterocycloalkyoxycarbonyl, carboxyl, carbamoyl, formyl, keto (oxo), thioketo, sulfo, aminoalkyl, alkylamino, cycloalkylamino, arylamino, heterocycloalkylamino, heteroarylamino, dialkylamino, alkylaminocarbonyl, cycloalkylaminocarbonyl, arylaminocarbonyl, heterocycloalkylaminocarbonyl, heteroarylaminocarbonyl, dialkylaminocarbonyl, alkylaminothiocarbonyl, cycloalkylaminothiocarbonyl, arylaminothiocarbonyl, heterocycloalkylaminothiocarbonyl, heteroarylaminothiocarbonyl, dialkylaminothiocarbonyl, alkylsulfonyl, arylsulfonyl, alkylsulfenyl, arylsulfenyl, alkylcarbonylamino, cycloalkylcarbonylamino, arylcarbonylamino, heterocycloalkylcarbonylamino, heteroarylcarbonylamino, alkylthiocarbonylamino, cycloalkylthiocarbonylamino, arylthiocarbonylamino, heterocycloalkylthiocarbonylamino, heteroarylthiocarbonylamino, alkylsulfonyloxy, arylsulfonyloxy, alkylsulfonylamino, arylsulfonylamino, mercapto, alkylthio, arylthio, heteroarylthio, wherein any of the alkyl, alkylene, aryl, cycloalkyl, heterocycloalkyl,. heteroaryl moieties present in the above substituents may be further substituted. The alkyl, alkylene, cycloalkyl, heterocycloalkyl, aryl, and heteroaryl moieties of any of the above substituents may be optionally substituted by one or more of alkyl, haloalkyl, aminoalkyl, arylalkyl, cycloalkylalkyl, heterocycloalkylalkyl, heteroarylalkyl, substituted or unsubstituted aryl, nitro, cyano, amino, alkylamino, arylamino, dialkylamino, halo, hydroxyl, alkoxy, haloalkoxy, substituted or unsubstituted aryloxy, alkylcarbonylamino, alkylaminocarbonyl, alkylenedioxy, alkylcarbonyl, aminocarbonyl, alkoxycarbonyl, arylcarbonyl, mercapto, alkylthio or substituted or unsubstituted arylthio groups, wherein the aryl moieties of the above substituents may be further substituted by alkyl, haloalkyl, nitro, amino, alkylamino, dialkylamino, halo, hydroxyl, alkoxy, haloalkoxy, aryloxy, mercapto, alkylthio or arylthio groups.

Preferred substituents in the compounds of this invention include one or more of: lower alkyl, aryl, arylalkyl, cycloalkylalkyl, heterocycloalkylalkyl, heteroarylalkyl, cycloalkyl, heterocycloalkyl, heteroaryl, halo, hydroxyl, alkoxy, haloalkoxy, aryloxy, cycloalkoxy, heteroaryloxy, nitro, cyano, amino, alkylamino, arylamino, dialkylamino, aminocarbonyl, alkylaminocarbonyl, alkylenedioxy, alkylcarbonyl, alkylcarbonylarrno, alkoxycarbonyl, arylcarbonyl, aryloxycarbonyl, mercapto, alkylthio or arylthio, wherein any of the alkyl, cycloalcyl and heterocycloalkyl moieties thereof may be optionally substituted by one or more of alkyl, haloalkyl, aminoalkyl, aldylhydroxyl, arylalkyl, cycloalkylalkyl, heterocycloalkylalkyl, heteroarylalkyl, aryl, heteroaryl, heterocycloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, aryloxycarbonyl,.mercapto, alkylthio or arylthio, wherein the aryl or heteroaryl moieties of any of the above substituents may be further substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups.

The terms “halogen” and “halo” represent chloro, fluoro, bromo or iodo substituents. AHeterocycle@ is intended to mean a heteroaryl or heterocycloalkyl group. “Acyl@ is intended to mean a —C(O)—R radical, wherein R is an alkyl, cycloalkyl, aryl, heterocycloalkyl or heteroaryl group. “Acyloxy@ is intended to mean an —OC(O)—R radical, wherein R is an alkyl, cycloalkyl, aryl, heterocycloalkyl or heteroaryl group. AThioacyl@ is intended to mean a —C(S)—R radical, wherein R is an alkyl, cycloalkyl, aryl, heterocycloalkyl or heteroaryl group. ASulfonyl@ is intended to mean an —SO₂— biradical. ASulfenyl@ is intended to mean an —SO— biradical. ASulfo@ is intended to mean an —SO₂H radical. AHydroxyl@ or “hydroxy” are intended to mean the radical —OH. AAmine@ or Aamio is intended to mean the radical —NH₂. AAlkylamino@ is intended to mean the radical —NHₐ, wherein Rₐ is an alkyl group. AAminoalkyl@ is intended to mean the radical —RₐNH₂, wherein Rₐ is an alkyl group. AArylamino@ is intended to mean the radical —NHRₐ, wherein Rₐ is an aryl group. ADialkylamino@ is intended to mean the radical —NRₐR_{b}, wherein Rₐ and R_{b} are each independently an alkyl group, and is intended to include heterocycloalkyl groups, wherein Rₐ and R_{b} , taken together, form a heterocyclic ring that includes the amine nitrogen. “Alkylhydroxyl” is intended to mean the radical —RₐOH, wherein Rₐ is an alkyl group. AAlkoxy@ is intended to mean the radical —ORₐ, wherein Rₐ is an alkyl group. Exemplary alkoxy groups include methoxy, ethoxy, propoxy, and the like. ALower alkoxy@ groups have alkyl moieties having from 1 to 4 carbons. AALkylenedioxy@ is intended to mean the divalent radical —ORₐO— which is bonded to adjacent atoms (e.g., adjacent atoms on a phenyl or naphthyl ring) , wherein Rₐ is a lower alkyl group. AAlkoxycarbonyl@ or “alkyloxycarbonyl” are intended to mean the radical —C(O)ORₐ, wherein Rₐ is an alkyl group. AAlkylsulfonyl@ is intended to mean the radical —SO₂Rₐ, wherein Rₐ is an alkyl group. “Alkylaminocarbonyl” is intended to mean the radical —C(O)NHRₐ, wherein Rₐ is an alkyl group. ADialkylaminocarbonyl” is intended to mean the radical —C(O)NHₐR_{b}, wherein Rₐ and R_{b} are each independently an alkyl group. “Mercapto” is intended to mean the radical —SH. “Alkylthio” is intended to mean the radical —SRₐ, wherein Rₐ is an alkyl group. “Carboxyl” is intended to mean the radical —C(O)OH. AKeto@ or Aoxo@ is intended to mean the radical ═O. AThioketo@ is intended to mean the radical ═S. “Carbamoyl” is intended to mean the radical —C(O)NH₂. ACycloalkylalkyl@ is intended to mean the radical Balkyl-cycloalkyl, wherein alkyl and cycloalkyl are defined as above, and is represented by the bonding arrangement present in the groups —CH₂-cyclohexane or —CH₂-cyclohexene. AArylalkyl— is intended to mean the radical Balkylaryl, wherein the alkyl and aryl moieties thereof are defined as above (e.g., wherein “alkyl” represents a straight- or branched-chain saturated hydrocarbon group, containing 1 to 20 carbon atoms, which may be unsubstituted or substituted by one or more substituents) and is represented by the bonding arrangement present in a benzyl group. “Heteroarylalkyl” is intended to mean the radical Balkyl-heteroaryl, wherein the alkyl and heteroaryl moieties thereof are defined as above and is represented by the bonding arrangement present in an α-methylfuranyl group. “Heterocycloalkylalkyl” is intended to mean the radical Balkyl-heterocycloalkyl, wherein the alkyl and heterocycloalkyl moieties thereof are defined as above and is represented by the bonding arrangement present in an α-methylpiperidinyl group. “Cycloalkylalkyl” is intended to mean the radical Balkyl-cycloalkyl, wherein the alkyl and cycloalkyl moieties thereof are defined as above and is represented by the bonding arrangement present in an α-methylcyclohexyl group. AAminocarbonylalkyl@ is intended to mean the radical BalkylC(O) NH₂ and is represented by the bonding arrangement present in the group —CH₂CH₂C(O)NH₂. AAlkylaminocarbonylalkyl@ is intended to mean the radical BalkylC(O)NHRₐ, wherein Rₐ is an alkyl group and is represented by the bonding arrangement present in the group —CH₂CH₂C(O)NHCH₃. AAlkylcarbonylaminoalkyl is intended to mean the radical BalkylNHC(O)-alkyl and is represented by the bonding arrangement present in the group —CH₂NHC(O)CH₃. ADialkylaminocarbonylalkyl@ is intended to mean the radical BalkylC(O)NRₐR_{b}, wherein Rₐ and R_{b} are each independently an alkyl group. “Aryloxy” is intended to mean the radical —OR_{c}, wherein R_{c} is an aryl group. “Heteroaryloxy” is intended to mean the radical —OR_{d}, wherein R_{d} is a heteroaryl group. “Arylthio” is intended to mean the radical —SR_{c}, wherein R_{c} is an aryl group. “Heteroarylthio” is intended to mean the radical —SR_{d}, wherein R_{d} is a heteroaryl group.

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum or lithium.

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid.

The inventive compounds may exist as single stereoisomers and/or diastereomers, racemates, and/or mixtures of enantiomers and/or diastereomers. All such single stereoisomers, diastereomers, racemates, and mixtures thereof are intended to be encompassed within the broad scope of the present invention. Where the stereochemistry of the chiral carbons present in the chemical structures illustrated herein is not specified, the chemical structure is intended to encompass compounds containing either stereoisomer of each chiral carbon. Preferably, however, the inventive compounds are used in optically pure form. When used describe a particular compound, the term “optically pure” is used herein to that the compound is substantially enantiomerically or diastereomerically pure. Compounds that are substantially enatiomerically pure contain at least 90% of a single isomer and preferably contain at least 95% of a single isomer. Compounds that are substantially diastereomerically pure contain at least 90% of a single isomer of each chiral carbon center present in the diastereomer, and preferably contain at least 95% of a single isomer of each chiral carbon. More preferably, the optically active compounds in this invention contain at least 97.5% of a single isomer and most preferably contain at least 99% of a single isomer. Compounds identified herein as single stereoisomers are meant to describe compounds that are present in a form that contains at least 90% of a single isomer. The term Aracemic@ or Aracemic mixture@ refers to a mixture of equal amounts of enantiomeric compounds, which encompasses mixtures of enantiomers and mixtures of enantiomeric diastereomers.

The compounds of the invention described herein may also exhibit the phenomenon of tautomerism. The structural formulae herein depict one of the possible tautomeric forms, but it should be understood that the invention nonetheless encompasses all tautomeric forms of the compounds.

Preferred embodiments of the compounds of this invention are represented by the formulas:

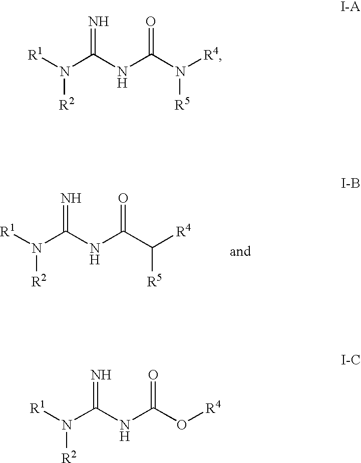

wherein R¹, R², R³, R⁴ and R⁵ are as defined above, and include the pharmaceutically acceptable salts, solvates, active metabolites, or prodrugs thereof.

In the compounds of this invention, where R² is substituted alkyl, cycloalkyl, arylalkyl or heteroarylalkyl, the alkyl, cycloalkyl, aryl or heteroaryl moieties of these R² substituents may be substituted by one or more substituents independently selected from alkyl, aryl, heteroaryl, halo, hydroxyl, alkoxy, haloalkoxy, aryloxy, cycloalkoxy, heteroaryloxy, nitro, cyano, amino, alkylamino, arylamino, dialkylamino, aminocarbonyl, alkylaminocarbonyl, alkylenedioxy, alkylcarbonyl, alkylcarbonylamino, alkoxycarbonyl, arylcarbonyl, aryloxycarbonyl, mercapto, alkylthio or arylthio. Where R¹ and R² together with the nitrogen to which they are both bound form a 5- or 6-membered ring, the ring may be substituted with one or more substituents independently selected from alkyl, aryl, heteroaryl, halo, hydroxyl, alkoxy, haloalkoxy, aryloxy, cycloalkoxy, heteroaryloxy, nitro, cyano, amino, alkylamino, arylamino, dialkylamino, aminocarbonyl, alkylaminocarbonyl, alkylcarbonyl, alkylcarbonylamino, alkoxycarbonyl, arylcarbonyl, aryloxycarbonyl, mercapto, alkylthio or arylthio. In addition, the aryl moieties of any of the above substituents may be further substituted by alkyl, haloalkyl, halo, hydroxyl, aryl, lower alkoxy, aryloxy, amino, nitro, cyano or haloalkoxy groups. Preferably, the alkyl, cycloalkyl, aryl or heteroaryl moieties of R² or the ring formed by R¹ and R² may be substituted by hydroxyl, halo, alkyl, aryl, arylalkyl, (di-aryl)alkyl, lower alkoxy and aryloxy.

In the compounds of this invention, where R⁴ is substituted alkyl, arylalkyl, heteroarylalkyl, heterocycloalkylalkyl, arylalkenyl, heteroarylalkenyl, cycloalkyl, cycloalkenyl, heterocycloalkyl, aryl or heteroaryl, the alkyl, alkenyl, cycloalkyl, heterocycloalkyl, aryl or heteroaryl moieties of these R⁴ substituents may be substituted by one or more substituents independently selected from alkyl, haloalkyl, aminoalkyl, arylalkyl, cycloalkylalkyl, heterocycloalkylalkyl, aryl, heteroaryl, heterocycloalkyl, nitro, cyano, amino, alkylamino, arylamino, dialkylamino, halo, hydroxyl, alkylhydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, aryloxycarbonyl, mercapto, alkylthio, arylthio groups or may be substituted by a spiro, fused or spiro-fused cycloalkyl or heterocycloalkyl group which may be unsubstituted or substituted by alkyl, haloalkyl, aminoalkyl, arylalkyl, aryl, heteroaryl, alkylamino, arylamino, dialkylamino, halo, hydroxyl, alkylhydroxyl, aryloxy, alkoxy, keto (oxo), alkylcarbonylamino, alkylaminocarbonyl, alkylcarbonyl, aminocarbonyl, alkoxycarbonyl, arylcarbonyl or aryloxycarbonyl, wherein the aryl or heteroaryl moieties of any of the above substituents may be further substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups.

In the compounds of this invention, where R⁴ and R⁵ together with the atom to which they are attached form a 5- or 6-membered ring, this ring may be substituted with one or more substituents independently selected from substituted or unsubstituted lower alkyl, cycloalkenyl, heteroaryl, heterocycloalkyl, arylalkyl, arylalkenyl, heteroarylalkyl, heterocycloalkylalkyl, aryl, nitro, cyano, amino, alkylamino, arylamino, dialkylamino, halo, keto (oxo), hydroxyalkyl, hydroxyl, alkoxy, alkylenedioxy, haloalkoxy, aryloxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, arylcarbonyl, mercapto, alkylthio or arylthio groups, wherein the alkyl, alkenyl, cycloalkyl, heterocycloalkyl, aryl or heteroaryl moieties thereof may be substituted by one or more substituents independently selected from alkyl, haloalkyl, aminoalkyl, alkylhydroxyl, aryl, nitro, cyano, amino, alkylamino, arylamino, dialkylamino, halo, hydroxyl, alkoxy, alkylenedioxy, haloalkoxy, aryloxy, alkylcarbonylamino, alkylaminocarbonyl, alkylcarbonyl, aminocarbonyl, alkoxycarbonyl, arylcarbonyl, mercapto, alkylthio or arylthio groups, wherein the aryl moieties of any of the above substituents may be further substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups;

In preferred embodiments of the compounds of this invention, R¹ and R³ are hydrogen and R² is arylalkyl. More preferably, R² is aryl-methylene and the compounds of this embodiment have the formula:

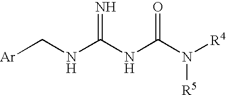

where R⁴ and R⁵ are as defined above.

In especially preferred embodiments, R² is naphthylmethyl and the compounds of this embodimentmay be represented by the formula:

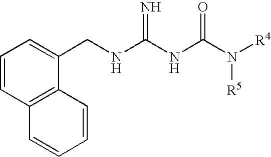

where R⁴ and R⁵ are as defined above. Preferably, R⁵ is H and R⁴ is as defined above.

In another embodiment of the compounds of this invention R⁴ and R⁵ form a 6membered member ring and have the formula:

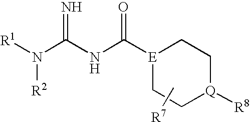

wherein R¹ and R² are as defined above;

E is N or CH;

Q is N or CH;

R⁷ and R⁸ are independently selected from H, substituted or unsubstituted lower alkyl, cycloalkenyl, heteroaryl, heterocycloalkyl, arylalkyl, arylalkenyl, heteroarylalkyl, heterocycloalkylalkyl, aryl, cyano, amino, alkylamino, arylamino, dialkylamino, keto (oxo), hydroxyalkyl, hydroxyl, alkoxy, alkylenedioxy, haloalkoxy, aryloxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, arylcarbonyl, alkylthio or arylthio groups, wherein the alkyl, alkenyl, cycloalkyl, heterocycloalkyl, aryl or heteroaryl moieties thereof may be substituted by one or more substituents independently selected from alkyl, haloalkyl, aminoalkyl, alkylhydroxyl, aryl, nitro, cyano, amino, alkylamino, arylamino, dialkylamino, mercapto halo, hydroxyl, alkoxy, alkylenedioxy, haloalkoxy, aryloxy, alkylcarbonylamino, alkylaminocarbonyl, alkylcarbonyl, aminocarbonyl, alkoxycarbonyl, arylcarbonyl, alkylthio or arylthio groups or may be substituted by a spiro, fused or spiro-fused cycloalkyl or heterocycloalkyl group which may be unsubstituted or substituted by alkyl, haloalkyl, aminoalkyl, arylalkyl, aryl, heteroaryl, nitro, cyano, amino, alkylamino, arylamino, dialkylamino, halo, hydroxyl, alkylhydroxyl, aryloxy, alkylcarbonylamino, alkylaminocarbonyl, alkylcarbonyl, aminocarbonyl, alkoxycarbonyl, arylcarbonyl or aryloxycarbonyl, wherein the aryl moieties of any of the above substituents are optionally substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups; or, when Q is CH, R⁸ may also be selected from halo, nitro, or mercapto;

and pharmaceutically acceptable salts, solvates, active metabolites, or prodrugs thereof.

In especially preferred embodiments, the compounds of this invention have the formula:

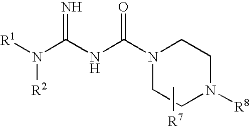

wherein R¹, R², and R⁷ are as defined above;

R⁷ is selected from H, substituted or unsubstituted lower alkyl, amino, alkylamino, dialkylamino, halo, keto (oxo), hydroxyalkyl, hydroxyl, alkoxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, wherein the alkyl or aryl moieties thereof may be substituted by one or more substituents independently selected from alkyl, haloalkyl, aminoalkyl, alkylhydroxyl, nitro, cyano, amino, alkylamino, dialkylamino, halo, hydroxyl, alkoxy, alkylenedioxy, haloalkoxy, alkylcarbonylamino, alkylaminocarbonyl, alkylcarbonyl, aminocarbonyl or alkoxycarbonyl groups;

R⁸ is selected from H, substituted or unsubstituted lower alkyl, cycloalkenyl, heteroaryl, heterocycloalkyl, arylalkyl, arylalkenyl, heteroarylalkyl, heterocycloalkylalkyl, aryl, cyano, amino, alkylamino, arylamino, dialkylamino, hydroxyalkyl, hydroxyl, alkoxy, haloalkoxy, aryloxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, arylcarbonyl, alkylthio or arylthio groups or may be substituted by a spiro, fused or spiro-fused cycloalkyl or heterocycloalkyl group which may be unsubstituted or substituted by alkyl, haloalkyl, aminoalkyl, arylalkyl, aryl, heteroaryl, nitro, cyano, amino, alkylamino, arylamino, dialkylamino, halo, hydroxyl, alkylhydroxyl, aryloxy, alkylcarbonylamino, alkylaminocarbonyl, alkylcarbonyl, aminocarbonyl, alkoxycarbonyl, arylcarbonyl or aryloxycarbonyl, wherein the alkyl, alkenyl, cycloalkyl, heterocycloalkyl, aryl or heteroaryl moieties thereof may be substituted by one or more substituents independently selected from alkyl, haloalkyl, aminoalkyl, alkylhydroxyl, aryl, nitro, cyano, amino, alkylamino, arylamino, dialkylamino, halo, hydroxyl, alkoxy, alkylenedioxy, haloalkoxy, aryloxy, alkylcarbonylamino, alkylaminocarbonyl, alkylcarbonyl, aminocarbonyl, alkoxycarbonyl, arylcarbonyl, mercapto, alkylthio or arylthio groups, wherein the aryl moieties of any of the above substituents are optionally substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups;

and pharmaceutically acceptable salts, solvates, active metabolites, or prodrugs thereof.

Exemplary compounds useful as 5-HT ligands according to this invention include the following:

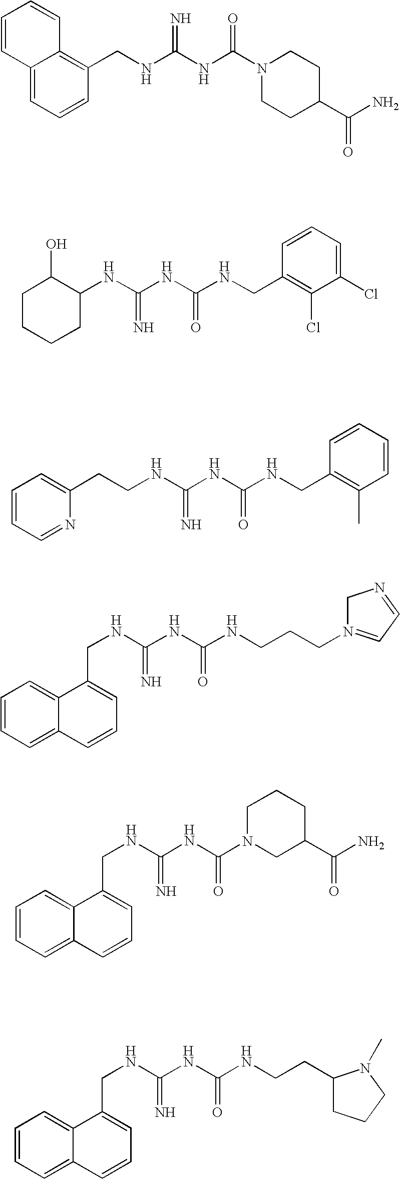

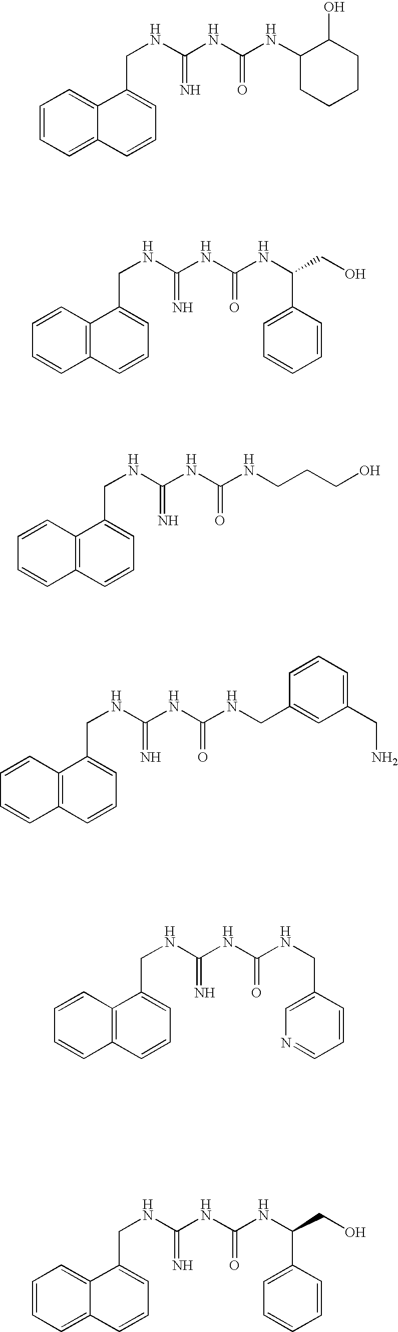

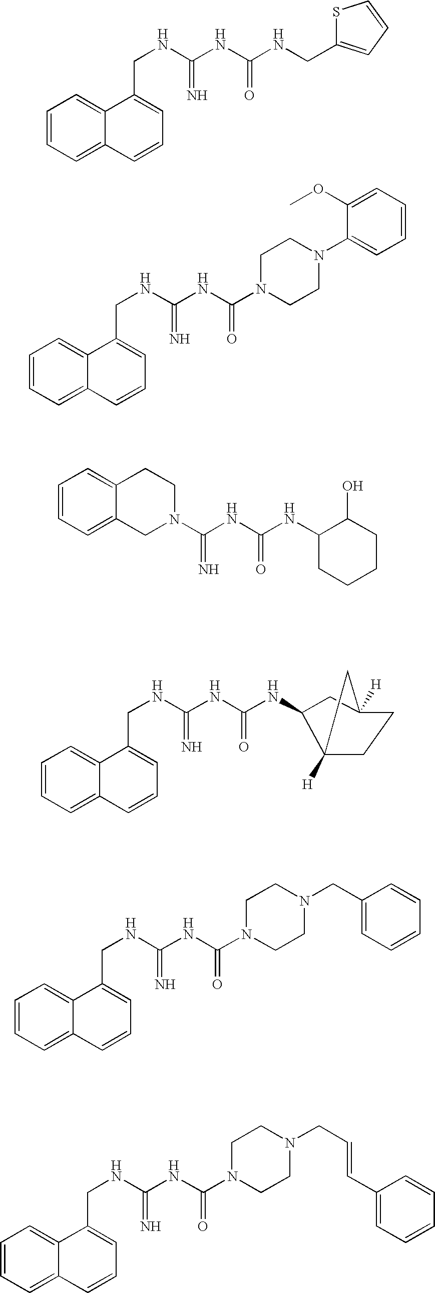

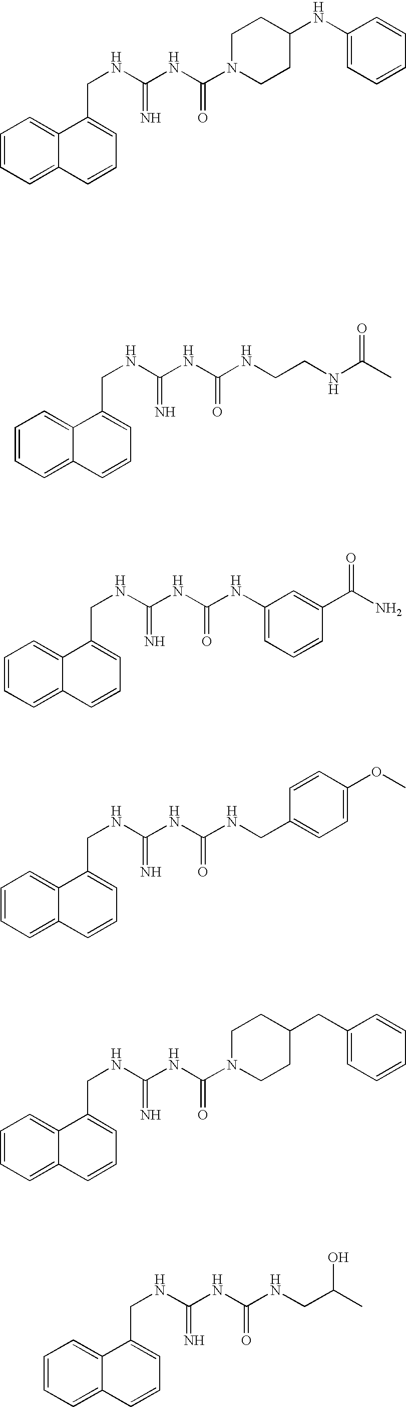

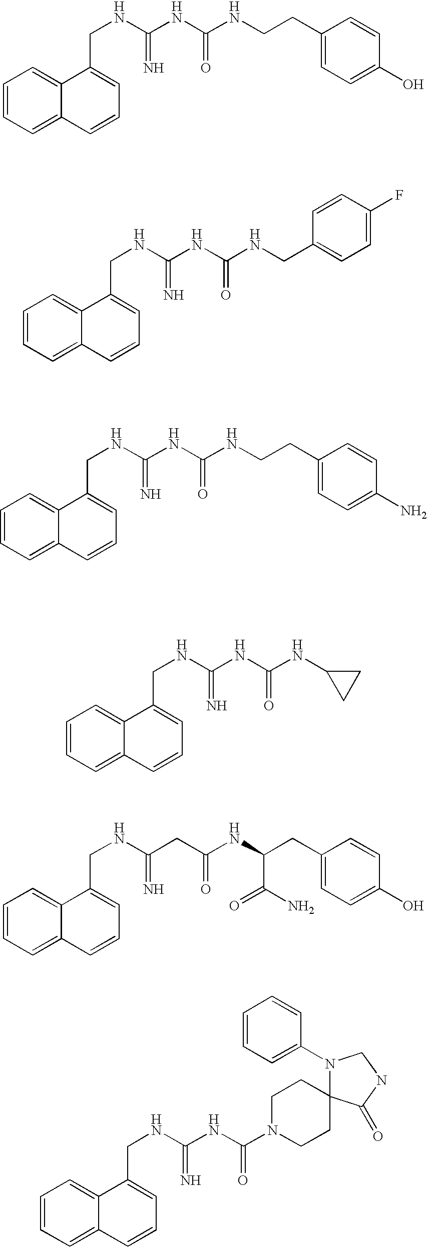

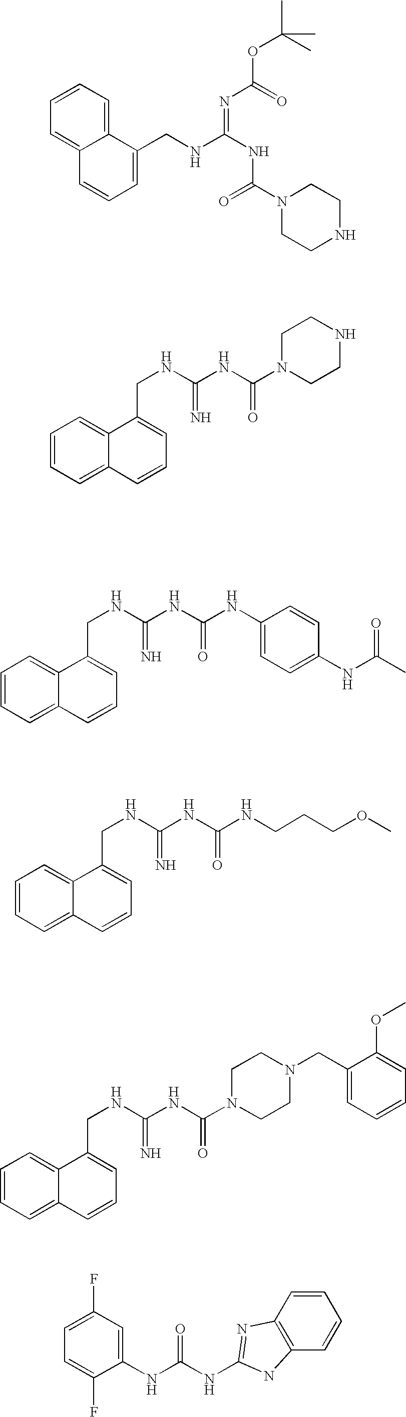

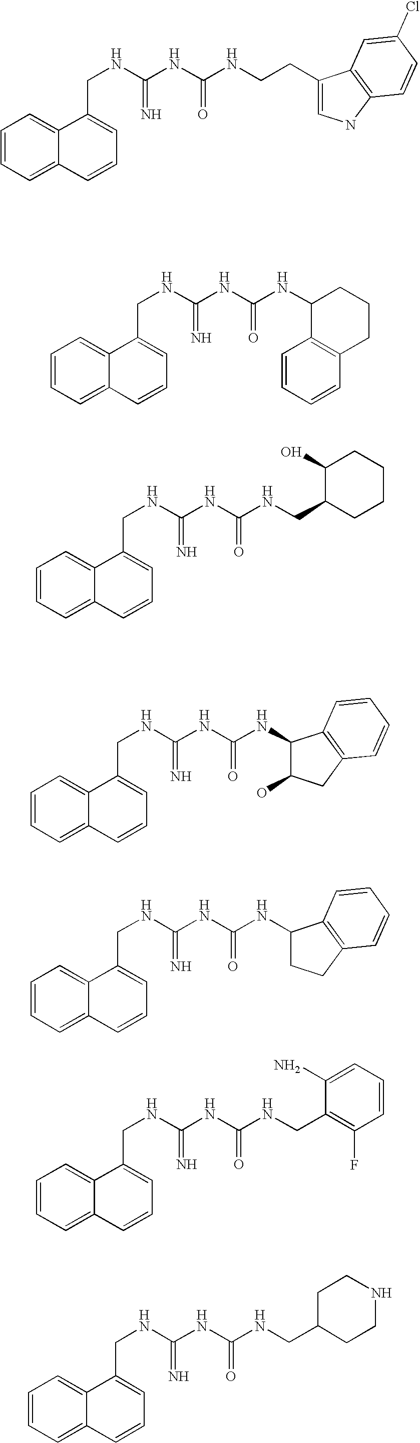

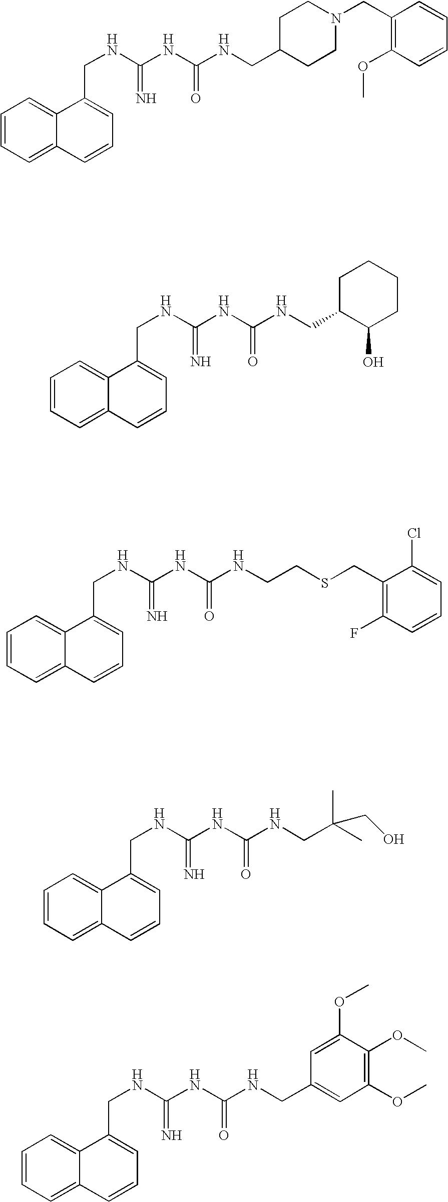

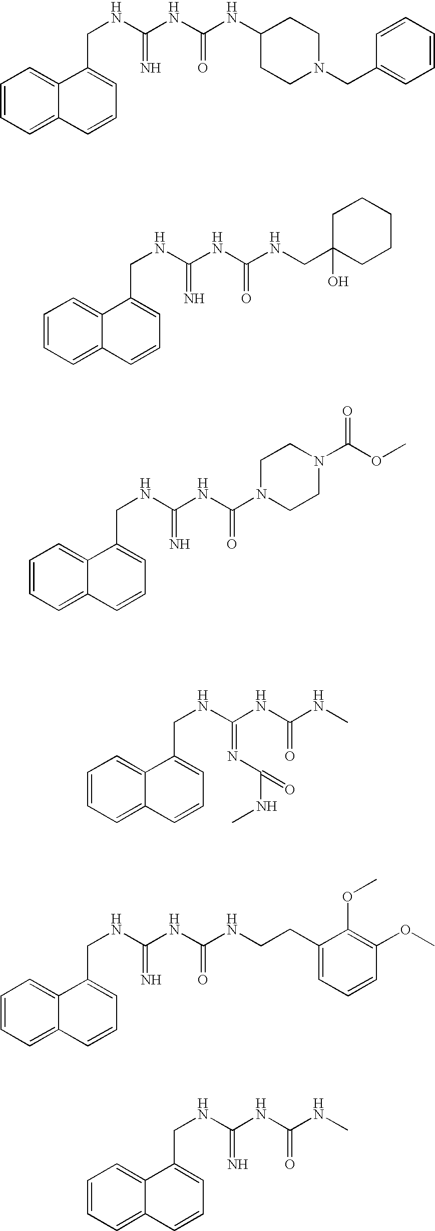

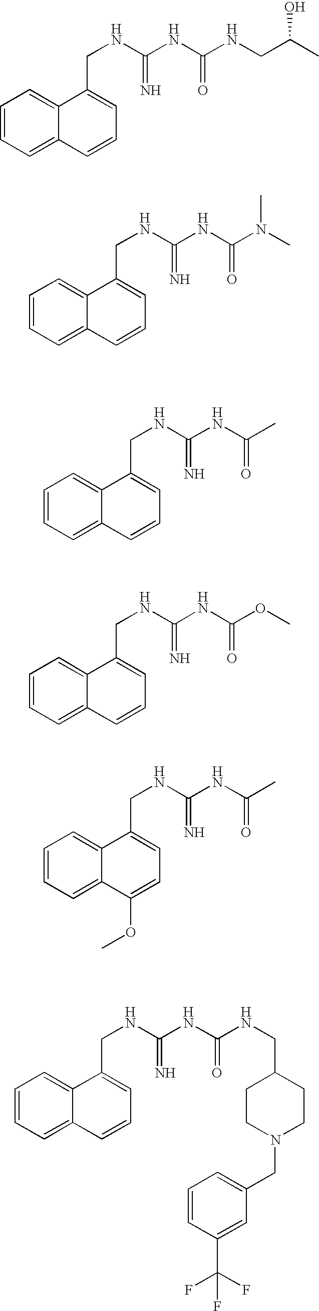

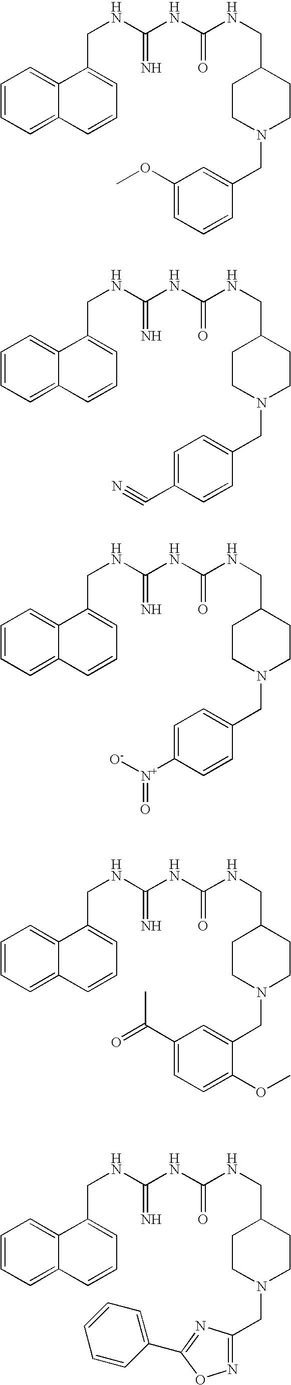

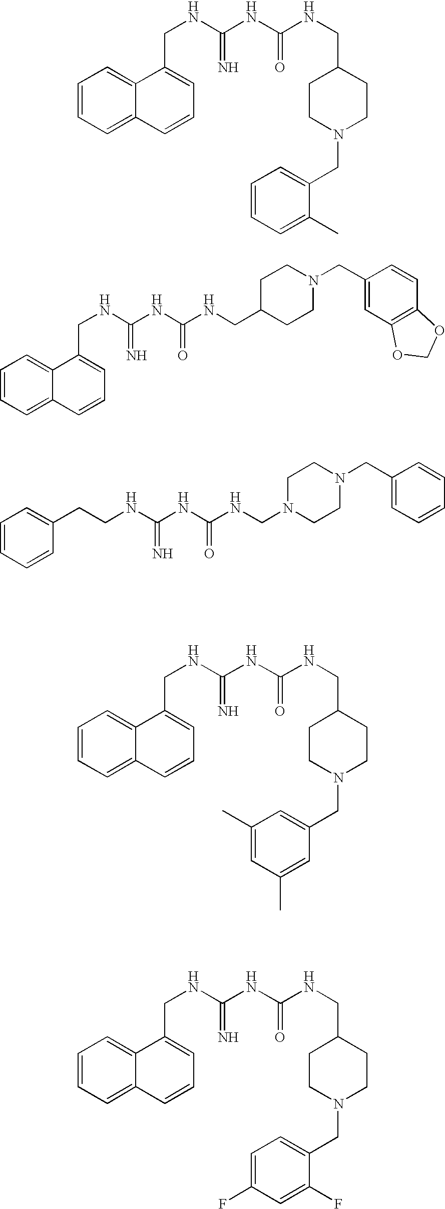

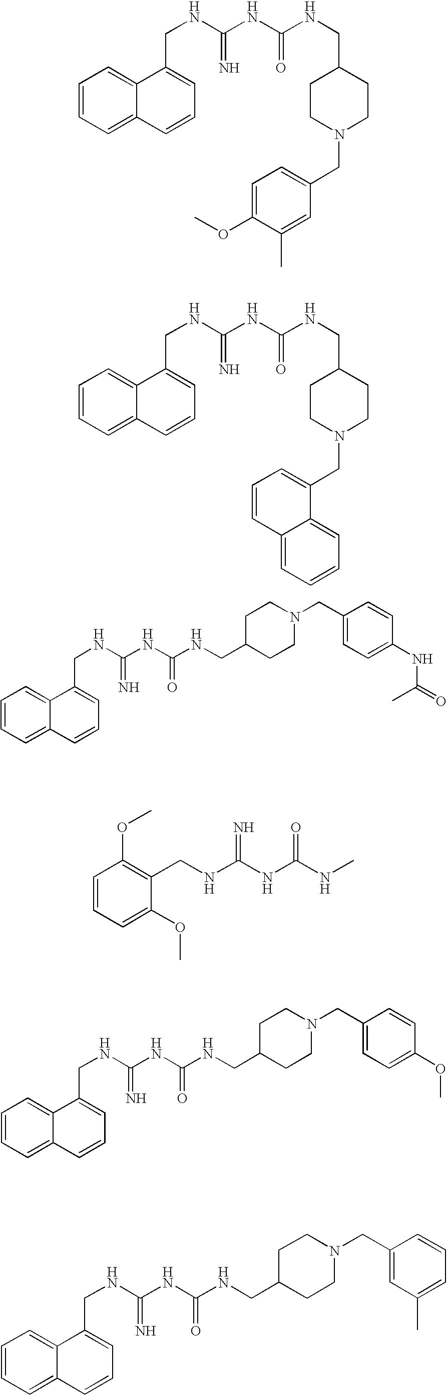

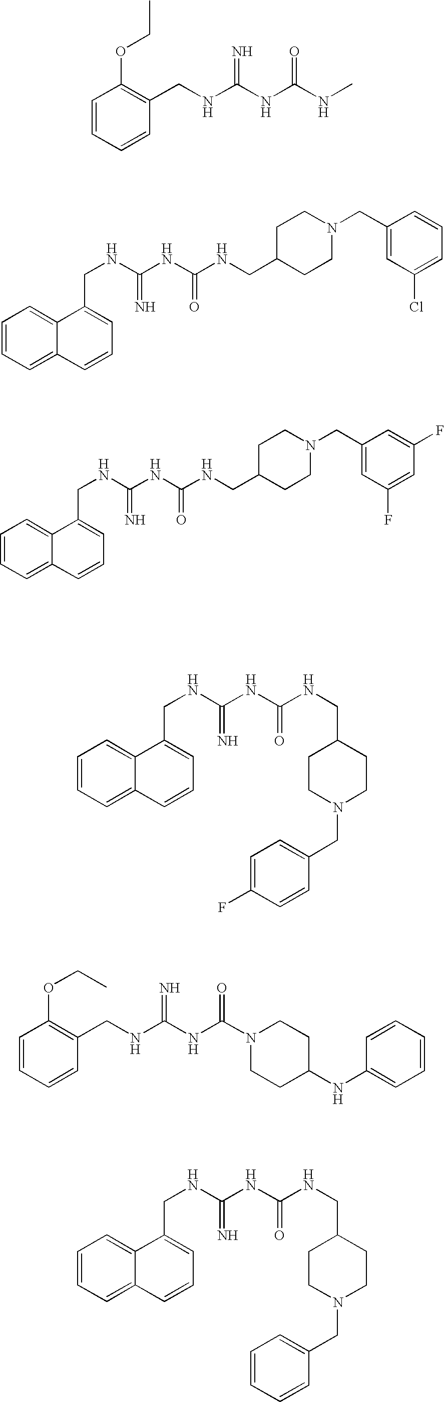

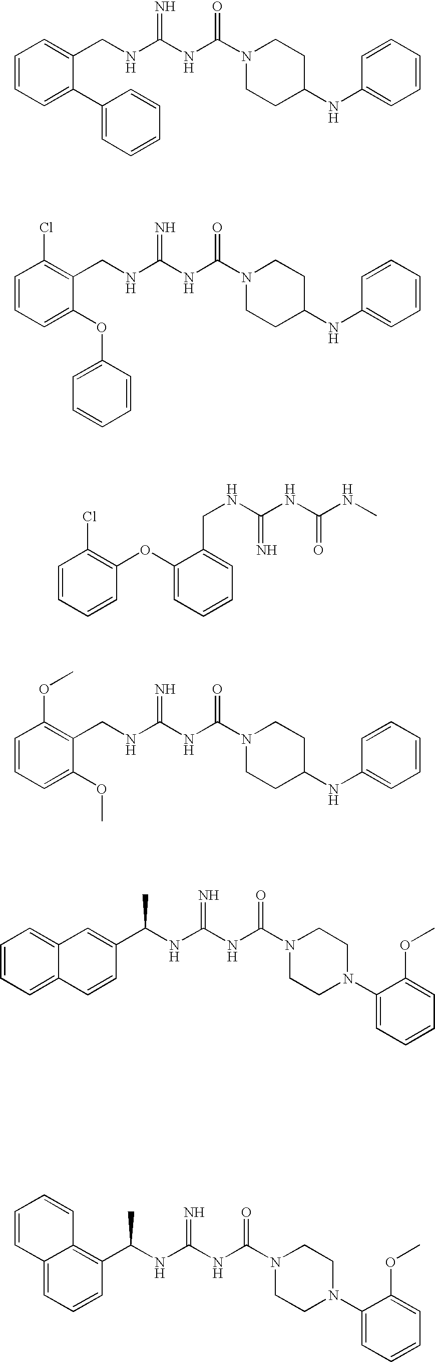

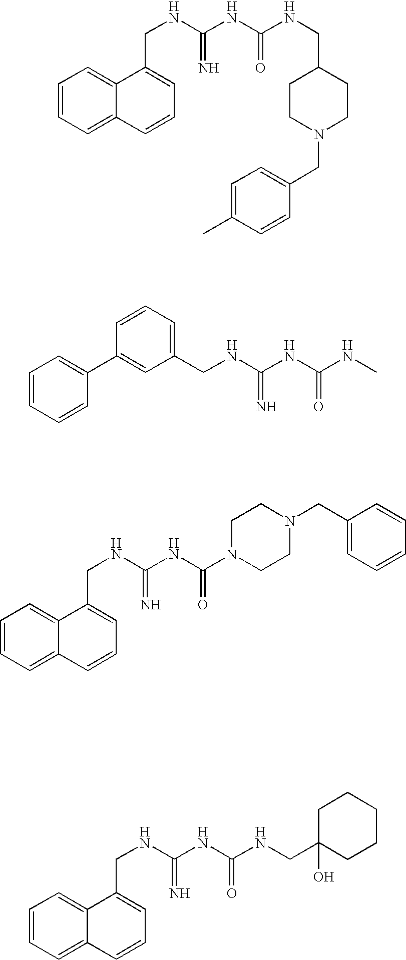

or pharmaceutically acceptable salts, solvates, active metabolites, or prodrugs thereof.

The invention also encompasses methods for preparing the compounds of Formula I and intermediates useful therein. Especially preferred intermediates used in the preparation of compounds of Formula I are the intermediate compounds of Formula II-A or II-B:

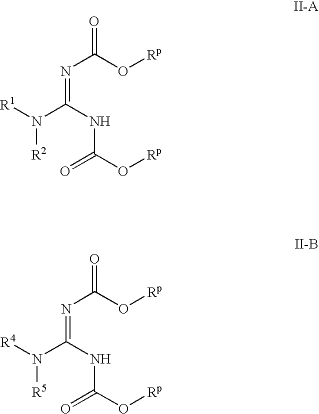

or are pharmaceutically acceptable salts thereof, wherein R¹, R², R⁴ and R⁵ are as defined above and, as above, R^{p} refers to the alkyl or aryl portion of a suitable nitrogen protecting group. In preferred examples, R^{p} is t-butyl, and the intermediate compound has the formula:

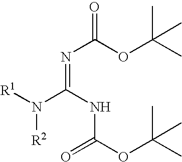

or pharmaceutically acceptable salts thereof. Exemplary intermediate compounds of this invention include, but are not limited to:

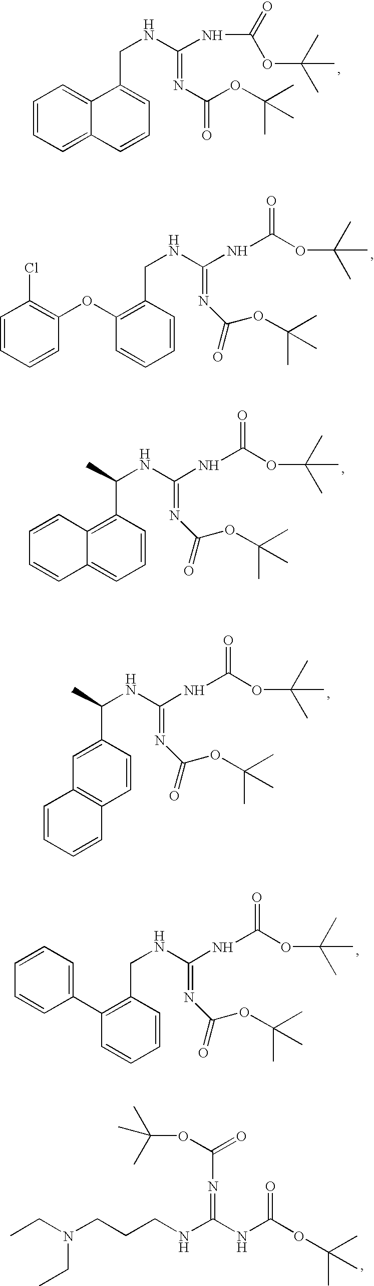

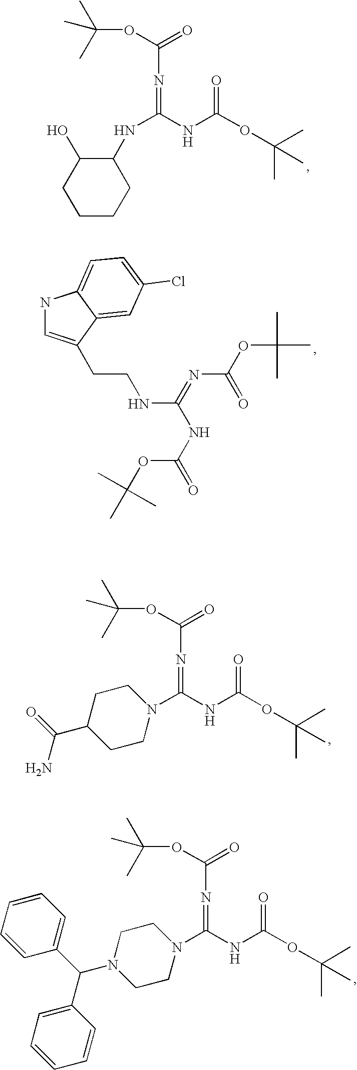

or pharmaceutically acceptable salts thereof

The compounds of the invention interact with 5-HT receptors and show selectivity for 5-HT receptors. The 5-HT receptor binding properties of the compounds are identified by competitive radioligand binding assays wherein membranes prepared from transfected cells expressing the 5-HT receptor subtype of interest. “Binding constants” refers herein to Kᵢ values measured by inhibition of the binding of radiolabelled ligands that are selective for the 5-HT receptor type being studied. For 5-HT₇ receptors, Kᵢ values are determined by measuring the inhibition of 5-carboxamidotryptamine (5-CT) binding, wherein 5-HT₇ receptors were incubated with the radiolabelled high affinity ligand, 5-carboxamidotryptamine ([³H]5-CT), in the presence and absence of the compounds of the invention, at varying concentrations. The compounds of the invention have high binding affinity for serotonin receptors as measured by dissociation constant Kᵢ. The compounds of the present invention preferably show 5-HT₇ receptor binding characterized by Kᵢ values less than about 100 nM, more preferably by Kᵢ values less than about 10 nM, and most preferably by Kᵢ values less than about 1 nM. “Selectivity” for receptor type, in the context of this invention, refers to the ratio of binding constants for the two receptor types being compared. For example, if a hypothetical ligand shows Kᵢ of 100 nM for 5-HT₄ receptors and 0.5 nM for 5-HT₇ receptors, its selectivity for 5-HT₇ over 5-HT₄ receptors is 200-fold. The compounds of the present invention preferably show selectivity for 5-HT₇ receptors over other serotonin receptor subtypes of greater than about 100. The compounds of the present invention also preferably show selectivity for 5-HT₇ receptors over other receptor types, such as dopamine D2, of greater than about 100.

The compounds of the invention interact with 5-HT receptors and act as antagonists at that receptor. The agonist or antagonist properties of the compounds were measured by the ability of the compounds to increase basal or to inhibit 5-HT-stimulated c-AMP formation in transfected cells expressing 5-HT₇ receptors. The biological activity of the inventive compounds is determined by assays that have been devised to serve as animal models for various human medical conditions. Many such assays are known to skilled practitioners. Examples of such assays include, for example:

the prokinetic assay, which is an in vivo method of determining the extent the test compound affects the rate of gastric emptying of a test meal in rats;

the anxiolytic behavior assay, which measures the extent to which the test compound can ameliorate of the symptoms of natural anxiety in mice when exposed to a novel, brightly lighted environment;

the withdrawal anxiety assay, which measures the extent to which the test compound can ameliorate of the symptoms in mice caused by withdrawal from addictive substances by measuring the extent the drug affects the anxiety that occurs in mice after chronically treating with an addictive substance and then abruptly ceasing the treatments;

the cognitive enhancement assay, which measures the extent the test compound can alleviate the cognitive deficit induced in rats by administration of atropine to rats.

These assays are described in U.S. Pat. No. 5,763,468, the disclosure of which s hereby incorporated herein by reference.

The invention encompasses pharmaceutical compositions comprising compounds of Formula I, or a pharmaceutically acceptable salt, solvate, active metabolite, or prodrug thereof, and treatment of a patient in need thereof with a pharmaceutical composition comprising an effective amount of a Formula I compound, or a pharmaceutically acceptable salt, solvate, active metabolite, or prodrug thereof. As 5-HT₇ receptor ligands, the compounds of the invention are useful for treating conditions which can be ameliorated by interaction with 5-HT₇ receptors. Such conditions include sleep disorders, depression, pain, and schizophrenia.

A Aprodrug” is intended to mean a compound that is converted under physiological conditions or by solvolysis or metabolically to a specified compound that is pharmaceutically active. A “pharmaceutically active metabolite” is intended to mean a pharmacologically active compound produced through metabolism in the body of a specified compound. Prodrugs and active metabolites of compounds of Formulas I-V may be determined using techniques known in the art, for example, through metabolic studies. See, e.g., ADesign of Prodrugs,@ (Bundgaard, ed.), 1985, Elsevier Publishers B. V., Amsterdam, The Netherlands.

A “pharmaceutically acceptable salt” is intended to mean a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates. A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine. In the case of compounds, salts, or solvates that are solids, it is understood by those skilled in the art that the inventive compounds, salts, and solvates may exist in different crystal forms, all of which are intended to be within the scope of the present invention and specified formulas.

Administration of the compounds of the invention and their pharmaceutically acceptable prodrugs, salts, active metabolites, and solvates may be performed according to any of the accepted modes of administration available to those skilled in the art. Illustrative examples of suitable modes of administration include oral, systemic (e.g., transdermal, intranasal, or by suppository), parenteral (e.g., intramuscular, intravenous, or subcutaneous), topical, transdermal and rectal. An inventive compound or a pharmaceutically acceptable salt, prodrug, active metabolite, or solvate thereof may be administered as a pharmaceutical composition in any pharmaceutical form recognizable to the skilled artisan as being suitable. Suitable pharmaceutical forms include solid, semisolid, liquid, or lyophilized formulations, such as tablets, powders, capsules, suppositories, suspensions, liposomes, and aerosols. Pharmaceutical compositions of the invention may also include suitable excipients, diluents, vehicles, and carriers, as well as other pharmaceutically active agents, depending upon the intended use or mode of administration. Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixr, emulsion, soft gelatin capsule, sterile injectable liquid (e.g., solution), or a nonaqueous or aqueous liquid suspension.

The compounds (active ingredients) may be formulated into solid oral dosage forms which may contain, but are not limited to, the following inactive ingredients: diluents (i.e., lactose, corn starch, microcrystalline cellulose), binders (i.e., povidone, hydroxypropyl methylcellulose), disintegrants (i.e., crospovidone, croscarmellose sodium), lubricants (i.e., magnesium stearate, stearic acid), and colorants (FD&C lakes or dyes). Alternatively, the compounds may be formulated into other oral dosage forms including liquids, suspensions, emulsions, or soft gelatin capsules, with each dosage form having a unique set of ingredients.

A dose of the pharmaceutical composition contains at least a therapeutically effective amount of the active compound or agent (i.e., an inventive compound or a pharmaceutically acceptable salt, prodrug, active metabolite, or solvate thereof), and preferably is made up of one or more pharmaceutical dosage units. The selected dose may be administered to a mammal, for example, a human patient, in need of treatment mediated by inhibition of serotonin agonist activity, by any known or suitable method of administering the dose, including topically, for example, as an ointment or cream; orally; rectally, for example, as a suppository; parenterally by injection; or continuously by intravaginal, intranasal, intrabronchial, intraaural, or intraocular infusion. A “therapeutically effective amount” is intended to mean the amount of an inventive compound that, when administered to a mammal in need thereof, is sufficient to effect treatment for disease conditions alleviated by the inhibition of the action of serotonin at the 5-HT receptor. The amount of a given compound of the invention that will be therapeutically effective will vary depending upon factors such as the particular compound, the disease condition and the severity thereof, the age and health of the subject in need of treatment, which may be routinely determined by skilled artisans.

The Examples that follow are intended as illustrations of certain preferred embodiments of the invention, and no limitation of the invention is implied. It is considered within the skill of one in the art to recognize that the chemical reactions described herein are generally applicable to prepare other compounds encompassed within the scope of the invention, or that such compounds may be prepared by appropriate modification of these illustrated reactions or use of analogous or other conventional synthetic methods known in the art, without undue experimentation (e.g., by use of appropriate blocking or protecting groups, by substituting other conventional reagents, or by routine modifications of reaction conditions). Although certain protecting groups are exemplified in the syntheses described below, it is understood that other suitable protecting groups may be used, depending on the finctionality present in the desired compound and intermediates required for the preparation thereof, and depending on the particular synthesis method employed

In each of the synthetic procedures described herein, unless otherwise indicated, the starting materials are known, available, or may be readily prepared from known starting materials, all temperatures are set forth in degrees Celsius, and all parts and percentages are by weight. Reagents were purchased from commercial suppliers, such as Aldrich Chemical Company or Lancaster Synthesis Ltd. Reagents and solvents were commercial grades and were used as supplied. ¹H-NMR (300 MHz) spectra were measured in CDCl₃ solutions unless otherwise indicated and were determined on a Bruker DRX-300 instrument using XWIN NMR Version 1.2 operating software. Chemical shifts are reported in parts per million (ppm) downfield from tetramethylsilane as the internal standard, and coupling constants are given in Hertz. The following abbreviations are used for spin multiplicity: br=broad, s=singlet, d=doublet, t=triplet, q=quartet, m=multiplet, and cm=complex multiplex. Infrared (IR) spectra were recorded on a Perkin-Elmer 1600 series FTIR spectrometer and are reported in wavenumbers (cm⁻¹). Elemental analyses were performed by Atlantic Microlab, Inc., Norcross, Ga. High-resolution mass spectra (HRMS) were performed by Scripps Mass Spectra Laboratory, La Jolla, Calif. Melting points (mp) were determined on a Mel-Temp II apparatus and are uncorrected. Unless otherwise indicated, the reactions set forth below were carried out under a positive pressure with a balloon of nitrogen (N₂) or argon (Ar) at ambient temperature in anhydrous solvents, and the reaction flasks were fitted with rubber septa for the introduction of substrates and reagents via syringe. Glassware was heat-dried. Analytical thin-layer chromatography (TLC) was performed on glass-backed silica gel 60 F 254 plates (Analtech, 0.25 mm) and eluted with the appropriate solvent ratios (v/v), which are denoted where appropriate. The reactions were assayed by TLC and terminated as judged by the consumption of starting material. The tip plates were visualized using an ultraviolet (UV) lamp. Visualization can also be accomplished using stains such as potassium permanganate, ninhydrin, ammonium molybdate, iodine (I₂) chamber, or p-anisaldehyde spray reagent or phosphomolybdic acid reagent (Aldrich Chemical, 20 wt % in ethanol) activated with heat.

Recovery of the desired compounds from the reaction mixtures described herein was typically accomplished by doubling the reaction volume with the reaction solvent or extraction solvent and washing with the indicated aqueous solutions using 25% by volume of the extraction volume (unless otherwise indicated). Product solutions were dried over anhydrous Na₂SO₄ prior to filtration and evaporation of the solvents was conducted under reduced pressure on a rotary evaporator. Purification of products and intermediates was conducted by flash column chromatography using silica gel 60 (Merck Art 9385). (Still et al., J. Org. Chem. 43:2923 (1978)) was done using silica gel 60 (Merck Art 9385):crude material ratio of about 20:1 to 50:1 (unless otherwise indicated). Hydrogenolyses were performed at the pressures indicated in the examples or at ambient pressure.

The Formula I compounds of the invention may be prepared by straighitforward modifications to the general method depicted below. Modifications include variations in starting materials, as will be obvious to artisans. The method of this invention comprises treatment of a 1-H-pyrazole-1-(N-(nitrogen-protected))carboxamidine I-1 with a carbonylating agent (e.g., an anhydride, a carboxylic acid halide (acid chloride) or a haloformate (chloroformate)) to form the di-carboxylated intermediate I-2. Preparation of the compounds of formula I-B may be accomplished, for example, by treating I-1 with an anhydride or an acid halide as the carbonylating agent (e.g., (RCO)₂O or RCOCl, respectively, wherein R=—CHR⁴R⁵) wherein the di-carboxylated intermediate I-2 is a carbamate-amide. Treatment of I-2 with HNR¹R² forms the amino-di-carboxylated intermediate I-3. Removal of the nitrogen protecting group (—C(O)OR^{P}) from I-3 provides compounds of formula I-B.

Alternatively, the carbonylating agent may be a precursor for a suitable nitrogen protecting group, C(O)OR^{P}; e.g., the carbonylating agent may be di-tert-butyl dicarbonate, 2-(tert-butoxycarbonyloxyimino)-2-phenylacetonitrile or may be a formylating agent, such as benzyl chloroformate). Treatment of I-1 with such a carbonylating agent forms di-carboxylated intermediate I-2 as a di-carbamate. In the embodiment of this general method shown below, R=OR^{p}, wherein R^{p} refers to a moiety of the nitrogen protecting group. For example, when the protecting group is Boc, R^{p} is t-butyl (R is t-butyloxy) or when the protecting group is Cbz, R^{p} is benzyl (R is benzyloxy). Preferably, the protecting group is Boc and R^{p} is t-butyl (R is t-butyloxy). Treatment of I-2 with HNR¹R² forms the amino-di-carbamate intermediate I-3, which may then be treated with a precursor reagent of the ZR⁴R⁵ group (HNR⁴R⁵ to form the precursor compounds of formula I-A or R⁴OH to form the precursor compounds of formula I-C). Removal of the nitrogen protecting groups (C(O)OR^{p}) provides the compounds of Formulas I-A and I-C.

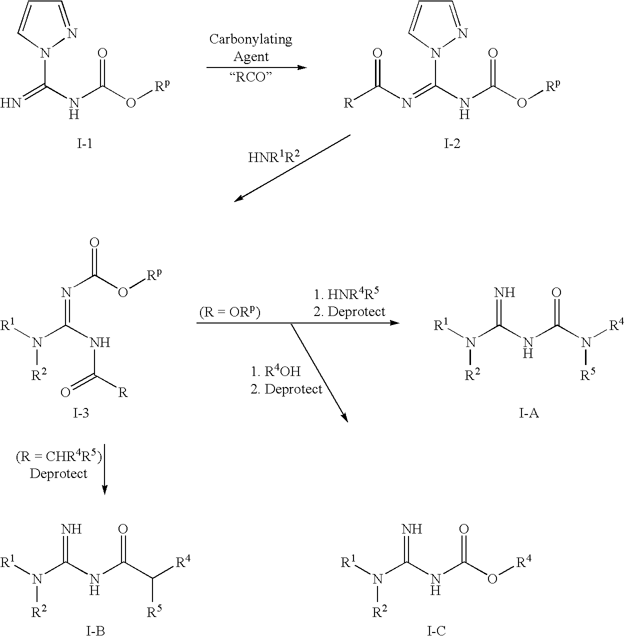

In the following descriptions, the exemplary nitrogen protecting group R^{P} is Boc (t-butyloxycarbonyl), but other alternative suitable protecting groups for nitrogen may be employed.

General Synthetic Method I as described below may be used to prepare amidinourea compounds of formula D.

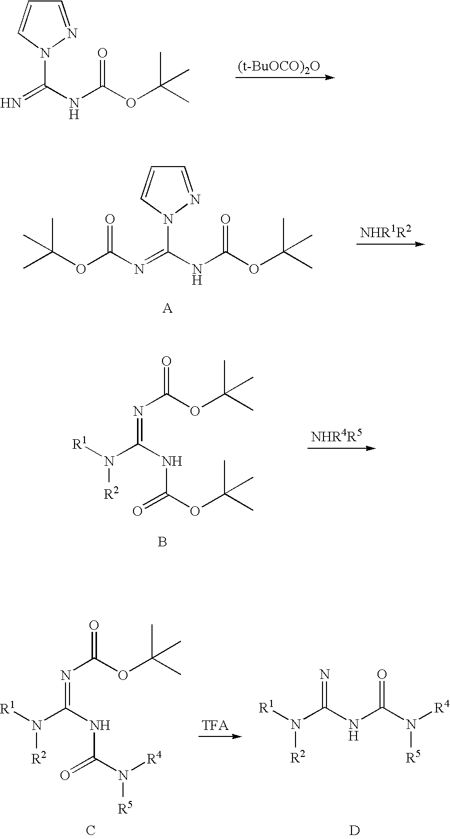

Preparation of N, N′-diBoc-guanidines (B): To a solution of the amine NHR¹R² in THF (0.2 M) is added as a solid 1-H-pyrazole-1(N,N′-bis(tert-butoxycarbonyl)carboxamidine (1.0 equiv.) at room temperature. The solution is stirred at room temperature for 2 hours. The solvent is removed under reduced pressure. The residue obtained is dissolved in 2 times the volume amount of THF used in the reaction and washed with water. The oil layer is separated, dried over MgSO₄ and concentrated. The product is purified by silica gel column chromatography eluted with hexane/ethyl acetate (9:1). The solvent is removed under reduced pressure to afford product B. The typical TLC conditions are 5:1 hexane/ethyl acetate and typical yields range from 70% to 90%.

Preparation of N-Boc-guanylureas (C): A solution of N,N′-diBoc-guanidine B (1.0 equiv.) prepared as above and the amine NHR⁵R⁶ (1.0 equiv.) in THF (0.3 M) is heated to reflux for 8 hours. After the solution is cooled to room temperature the solvent is removed under reduced pressure. The residue is purified by silica gel column chromatography eluted with hexane/ethyl acetate (18:1 to 1:1) to afford the mono-Boc-guanylurea compound C. The typical TLC conditions are 5:1 to 1:1 hexane/ethyl acetate. The yield of the reaction is normally between 60-90%. The procedure is described by Gregor, V. E.; Hong, Y.; Ling, A. L.; Tompkins, E. V “Neuropeptide-Y-Ligands” International Publication No. WO 98/07420; and Miel, H.; Rault, S.; Tetrahedron Letters. 1998, 39, 1565-1568.

Preparation of amidino-urea compounds (D): The monoBoc-protected amidinourea product C is dissolved in a solution of 50% TFA in dichloromethane (0.1 M). The reaction contents are stirred at room temperature for 30-60 minutes. The reaction solvent and excess amount of TFA are removed under reduced pressure. The residue is dissolved in dichloromethane, poured into water and basicified with 5% NaOH to pH 9˜11. The separated organic layer is dried over MgSO₄ and concentrated. The crude product is purified by silica gel chromatography eluted with a mixture of CH₂Cl₂MeOH (1˜15% MeOH) to give the amidinourea D. The typical yields range from 85-100%. These compounds can also be purified by high-performance liquid chromatography (HPLC) using a water/acetonitrile/TFA solvent system. The starting material 1-H-pyrazole-1-(N,N-bis(tert-butoxycarbonyl)carboxamidine is prepared according to Drake et al. (Synth., 1994, 579-582).

Alternative procedure for preparation of amidino-urea compounds (D): The guanyllating reagent A (1.0 equiv.), the amine NR¹R² (1.0 equiv.), and THF (0.3 M) are placed into a reaction vessel. The reaction mixture is stirred at room temperature for 2 hours. To this solution is added amine NR⁴R⁵ (1.0 equiv.), and the solution is heated to reflux overnight. The solvent is removed under reduced pressure. The residue is purified by silica gel column chromatography to afford C. Typical TLC conditions are 5:1 to 1:1 hexane/ethyl acetate. Compound C is then treated with 50% TFA/CH₂Cl₂ and purified with silica gel column chromatography or HPLC to give D. When this procedure is applied to production of combinatorial chemistry compounds, C is usually not isolated.

General Synthetic Method II is used to prepare compounds of formula G.

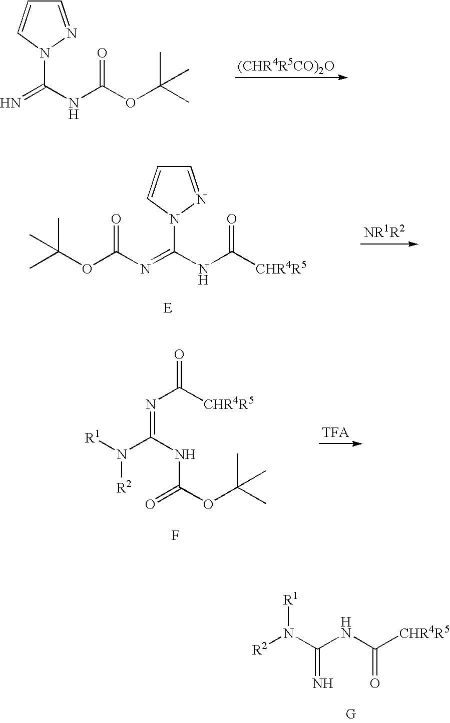

Acylation: To a solution of 1-H-pyrazole-1-(N-tert-butoxycarbonyl) carboxamidine in THF (0.5 M) is added diisopropylethylamine (1.5 equiv.) and anhydride (CHR⁴R⁵CO)₂O (1.5 equiv.). The solution is heated to reflux for 12 hours under N₂ atmosphere. The reaction mixture is extracted with ethyl acetate. The separated organic layers are washed with aqueous brine, dried over MgSO₄ and concentrated on a rotary evaporator. The residue is purified by silica gel chromatography eluted with hexane/ethyl acetate (6:1). The yield ranges between 30% and 50%.

Preparation of N-Boc-acylguanidines: A solution of 1-H-pyrazole-1-(N-tertbutoxycarbonyl-N′-acyl)carboxamidine E prepared in step 1 and the amine NHR¹R² (1.0 equiv.) in THF (0.3 M) is stirred at room temperature for 8 hours. The solvent is removed under reduced pressure. The residue is purified by silica gel chromatography eluted with hexane/ethyl acetate (5:1 to 2:1) to afford N-Boc-acylguanidine F. The typical TLC conditions are 5:1 to 1:1 hexane/ethyl acetate. The yield of the reaction is normally between 60% and 80%.

Preparation of acylguanidine compounds: The N-Boc-protected acylguanidine product E is dissolved in a solution of 50% TFA in dichloromethane (0.1 M). The reaction contents are stirred at room temperature for 30-60 minutes. The reaction solvent and excess amount of TFA are removed under reduced pressure. The residue is dissolved in dichloromethane, poured into water and basicified with 5% NaOH to pH 9 to 11. The separated organic layer is dried over MgSO₄ and concentrated. The crude product is purified on a silica gel column eluted with CH₂Cl₂/MeOH (1% to 5% MeOH) to give the acylguanidine G. The typical yields range from 85% to 100%. The compounds of formula G may also be purified by high-performance liquid chromatography (HPLC) using a water/acetonitrile/TFA solvent system.

General Synthetic Method III may be used to prepare compounds of formula J.

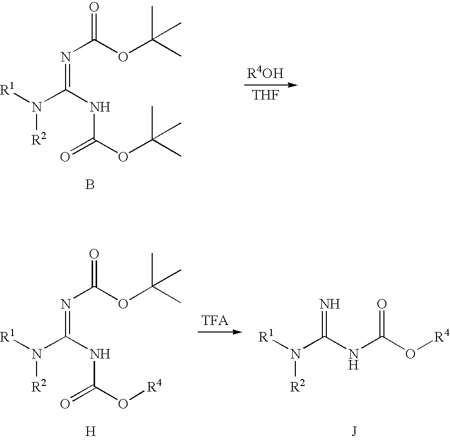

Preparation of N-Boc-guanidinylesters (H): A solution of N,N′-di-Boc-guanidine (1.0 equiv.) and alcohol R⁴OH (5.0 equiv.) in THF (0.3 M) is heated to reflux for 8 hours. The solvent is removed under reduced pressure. The residue is purified by chromatography on a silica gel column eluted with hexane/ethyl acetate (4:1) to give N-Boc-guanylester H. The typical TLC conditions are 3:1 hexane/ethyl acetate. The yield of the reaction is normally between 80% and 100%.

Preparation of guanidinylesters (J): The N-Boc-guanidinylester product H obtained as above is dissolved in a solution of 50% TFA in dichloromethane (0.1 M). The reaction contents are stirred at room temperature for 30-60 minutes. The reaction solvent and excess TFA are removed under reduced pressure. The residue is dissolved in dichloromethane, poured into water and basicified with 5% NaOH to pH 9 to 11. The separated organic layer is dried over MgSO₄ and concentrated. The crude product is purified on a silica gel column eluted with CH₂Cl₂/MeOH (1% to 5% MeOH) to give the guanidinylester J. The typical yields range from 85% to 100%. Compounds of formula J can also be purified by high-performance liquid chromatography (HPLC) using a water/acetonitrile/TFA solvent system.

Compounds having the formula wherein R⁴ and R⁵ together with Z form a 5- or 6-membered ring:

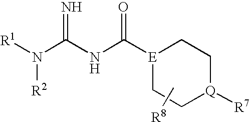

may be prepared using the general approaches described in General Method IV and General Method V, below.

General Synthetic Method IV

The general approach for preparation of compounds wherein R⁴ and R⁵ together with Z form a 5- or 6-membered ring is as shown below, where a further alkylation step is accomplished by treatment with an alkyl halide R⁸-halide:

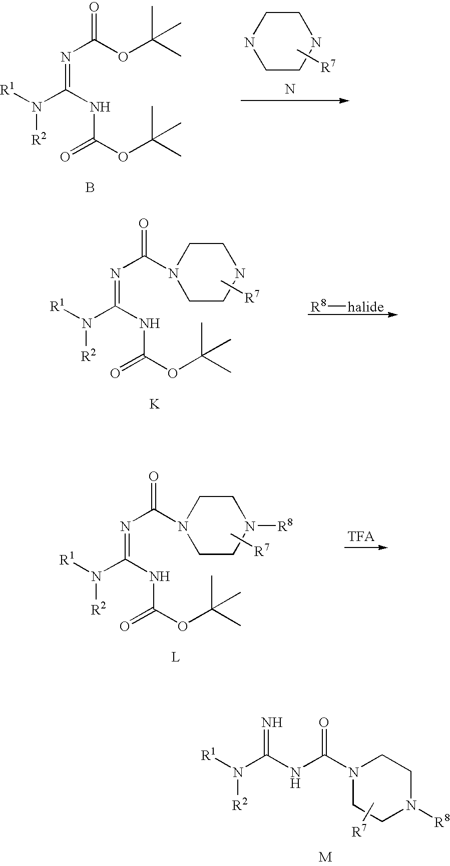

Preparation of (K): To a solution of a heterocyclic amine compound N (1.0 to 2.0 equiv.) in THF (0.2 M) is added as a solid 1-H-pyrazole-1(N,N′-bis(tert-butoxycarbonyl)carboxamidine B (1.0 equiv.) at room temperature. The solution is stirred at room temperature for 2 hours. The solvent is removed under reduced pressure. The residue obtained is dissolved in 2 times the volume amount of THF used in the reaction and washed with water. The oil layers are separated, washed with water and brine, dried over MgSO₄ and concentrated. The product is purified by column chromatography on silica gel eluted with methylene chloride/methanol (95:5). The solvent is removed under reduced pressure to afford the desired product K. The typical TLC conditions are 5% methanol in dichloromethane and typical yields range from 70% to 95%.

N-alkylation by alkylhalides, preparation of (L): The compounds of general structure L may be prepared by N-alkylation of the terminal amino group of K. The R⁸ group may be introduced by reaction with an appropriate alkyl halide R⁸-halide under basic conditions. To a solution of K (1 equiv.) in DMF or DMSO (0.2 M) is added K₂CO₃ (5 equiv.) and the alkyl halide R⁸-halide (1.0 equiv.). The reaction mixture is stirred at room temperature, or at elevated reaction temperatures depending on the reactivity of the alkyl halide, for 2 hours. The mixture is extracted with ethyl acetate twice. The separated organic layers are washed with water/brine, dried over MgSO₄ and concentrated on a rotary evaporator. The residue is purified by silica gel chromatography eluted with CH₂Cl₂/MeOH (1 to 15%) to give compound L.

The alkylation procedure may also be applied to related compounds O with a free guanidinyl group:

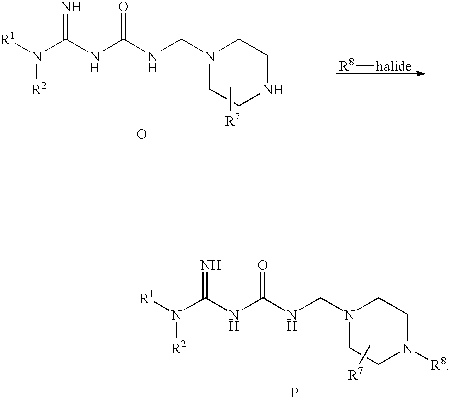

Alternatively, if the alkyl halides are highly activated such as benzyl halides or allyl halides, the alkylation reaction may be performed under the following conditions. The guanylurea compound K (1.0 equiv.) and alkyl halide R⁸-halide (1.0 equiv.) are dissolved in dichloromethane (0.2 M). To this solution is added triethylamine (2.0 equiv.). The solution is stirred at room temperature for 12 hours. The reaction mixture is extracted with CH₂Cl₂. The organic layers are concentrated on a rotary evaporator. The product L is purified by silica gel column chromatography eluting with methylene chloride/methanol.

Examples of alkyl halides useful in the above alkylation procedures include:

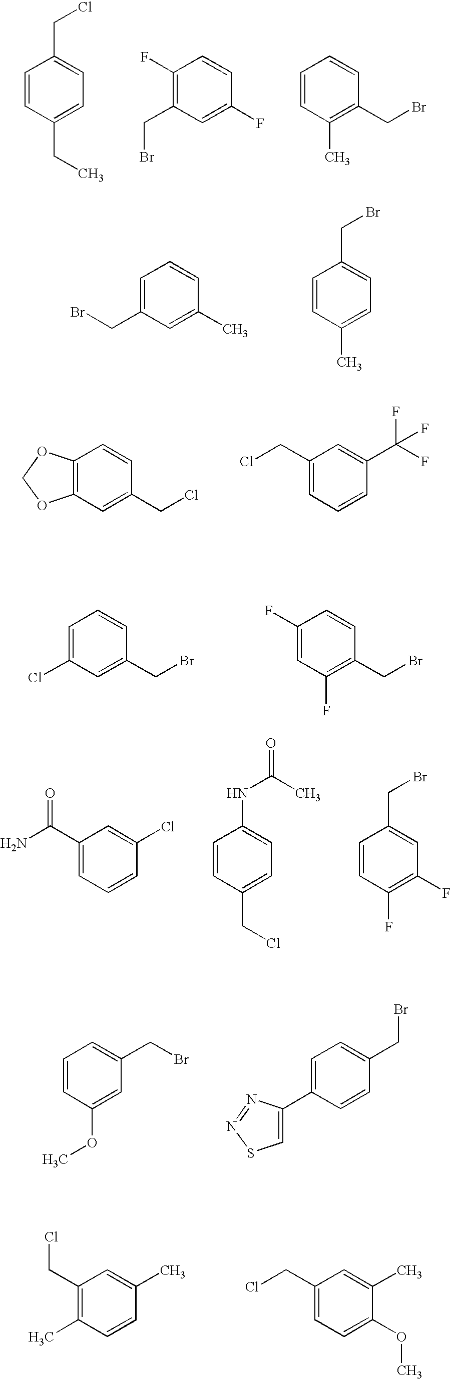

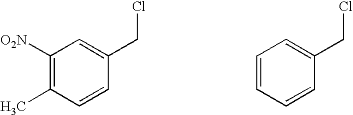

Deprotection: Deprotection of compound L maybe carried out according to the general procedure described in General Synthetic Method I for the synthesis of compound D.

General Synthetic Method V

Alternativley, the N-alkylated compounds P may be prepared by a method whereby the alkylation step is accomplished by reductive amination of an aldehyde R⁹—CHO, wherein R⁹ is selected such that —CH₂R⁹=R⁸ in the product compound R.

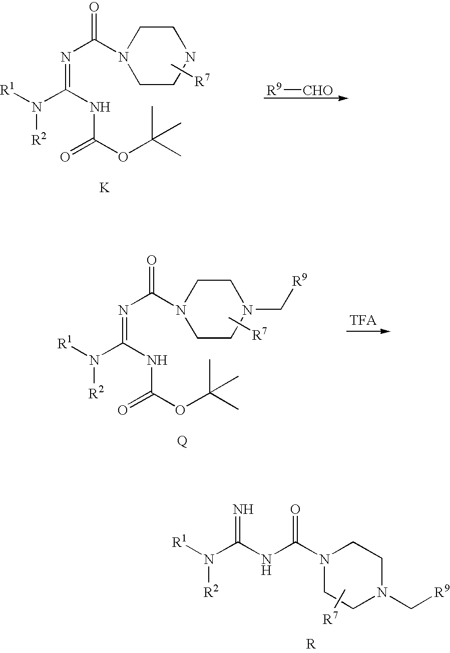

Reductive amination: To a solution of amine K and aldehyde R⁹CHO in acetonitrile (0.4M) is added sodium triacetoxyborohydride (2.5 equiv.). The solution is stirred at room temperature under nitrogen for 6 hours. To this solution is added a solution of saturated Na₂CO₃. The solution is stirred for 20 minutes. The reaction mixture is extracted by ethyl acetate two times in separator funnel. The organic layers are washed with water and brine, dried over MgSO₄ and concentrated under reduced pressure. The residue is purified by column chromatography to give compound Q. Reductive amination of aldehydes and ketones with cyclic amines such as piperazine and piperidine are described by Abdel-Magid, A. F.; Carson, K. G.; Harris, B. D.; Maryanoff, C. A.; Shah, R. D. J. Org. Chem. 1996, 61, 3849.

### EXAMPLE 1

N-(4-{[({Imino[(1-naphthylmethyl)amino]methyl}amino)carbonyl]amino}phenyl)acetamide

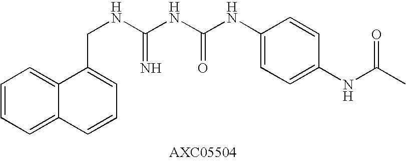

Compound 1 was prepared by the General Synthetic Method I above according to the following Specific Method:

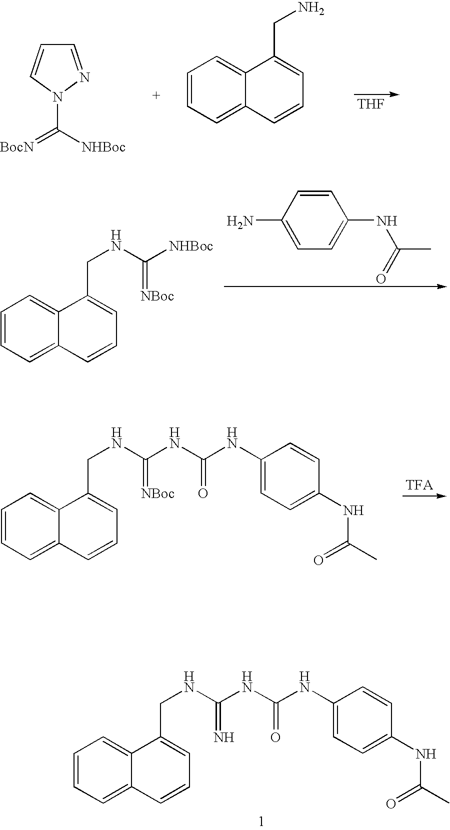

Preparation of tert-butyl-[(tert-butoxycarbonyl)amino][(1-naphthylmethyl) amino]methylidenecarbamate: Into a 250 mL round bottom flask was placed 1-naphthalenemethylamine (3.5 g, 22.2 mmol) and THF (100 mL). To this mixture at room temperature was added 1-H-pyrazole-1 (N,N′-bis(tert-butoxycarbonyl)carboxamidine (6.9 g, 22.2 mmol). The reaction mixture was stirred at room temperature for two hours. The mixture was concentrated and the residue was purified by silica gel column using eluting solvent hexane/ethyl acetate (18:1). The organic solvent was removed under reduced pressure to afford 7.5 g of the title compound. ¹HNMR (CDCl₃): δ1.43 (s, 9H), 1.55 (s, 9H), 5.07 (d, 2H), 7.41-7.55 (m, 4H), 7.82 (d, 1H), 7.88 (d, 1H), 8.048 (d, 1H), 8357 (t brd, 1H), 11.57 (s, 1H). MS (M+1): 400.5.

Preparation of tert-butyl-({[4-(acetylamino)anilino]carbonyl}amino)[(1-naphthylmethyl)amino]methylidenecarbamate: Into a 25 mL round bottom flask was placed tert-butyl-[(tert-butoxycarbonyl)amino][(1-naphthylmethyl)amino]methylidene carbamate (200 mg, 0.5 mmol), THF (5 mL) and 4′-amino acetanilide (75 mg, 0.5 mmol). The reaction mixture was heated to reflux overnight. The solvent was removed under reduced pressure. The residue was loaded on a silica gel column and eluted with hexane/ethyl acetate (1:1) to give 200 mg of the title compound. ¹HMR (CDCl₃): δ1.45 (s, 9H), 2.14 (s, 3H), 5.04 (d, 2H), 7.05 (brd, 2H), 7.41-7.59 (m, 8H), 7.80-7.90 (m, 2H), 8.06 (d, 1H), 8.52 (brd, 1H), 11.96 (s, 1H). MS: 476 (M+1).

Preparation of N-(4-{[({imino[(1-naphthylmethyl)amino]methyl}amino)carbonyl]amino}phenyl)acetamide (1): Into a 25 mL round bottom flask was placed tert-butyl-({[4-(acetylamino)anilino]carbonyl}amino)[(1-naphthylmethyl)amino]methylidene carbamate (200 mg, 0.42 mmol) and 3 mL of 50% mixture of TFA/CH₂Cl₂. The solution was stirred at room temperature for 1 hour. TFA and dichloromethane were removed by evaporation. The residue was dissolved in 3.0 M HCl in MeOH. The solvent was removed under reduced pressure to afford 166 mg of white solid hydrogen chloride salt of 1. ¹HNMR (DMSO-d₆): δ2.01 (s, 3H), 5.04 (s, 2H), 7.35 (d, 2H), 7.52-7.65 (m, 6H), 7.92-8.06 (m, 3H), 8.79 (brd, 2H), 9.49 (brd, 1H), 9.91 (s, 1H), 10.29 (brd, 1H). MS (APCI); 376.0 (M+1), (High resolution): 376.1788 (M+1).

Other intermediates of formula B useful in preparing the compounds of formula D by the method of Example 1 were synthesized as above. Such compounds include the following:

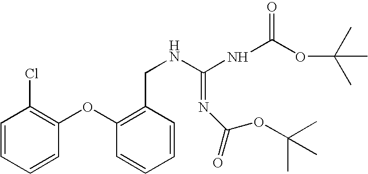

tert-butyl-[(tert-butoxycarbonyl)amino]{[2-(2-chlorophenoxy)benzyl}amino}methylidenecarbamate, ¹HNMR (CDCl₃): δ1.46 (s, 9H), 1.50 (s, 9H), 4.84 (s, 2H), 6.7-6.8 (m, 1H), 6.99 (d, 2H), 7.11-7.18 (m, 3H), 7.32 (t, 2H), 8.5 (t, 1H), 11.4 (s, 1H). MS: 476 (+1).

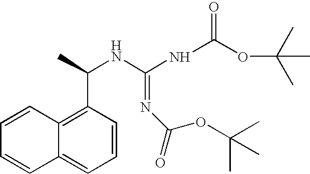

tert-butyl-[(tert-butoxycarbonyl)amino]{[(1R)-1-(1-naphthyl)ethyl]amino}methylidenecarbamate, ¹HNMR (CDCl₃): δ1.47 (s, 9H, 1.48 (s, 9H), 1.68 (d, 3H), 6.16 (m, 1H), 7.46-7.55 (m, 4H), 7.79 (d, 1H), 7.86 (d, 1H), 8.21 (d, 1H), 8.9 (d, 1H), 11.57 (s, 1H).

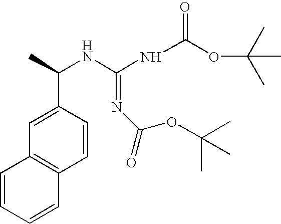

tert-butyl-[(tert-butoxycarbonyl)amino]{[(1R )-1-(2-naphthyl)ethyl]amino}methylidenecarbamate, ¹HNMR (CDCl₃): δ1.49 (d, 18H), 1.61 (d, 3H), 5.6 (m, 1H), 7.44-7.49 (m, 3H), 7.78-7.84 (m, 4H), 8.8 (d, 1H), 11.55 (s, 1H). MS: 414 (M+1)

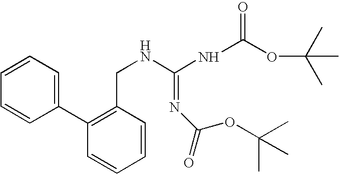

tert-butyl-[([1,1′-biphenyl}-2-ylmethyl)amino][(tert-butoxycarbonyl)amino]methylidenecarbamate, ¹HNNR (CDCl₃): δ1.47 (s, 9H), 1.51 (s, 9H), 4.56 (d, 2H), 7.27-7.43 (m, 9H), 8.35 (t, 1H), 11.45 (s, 1H). MS: 426.1 (M+1).

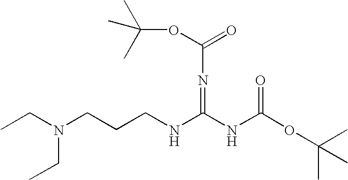

tert-butyl-[(tert-butoxycarbonyl)amino]{[3-(diethylamino)propyl]amino}methylidenecarbamate, ¹HNMR (CDCl₃): δ1.01 (t, 6H), 1.48 (s, 9H), 1.50 (s, 9H), 1.67-1.72 (m, 2H), 2.46-2.54 (m, 6H), 3.43-3.50 (m, 2H), 8.67 (s, 1H), 11.48 (s, 1H). MS: 373.24 (M+1).

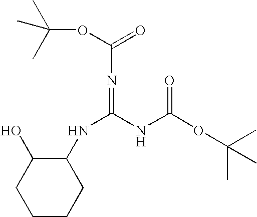

tert-butyl-[(tert-butoxycarbonyl)amino][(2-hydroxycyclohexyl)amino]methylidenecarbamate, ¹HNMR (CDCl₃): δ1.22-1.41 (m, 4H), 1.46 (s, 9H), 1.49 (s, 9H), 1.67-1.73 (m, 2H), 1.9-2.08 (m, 2H), 3.41 (m, 1H), 3.84 (m, 1H), 5.49 (s, 1H) 842 (d, 1H), 11.49 (s, 1H). MS: 358.2 (M+1).

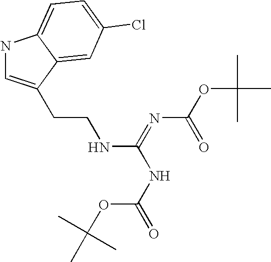

tert-butyl-[(tert-butoxycarbonyl)amino]{[2-(5-chloro-1H-indol-3-yl)ethyl]amino}methylidenecarbamate, ¹HNMR (CDCl₃): δ1.48 (s, 9H), 1.50 (s, 9H),2.97 (t, 2), 3.69 (q, 2H), 7.03 (d, 1H), 7.12 (dd, 1H), 7.26 (s, 1H), 7.55 (d, 1H), 8.42 (s, 1H), 8.50 (s, 1H), 10.8 (s, 1H). MS: 437.11 (M+1).

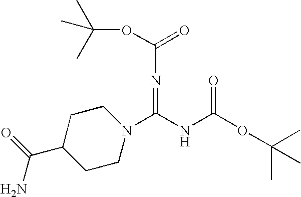

tert-butyl-[4-(aminocarbonyl)-1-piperidinyl][(tert-butoxycarbonyl)amino]methylidenecarbamate, ¹HNMR (CDCl₃): δ1.47 (s, 9H), 1.50 (s, 9H), 1.69-1.97 (m, 4H), 2.37-2.44 (m, 1H), 3.02 (t, 2H), 4.13-4.17 (m, 2H), 5.55 (s_{brd}, 2H), 10.18 (s, 1H). MS: 371.20 (M+1).

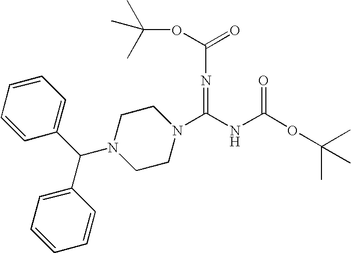

tert-butyl-(4-benzhydryl-1-piperazinyl)[tert-butoxycarbonyl)amino]methylidenecarbamate, ¹HNMR (CDCl₃): δ1.47 (d, 18H), 2.42-2.46 (m, 4H), 3.59 (s_{brd}, 4H), 4.25 (s, 1H), 7.18-7.29 (m, 6H), 7.39-7.42 (m, 4H), 10.17 (s, 1H).

### EXAMPLE 2

4-({[({Imino[(1-naphthylmethyl)amino]methyl}amino)carbonyl]amino}methyl)piperidine (2).

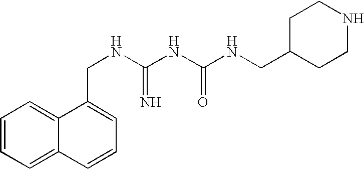

Compound 2 was prepared by General Synthetic Method I above.

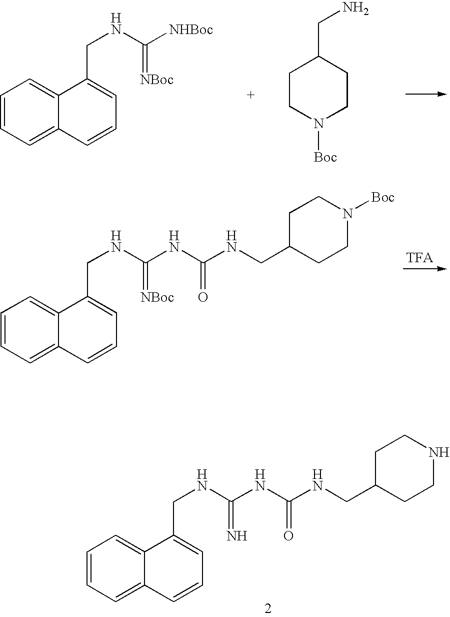

Preparation of tert-butyl 4-{9,9-dimethyl-5-[(1-naphthylmethyl)amino]-3,7-dioxo-8-oxa-2,4,5-triaza-5-decen-1-yl}-1-piperidinecarboxylate: Into a 250 mL round bottom flask was placed tert-butyl-[(tert-butoxycarbonyl)amino][(1-naphthyl methyl)amino]methylidenecarbamate (5.0 g, 15 mmol), 1-tert-butoxycarbonyl-4-aminomethylpiperidine (3.0 g, 15 mmol), which had been prepared according to Carceller, E. et. al; (J. Med. Chem. 1996, 39, 487-493), and THF (150 mL). The reaction mixure was heated to reflux overnight. The solvent was removed under reduced pressure. The residue was loaded on a silica gel column and eluted with hexane/ethyl acetate (from 5:1 to 2:1) to give 5.7 g of the title compound. ¹HNMR (CDCl₃): δ1.10-1.23 (m, 3H), 1.44 (s, 18H), 1.51-1.73 (m 2H),2.68 (t, 2H), 3.01-3.09 (m, 2H), 3.96-4.19 (m, 2H), 5.01 (d, 2H), 5.37 (t, 1H), 7.36-7.57 (m, 5H), 7.79-7.90 (m, 2H), 8.02-8.05 (m, 1H), 8.55 (t, 1H), 12.16 (s, 1H).

Preparation of 4-({[({imino[(1-naphthylmethyl)amino]methyl}amino)carbonyl]amino}methyl)piperidine (2): A 500 mL round bottom flask was charged with tert-butyl 4-{9,9-dimethyl-5-[(1-naphthylmethyl)amino]3,7-dioxo-8-oxa-2,4,5-triaza-5decen-1-yl}-1-piperidinecarboxylate (5.7 g) and 100 mL of 50% mixture of TFA/CH₂Cl₂. The solution was stirred at room temperature for 8 hours. TFA and dichloromehane were removed by evaporation. The residue was dissolved in 1.0 M HCl in MeOH. The solvents were removed under reduced pressure to afford 2.9 g of 2 as a white solid hydrogen chloride salt. ¹HNMR (DMSO-d₆): δ1.30-1.47 (m, 2H), 1.76-1.99 (m, 3H), 2.69-2.82 (m, 2H), 3.03 (brd, 2H), 3.17-3.25 (brd, 2H), 5.05 (s, 2H), 7.52-7.63 (m, 4H), 7.91-8.06 (m, 3H), 8.2 (brd, 1H), 8.94 (brd, 3H), 9.00 (brd, 3H), 10.8 (brd, 1H). MS (APCI): 340.1 (M+1).

### EXAMPLE 3

N-Acetyl-N′-(1-naphthylmethyl)guanidine (3)

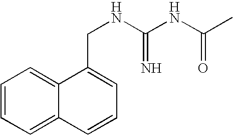

Compound 3 was prepared by General Synthetic Method II above.

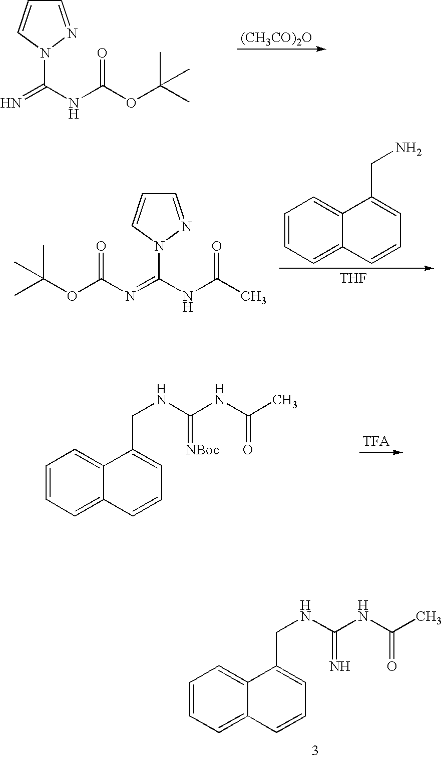

Preparation of tert-butyl(acetylimino)(1H-pyrazol-1-yl)methylcarbamate: A dried 250 mL round bottom flask was charged with 1-H-pyrazolo-1-(N,N′-bis(t-butoxycarbonyl)carboxamidine (95.0 g, 23.8 mmol), diisopropylethylamine (6.2 mL, 35.7 mmol) and dry THF(100 mL). To this solution was added acetic anhydride (3.7 g, 35.7 mmol). The solution was heated to reflux overnight under N₂. The reaction mixture was cooled to room temperature and solvent was removed. The residue was purified on silica gel chromatography eluted with hexane/ethyl acetate (9:1 to 4:1) to afford the title compound (1.1 g). ¹HNMR (CDCl₃): δ1.55 (s, 9H), 2.29 (s, 3H), 6.45 (t, 1H), 7.26 (s, 1H), 7.65 (s, 1H), 8.29 (s, 1H). MS: 252.9 (M+1).

Preparation of tert-butyl-(acetylamino)[(1-naphthylmethyl)amino]methylidenecabamate: To a solution of tert-butyl(acetylimino)(1H-pyrazol-1-yl)methylcarbamate (500 mg, 2.0 mmol) in THF (5 mL) was added 1-naphthylmethyl amine (312 mg, 2.0 mmol). The solution was stirred at room temperature overnight. The solvent was removed and the residue was purified by silica gel column chromatography using hexane/ethyl acetate(5:1 to 2:1) as eluant to give the title compound (412 mg). ¹HNMR (CDCl₃): δ1.57 (s, 9H), 2.18 (s, 3H), 5.07 (d, 2H), 7.42-7.55 (m, 4H), 7.80-7.86 (m, 2H), 7.98 (d, 1H), 9.1 (s, 1H), 12.48 (s, 1H). MS: 342 (M+1).

Preparation of N-acetyl-N′-(1-naphthylmethyl)guanidine (3): Tert-butyl-(acetylamino)[(1-naphthylmethyl)amino]methylidenecabamate (380 mg) was dissolved in a solution of 50% TFA/dichloromethane (5 mL). The reaction contents were stirred at room temperature for 1 hour. The reaction solvent and excess amount of TFA were removed under reduced pressure. The residue was dissolved in 2N HCl/MeOH. The solution was concentrated to afford compound 3 (242 mg). ¹HNMR (DMSO-d₆): δ2.16 (s, 3H), 5.04 (d, 2H), 7.47-7.62 (m, 4H), 7.91-8.03 (m, 3H), 8.74 (brd, 1H), 9.07 (brd, 1H), 9.38 (s, 1H), 12.06 (s, 1H). MS (APCI): 241.9 (M+1).

### EXAMPLE 4

Methylimino[(1-naphthylmethyl)amino]methylcarbamate (4)

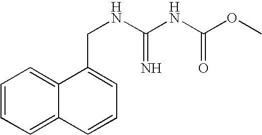

Compound 4 was prepared by General Synthetic Method III above.

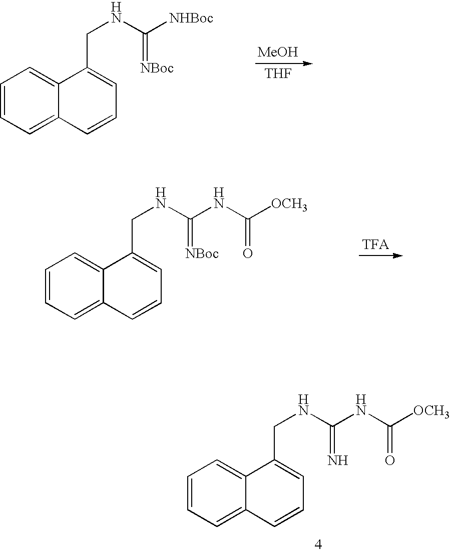

Preparation of methyl[(tert-butoxycarbonyl)imino][(1-naphthylmethyl)amino]methylcarbamate: A solution of tert-butyl-[(tert-butoxycarbonyl)amino][(1-naphthyl methyl) amino]methylidenecarbamate (100 mg, 0.25 mmol) and methanol (1 mL) in THF (5 mL) was heated to reflux overnight. The reaction mixture was concentrated. The residue was purified using silica gel column chromatography, eluting with hexane/ethyl acetate (4:1), to give the title compound (40 mg). ¹HNMR (CDCl₃): δ1.4 (s, 9H), 3.75&3.8 (s, 3H),5.05&5.15 (d, 2H), 7.4-7.6 (m, 4H), 7.9-7.94 (m, 2H), 8.01-8.1 (m, 1H), 8.3&8.6 (s, 1H), 11.8&12.2 (s, 1H).

Preparation of Methylimino[(1-naphthylmethyl)amino]methylcarbamate (4): The product from the previous step, methyl [(tert-butoxycarbonyl)imino][(1-naphthylmethyl) amino]methylcarbamate (40 mg), was dissolved in a solution of 50% TFA/dichloromethane (2 mL). The reaction contents were stirred at room temperature for 1 hour. The reaction solvent and excess amount of TFA were removed on a rotary evaporator. The residue was dissolved in 3N HCI/MeOH. The solution was concentrated to afford compound 4. ¹HNMR (DMSO-d₆): δ3.77 (s, 3H), 5.04 (d, 2H), 7.41-7.62 (m, 4H), 7.90-8.02 (m, 3H), 8.65 (brd, 1H), 9.09 (brd, 1H), 9.21 (t_{brd}, 1H), 11.58 (s, 1H). MS (APCI): 258 (M+1).

### EXAMPLE 5

N-{[4-(2-Methoxybenzyl)-1-piperazinyl]carbonyl}-N′-(1-naphthylmethyl)guanidine (5)

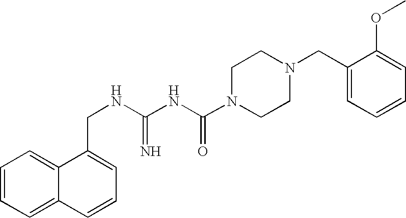

Compound 5 was made by General Synthetic Scheme IV above.

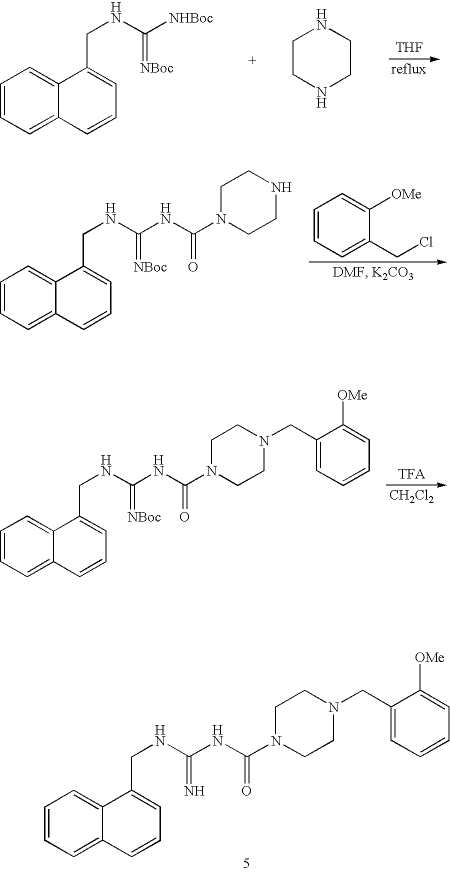

Preparation of tert-butyl-[(1-naphthylmethyl)amino][(1-piperazinylcarbonyl) amino]methylidenecarbamate: A solution of tert-butyl-[(tert-butoxycarbonyl) amino][(1-naphthylmethyl)amino]methylidenecarbamate (4.0 g, 10 mmol) and piperazine (1.2 g, 10 mmol) in THF (150 mL) was heated to reflux for 8 hours. The solvent was removed under reduced pressure. The residue was purified by silica gel column eluted with dichloromethane/methanol (8:1 to 2:1) to give 3.2 g of the title compound. ¹HNMR (CDCl₃): δ1.42 (s, 9H), 2.38-2.46 (m, 2H), 2.77-2.84 (m, 2H), 3.56-3.74 (m, 4H), 5.00 (s, 2H), 7.23-7.88 (m, 7H), 8.39 (s, 1H), 12.32 (S, 1H). MS: 412 (M+1).

Preparation of tert-butyl-({[4-(2-methoxybenzyl)-1-piperazinyl]carbonyl}amino)[(1-naphthylmethyl)amino]methylidenecarbamate: To a solution of the product from above (tert-butyl-[(1-naphthylmethyl)amino][(1-piperazinylcarbonyl)amino]methylidenecarbamate, 70 mg, 0.17 mmol) in DMSO (2 mL) was added K₂CO₃ (47 mg, 0.34 mmol) and 2′-chloroethyl-2-methoxybenzene (27 mg, 0.17 mmol). The solution was stirred at room temperature for 30 minutes. The reaction mixture was extracted with ethyl acetate. The organic layer was washed with water (3 times) and brine, dried over MgSO₄ and concentrated. The residue was purified by silica gel column chromatography eluted with Cff₂Cl₂/MeOH (99:1) to give compound the title compound. ¹HNMR (CDCl₃): δ1.41 (s, 9H), 2.45 (d, 4H), 3.52-3.61 (m, 4H), 3.80 (m, 5H), 4.99 (d, 2H), 6.84-6.94 (m, 2H), 7.18-7.54 (m, 6H), 7.78-7.88 (m, 2H), 8.05-8.08 (m, 1H), 8.35 (t, 1H), 12.34 (s, 1H),

Preparation of N-{[4-(2-methoxybenzyl)-1-piperazinyl]carbonyl}-N′-(1-naphthyl methyl)guanidine (5): The product (tert-butyl-({[4-(2-methoxybenzyl)-1-piperazinyl]carbonyl}amino)[(1-naphthylmethyl)amino]methylidenecarbamate) prepared in the previous step was treated with 50% TFA/CH₂Cl₂ (according to step 3 of Scheme 1) and purified by HPLC. 42 mg of (5) was obtained. ¹HNMR (DMSO-d₆): δ3.53-3.66 (m, 4H), 4.03 (s, 3H), 4.08 (brd, 4H), 4.48 (s, 2H), 5.23 (d, 2H), 7.22 (t, 1H), 7.32 (d, 1H), 7.62-7.84 (m, 6H), 8.10-8.24 (m, 3H), 9.18 (s brd, 2H), 9.86 (t brd, 1H), 11.09 (brd, 1H), 11.45 (s, 1H). MS (APCI) 432.1 (M+1).

### EXAMPLE 6

1-Benzyl-4-{[({imino[(1-naphthylmethyl)amino]methyl}amino)carbonyl]amino}piperidine (6)

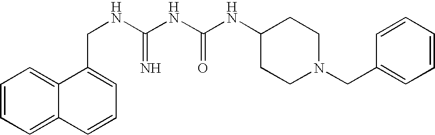

Compound 2 was treated with benzyl chloride under basic conditions to afford 1-benzyl-4-{[({imino[(1-naphthylmethyl)amino]methyl}amino)carbonyl]amino}piperidine (6), ¹HNMR (DMSO-d₆): δ1.83-2.01 9m, 4H), 2.94-3.05 (m, 2H), 3.16-3.33 (m, 2H), 3.70 (m, 1H), 4.23 (d, 2H), 5.01 (d, 2H), 7.44-7.64 (m, 9H), 7.90-8.03 (m, 3H), 8.2 (brd, 1H), 8.74 (brd, 2H), 9.5 (brd, 1H), 10.1 (brd, 1H), 10.75 (brd, 1H). MS (APCI): 416 (M+1).

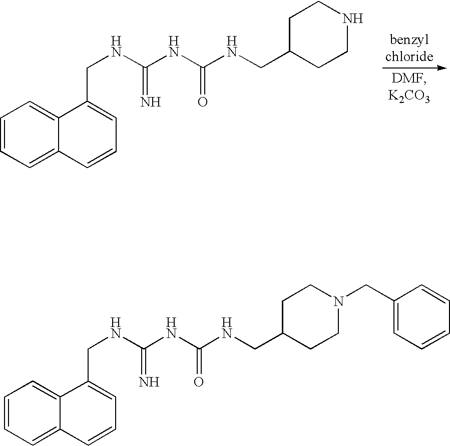

### EXAMPLE 7

N-[(4-Benzyl-1-piperazinyl)carbonyl]-N′-(1-naphthylmethyl)guanidine (7)

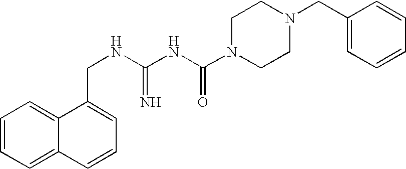

Compound 7 was prepared by General Synthetic Method VI above.

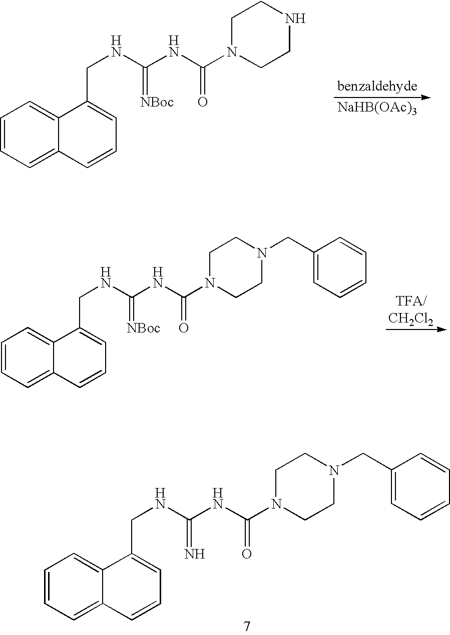

Preparation of tert-butyl-{[(4-benzyl-1-piperazinyl)carbonyl]amino}[(1-naphthylmethyl)amino]methylidenecarbamate: Into a 100 mL round bottom flask was placed tert-butyl-[(1-naphthylmethyl)amino][(1-piperazinylcarbonyl)amino]methylidenecarbamate (Example 4 above; 1 g, 2.4 mmol), benzaldehyde (254 mg, 2.4 mmol), sodium triacetoxyborohydride (1.3 g, 6 mmol) and acetonitrile (50 mL). The reaction mixture was stirred at room temperature under nitrogen for 6 hours. The reaction was quenched by 20 mL of saturated sodium bicarbonate. The crude mixture was poured in water and extracted with ethyl acetate two times. The combined organic layers were washed with brine, dried over magnesium sulfate and concentrated. The residue was purified by silica gel column chromatography eluting with 2%-5% methanol in methylene chloride. 1.1 g of the title compound was obtained. ¹HNMR (CDCl₃): δ1.41 (s, 9H), 2.36-2.37 (t, 2H), 2.41-2.44 (t, 2H), 3.51 (s, 2H), 3.60 (t, 2H), 3.79 (t, 2H), 4.99 (d, 2H), 7.26-7.32 (m, 5H), 7.42-7.53 (m, 4H), 7.80 (d, 1H), 7.86-7.89 (m, 1H), 8.04 (m, 1H), 8.4 (t, 1H), 12.34 (s, 1H). MS: 502 (M+1).

Preparation of N-[(4-benzyl-1-piperazinyl)carbonyl]-N′-(1-naphthylmethyl) guanidine (7): The product from the previous step, tert-butyl-{[(4-benzyl-1-piperazinyl)carbonyl]amino})([-naphthylmethyl)amino]methylidenecarbamate, was treated with 50% TFA/CH₂Cl₂ and purified by HPLC. 777 mg of 7 was obtained. ¹HNMR (DMSO-d₆): δ3.10 (brd, 2H), 3.44 (brd, 2H), 4.31 (brd, 8H), 5.04 (d, 2H), 7.44-7.62 (m, 9H), 7.92-8.04 (m, 3H), 8.97 (s, 2H), 9.64 (s, 1H), 11.26 (s, 1H), 11.5 (brd, 1H). MS (APCI): 402.1 (M+1), High resolution): 402.2280 (M+1).

### EXAMPLES 8-87

Compounds 8 through 87 were prepared by straightforward modifications of the methods described in general above and in detail in Examples 1-7.

### EXAMPLE 8

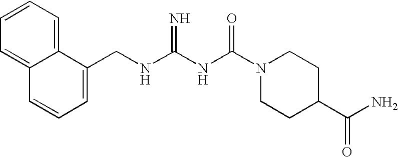

1-[({imino[(1-naphthylmethyl)amino]methyl}amino)carbonyl]-4-piperidinecarboxamide, ¹HNMR(DMSO-d₆): δ1.48 (m, 2H),1.74 (m, 2H), 2.35 (m, 1H), 2.94 (brd, 2H), 3.90 (brd, 2H), 4.16 (dd, 2H), 5.01 (d, 2H), 7.52 (d, 1H), 7.53 (d, 1H), 7.57-7.65 (m, 2H), 7.92-7.95 (m, 1H), 8.01 (d, 1H), 8.02 (d, 1H, 8.8 (brd, 1H), 9.1 (brd, 1H), 9.63 (s, 1H), 10.64 (s,1H). MS (APCI): 354 (M+1) (High resolution): 354.1930 (M+1).

### EXAMPLE 9

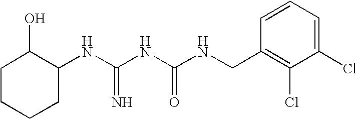

1,2-Dichloro-3-{[({[[(2-hydroxycyclohexyl)amino](imino)methyl]amino}carbonyl)amino]methyl}benzene, ¹HNMR (CD₃OD): δ1.3 (m, 4H), 1.7 (m, 2H), 1.99 (m, 2H), 3.35 (m, 2H), 4.45 (s, 2H), 7.45-7.6 (m, 2H), 7.7 (d, 1H). MS (APCI): 359.1 (M+1)

### EXAMPLE 10

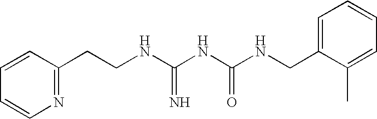

2-(2-{[Imino([{(2-methylbenzyl)amino]carbonyl}amino)methyl]amino}ethyl)pyridine, ¹HNMR (CD₃OD): δ2.25 (s, 3H), 3.6 (t, 2H), 4.25 (s, 2H), 7.05 (s, 3), 7.19 (d, 1H), 7.6-7.7 (m, 2H), 8.1 (t, 1H), 8.65 (d, 1H). MS (APCI): 312.2 (M+1).

### EXAMPLE 11

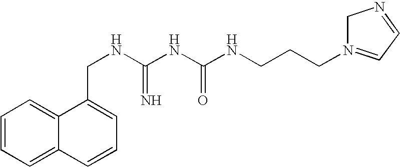

1-(3-{[({Imino[(1-naphthylmethyl)amino]methyl}amino)carbonyl]amino}propyl0-2H-1⁵-imidazole, ¹HNMR (DMSO-d₆): δ2.0 (m, 2H), 3.09 (brd, 2H), 4.25 (t, 2H), 5.03 (s, 2H), 7.49-7.63 (m, 4H), 7.69 (s, 1H), 7.83 (s, 1H), 7.92 (d, 1H), 7.98-8.05 (m, 3H), 8.85 (brd, 2H), 9.22 (s, 1H), 9.53 (brd, 1H), 10.80 (brd, 1H). MS (APCI ):351.2 (M+1).

### EXAMPLE 12

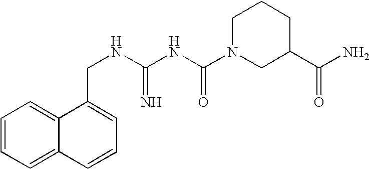

1-[({Imino[(1-naphthylmethyl)amino]methyl}amino)carbonyl]-3-piperidine carboxamide, ¹HNMR (DMSO-d₆): δ1.38 (m, 1H), 1.57-1.68 (m, 2H), 1.85-1.88 (m, 1H), 2.3 (brd, 1H), 2.93 (brd, 2H), 3.6 (d, 2H), 4.13 (d, 2H), 5.03 (d, 2H), 7.53 (d, 1H), 7.54 (d, 1H), 9.1 (brd, 1H), 9.7 (brd, 1H), 10.7 (brd, 1H). MS (APCI): 354.1 (M+1).

### EXAMPLE 13

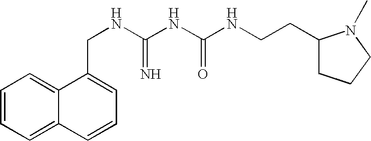

2-(2-{[{Imino[(1-naphthylmethyl)amino]methyl}amino)carbonyl]amino}ethyl)-1-methylpyrrolidine, ¹HNMR (DMSO-d₆): δ1.64 (m, 1H), 1.75-2.00(m, 3H), 2.1 (m, 1H), 2.27 (m, 1H), 2.74 (d, 3H), 2.99 (m, 1H), 3.19 (brd, 3H), 3.49 (m, 1H), 5.01 (brd, 2H), 7.52-7.54 (d, 2H), 7.57-7.64 (m, 2H), 7.93 (d, 1H), 8.00 (d, 1H), 8.03 (d, 1H), 8.79 (brd, 2 H), 9.5 (brd, 1H), 10.63 (brd). MS (APCI): 354 (M+1).

### EXAMPLE 14

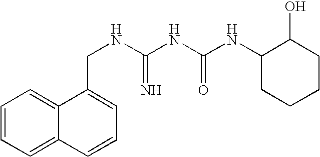

1-({[({[(2-Hydroxycyclohexyl)amino]carbonyl}amino)(imino)methyl]amino}methyl)naphthalene, ¹HNMR (DMSO-d₆): δ1.19 (brd, 4H), 1.59 (brd, 2H), 1.83 (brd, 2H), 3.27 (brd, 2H), 5.01 (s, 2H), 7.52 (d, 2H), 7.60 (m, 2H), 7.73 (brd, 1H), 7.93 (d, 1H), 8.00 (d, 1H), 8.04 (d, 1H), 8.78 (brd, 2H), 9.56 (brd, 1H), 10.25 (brd, 1H). MS (APCI): 341.2 (+1).

### EXAMPLE 15

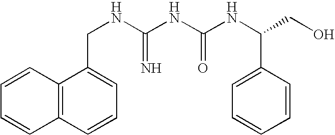

1-({[[({[(1S)-2-Hydroxy-1-phenylethyl]amino}carbonyl)amino](imino)methyl]amino}methyl)naphthalene, ¹HNMR (DMSO-d₆): δ3.52-3.65 (m, 2H), 4.72 (m, 1H), 4.98 (s, 2H), 5.03 (brd, 1H), 7.24-7.63 (m, 9H), 7.92 (d, 1H), 7.97 (d, 1H), 7.98 (d, 1H), 8.35 (brd, 1H0, 8.75 (brd, 1H), 9.5 (brd, 1H), 10.2 (brd, 1H). MS (APCI): 363.0 (M+1), (high resolution) 363.1831 (M+1).

### EXAMPLE 16

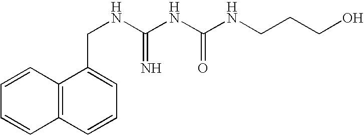

1-({[({[(3-Hydroxypropyl)amino}carbonyl}amino)(imino)methyl]amino}methyl)naphthalene, ¹HNMR (DMSO-d₆): δ1.81-1.91 (m, 1H), 2.32 (s, 2H), 2.93 (brd, 1H), 3.36 (t, 2H), 4.22 (s, 2H), 5.05 (brd, 1H), 7.25 (d, 2H), 7.26-7.62 (m, 3H), 7.92-8.00 (m, 2H), 8.7 (brd, 0.5H), 9.1 (brd, 0.5H), 10.6 (brd, 0.5H), 11.6 (brd, 0.5). MS (APCI): 301 (M+1).

### EXAMPLE 17

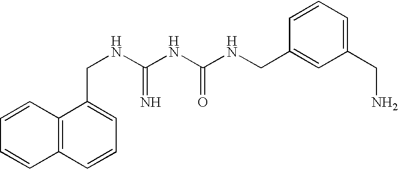

1-({[[({[3-(Aminomethyl)benzyl]amino}carbonyl)amino](imino)methyl]amino}methyl)naphthalene, ¹HNMR (DMSO-d₆): δ3.98 (d, 2H), 4.32 (d, 2H), 5.03 (s, 2H), 7.31-7.62 (m, 8H), 7.90-8.02 (m, 3H), 8.37 (brd, 3H), 8.76 (brd, 1.5H), 9.4 (brd, 0.5H), 10.8 (brd, 1H). MS (APCI) 362.2 (M+1).

### EXAMPLE 18

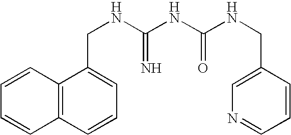

3-({[({Imino[(1-naphthylmethyl)amino]methyl}amino)carbonyl]amino}methyl)pyridine, ¹HNMR (DMSO-d₆): δ4.47 (s, 21H), 4.99 (s, 2H), 7.49-7.64 (m, 5H), 7.90-8.02 (m, 4H), 8.2-8.59 (brd, 4H), 8.77 (brd, 2H), 9.39 (brd, 1H), 10.8 (brd, 1H). MS (APCI): 334.0 (M+1).

### EXAMPLE 19

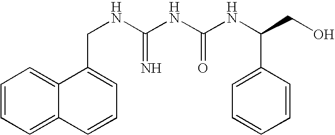

1-({[[({[(1R)-2-Hydroxy-1-phenylethyl]amino}carbonyl)amino](imino)methyl]amino}methyl)naphthalene, ¹HNMR (DMSO-d₆): δ3.37 (brd, 1H), 3.50-3.65 (m, 2H), 4.73 (dd, 1H), 5.01 (s, 2H), 7.24-7.27 (m, 1H), 7.31-7.33 (m, 4H), 7.49 -7.61 (m, 4H), 7.90-8.03 (m, 3H), 8.36 (brd, 1H), 8.73 (brd, 1.5H), 9.4 (brd, 0.5H), 10.4 (brd, 1H). MS (APCI): 363.1 (M+1), (High resolution): 363.1829 (M+1)

### EXAMPLE 20

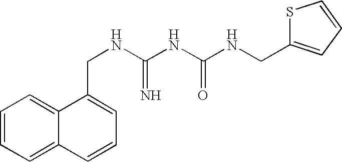

2-({[({Imino[(1-naphthylmethyl)amino]methyl}amino)carbonyl]amino}methyl)thiophene, ¹HNMR (DMSO-d₆): δ4.47 (d, 2H), 5.02 (d, 2H), 6.95-7.01 (m, 2H), 7.4-7.64 (m, 5H), 7.90-8.04 (m, 3H), 8.4 (brd, 1H), 8.8 (brd, 2H), 9.4 (brd, 1H), 10.6 (brd, 1H). MS (APCI): 339 (M+1)

### EXAMPLE 21

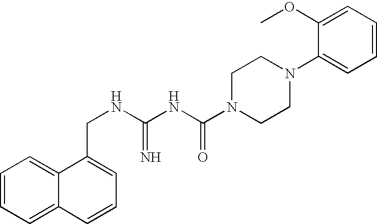

N-{[4-(2-Methoxyphenyl)-1-piperazinyl]carbonyl}-N′-(1-naphthylmethyl)guanidine, ¹HNMR (DMSO-d₆): δ3.21 (m brd, 4H), 3.84 (s, 3H), 3.87 (brd, 4H), 5.06 (d, 2H), 6.9-7.2 (m, 4H), 7.50-7.7 (m, 4H), 7.90-8.09 (m, 3H), 9.1 (brd, 2H), 9.9 (t_{brd}, 1H), 11.2 (s, 1H). MS (APCI): 418.1 (M+1), (High resolution): 418.2245 (M+1)

### EXAMPLE 22

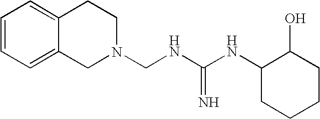

N-[3,4-Dihydro-2(1H)-isoquinolinyl(imino)methyl]-N′-(2-hydroxycyclohexyl)urea, ¹HNMR (DMSO-d₆): δ1.11 (brd, 4H), 1.41-1.65 (m, 2H), 1.78 (brd, 1H), 2.06+(br, 1H), 2.79 (m, 2H), 3.69 (m, 2H), 4.16 (m, 2H), 4.65 (s, 2H), 7.10 (s, 4H).

### EXAMPLE 23

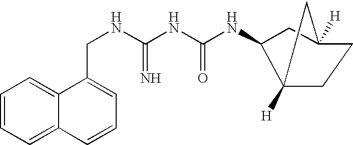

1-({[({[(1S,2S,4R)Bicyclo[2.2.1]hept-2-ylamino]carbonyl}amino) (imino)methyl]amino}methyl)naphthalene, ¹HNMR (DMSO-d₆): δ1.03-1.45 (m, 7H), 1.61-1.67 (m, 1H), 2.1 (s, 1H), 2.22 (s, 1H), 3.50 (brd, 1H), 5.02 (s, 2H), 7.50-7.61 (m, 4H), 7.90-8.04 (m, 3H), 8.69 (brd, 2H), 9.45 (brd, 1H), 10.2 (brd, 1H). MS (APCI 337.1 (M+1).

### EXAMPLE 24

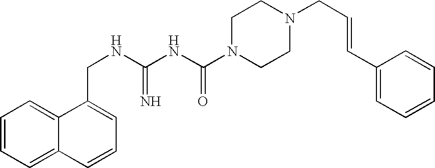

N-(1-Naphthylmethyl)-N′-({4-[(E)-3-phenyl-2-propenyl]-1-piperazinyl}carbonyl)guanidine, ¹HMR (DMSO-d₆): δ3.10 (brd, 2H), 3.40-3.56 (m, 4H), 3.90 (d, 2H), 4.45 (brd, 2H), 5.05 (d, 2H), 6.40 (m, 1H), 6.83 (d, 1H), 7.32-7.62 (m, 9H), 7.90-8.04 (m, 3H), 9.01 (brd, 2H), 9.69 (brd, 1H), 11.33 (brd, 1H), 11.4 (brd, 1H). MS (APCI): 428.1 (M+1), (High resolution): 428.2462 (M+1).

### EXAMPLE 25

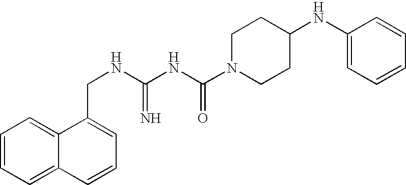

N-[(4-Anilino-1-piperidinyl)carbonyl]-N-(1-naphthylmethyl)guanidine, ¹HNMR (DMSO-d₆): δ1.57 (brd, 2H), 1.94 (d, 2H), 3.02 (brd, 2H), 3.64 (brd, 1H), 4.27 (brd, 2H), 5.02 (d, 2H), 7.23 (brd, 3H), 7.38 (brd, 2H), 7.50-7.65 (m, 4H), 7.91-8.04 (m, 3H), 8.94 (brd, 2H), 9.71 (brd, 1H), 10.97 (brd, 1H). MS (APCI): 402.3 (M+1), (High resolution): 402.2362 (M+1).

### EXAMPLE 26

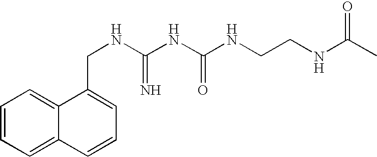

N-(2-{[({Imino[(1-naphthylmethyl)amino]methyl}amino)carbonyl]amino}ethyl)acetamide, ¹HNMR (DMSO-d₆): δ1.79 (s, 3H), 3.14 (s_{brd}, 4H), 5.01 )brd, 2H), 7.50-7.64 (m, 4H), 7.8 (brd, 1H), 7.91-8.04 9m, 3H0, 8.7 (brd, 2H), 9.4 (brd, 1h0, 10.5 (brd, 1H). MS (APCI): 328.1 (M+1).

### EXAMPLE 27

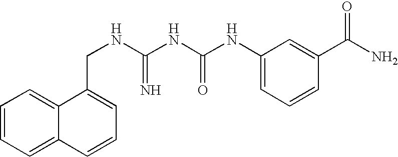

3-{[({Imino[(1-naphthylmethyl)amino]methyl}amino)carbonyl]amino}benzamide, ¹HNMR (DMSO-d₆): δ5.07 (s, 2H), 7.37-7.63 (m, 8H), 7.91-8.04 (m, 5H), 8.93 (brd, 2H), 9.5 (brd, 1H), 10.5 (brd, 2H).

### EXAMPLE 28

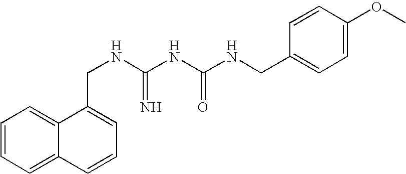

1-({[Imino({[(4-methoxybenzyl)amino]carbonyl}amino)methyl]amino}methyl)naphthalene, ¹HNMR (DMSO-d₆): δ3.72 (s, 3H), 4.23 (d, 2H), 5.01 (d, 2H), 6.85 (d, 2H), 7.18 (d, 2H), 7.46-7.64 (m, 4H), 7.91-8.04 (m, 3H), 8.26 (brd, 1H), 8.74 (brd, 1H), 9.39 (brd, 1H), 10.56 (brd, 1H). MS (APCI): 363.1 (M+1)

### EXAMPLE 29

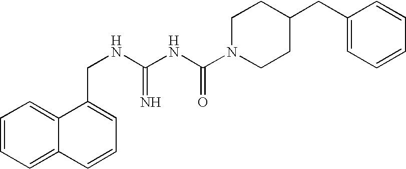

N-[(4-Benzyl-1-piperidinyl)carbonyl]-N′-(1-naphthylmethyl)guanidine, ¹HNMR (DMSO-d₆): δ0.81-0.86 (m, 1H), 1.03-1.14 (m, 2H), 1.2 (s brd, 1H), 1.57 (d, 2H), 1.72-1.77 (m, 1H), 2.81 (brd, 2H), 4.21 (brd, 2H), 5.00 (d, 2H), 7.14-7.29 (m, 5H), 7.50-7.61 (m, 4H), 7.90-8.03 (m, 3H), 8.83 (brd, 1H), 9.16 (brd, 1H), 9.75 (t brd, 1H), 10.78 (s, 1H). MS (APCI): 401.2 (M+1), (High resolution): 401.2353 (M+1).

### EXAMPLE 30

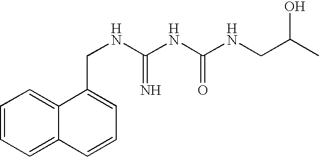

1({[({[(2-Hydroxypropyl)amino]carbonyl}amino)(imino)methyl]amino}methyl) naphthalene, ¹HNMR (DMSO-d₆): δ1.03 (d, 3H), 2.94-3.16 (m, 2H), 3.65-3.71 (m, 1H), 5.01 (d, 2H), 7.50-7.68 (m, 4H), 7.90-8.04 (m, 3H), 8.71 (brd, 2H), 9.4 (brd, 1H), 10.41 (s, 1H). MS (APCI): 301.1 (M+1).

### EXAMPLE 31

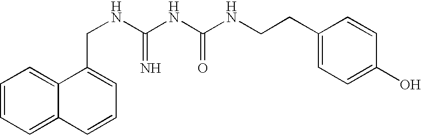

1-({[({[(4-Hydroxyphenethyl)amino]carbonyl}amino)(imino)methyl]amino}methyl) naphthalene, ¹HNMR (DMSO-d₆): δ2.63 (t, 2H), 3.25 (m, 2H), 3.6 (brd, 1H), 5.02 (d, 2H), 6.68 (d, 2H), 7.00 (d, 2H), 7.50-7.64 (m, 4H), 7.90-8.04 (m, 3H), 8.73 (s_{brd}, 2H) 9.19 (brd, 1H0, 9.4 (brd, 1H), 10.55 (s, 1H). MS (APCI): 363.1 (M+1).

### EXAMPLE 32

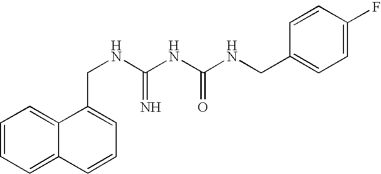

1-({[({[(4-Fluorobenzyl)amino]carbonyl}amino)(imino)methyl]amino}methyl)naphthalene, ¹HNMR (DMSO-d₆): δ4.29 (d, 2H), 5.02 (d, 2H), 7.15 (t, 2H), 7.31-7.36 (m, 2H), 7.50-7.62 (m, 4H), 7.92-8.04 (m, 3H), 8.36 (brd, 1H), 8.75 (brd, 2H), 9.40 (brd, 1H), 10.67 (brd, 1H). MS (APCI): 351.1 (M+1).

### EXAMPLE 33

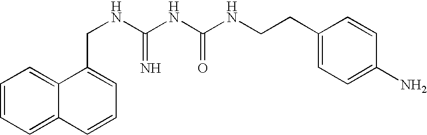

1-({[({[(4-Amninophenethyl)amino]carbonyl}amino)(imino)methyl]amino}methyl) naphthalene, ¹HNMR (DMSO-d₆): δ2.77 (t, 2H), 3.32-3.38 (m, 2H), 3.8 (brd, 2H), 5.02 (d, 2H), 7.26-7.34 (m, 4H), 7.50-7.62 (m, 4H), 7.90-8.01 (m, 3H), 8.74 (brd, 2H), 9.4 (brd, 1H), 10.2 (brd, 1H), 10.65 (brd, 2H). MS (APCI) 362.2 (M+1).

### EXAMPLE 34

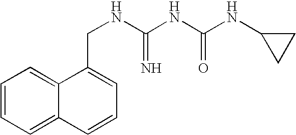

1-({[{[(Cyclopropylamino)carbonyl]amino}(imino)methyl]amino}methyl)naphthalene, ¹HMR (DMSO-d₆): δ0.47 (brd, 2H), 0.68 (brd, 2H), 2.51-2.58 (m, 1H), 5.00 (d, 2H), 7.50-7.64 (m, 4H), 7.90-8.03 (m, 3H), 8.769 (brd, 2H), 9.49 (brd, 1H), 10.1 (brd, 1H). MS (APCI): 283.1 (M+l), (High resolution): 283.1565 (M+1).

### EXAMPLE 35

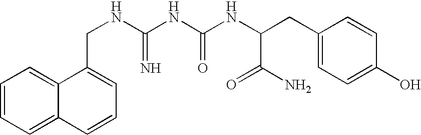

(2S)-3-(4-Hydroxyphenyl)-2-{[({imino[(1-naphthylmethyl)amino]methylamino) carbonyl]amino}propanamide, ¹HNMR (DMSO-d₆): δ2.85-2.95 (m, 2H), 4.05 (brd, 3H), 4.28 (m, 1H), 4.98 (d, 2H), 6.64-6.72 (m, 3H), 6.99-7.12 (m, 2H), 7.39-7.63 (m, 5H), 7.90-8.02 (m, 3H), 8.67 (brd, 2H), 9.3 (brd, 1H), 10.22 (s, 1H).

### EXAMPLE 36

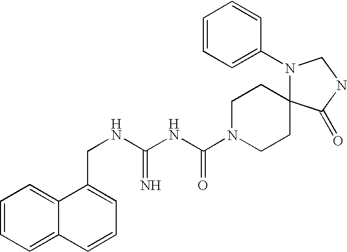

N-(1-Naphthylmethyl)-N′-[(4-oxo-1-phenyl-1,3,8-triazaspiro[4.5]dec-8yl)carbonyl]guanidine, ¹HNMR (DMSO-d₆): δ1.71 (d, 2H), 2.37-2.45 (m, 2H), 3.63 (t_{brd}, 2H), 4.19 (d, 2H), 5.04 (d, 2H), 6.74-6.80 (m, 3H), 7.20 (t, 2H), 7.49-7.65 (m, 4H), 7.92 (d, 1H), 7.99 (d, 1H), 8.04 (d, 1H), 8.79 (s, 1H), 9.01 (brd, 2H), 9.76 (s, 1H), 10.93 (s, 1H). MS (APCI): 457 (M+1).

### EXAMPLE 37

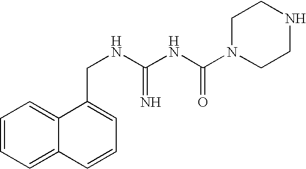

N-(1-Naphthylmethyl)-N′-(1-piperazinylcarbonyl)guanidine, ¹HNMR (DMSO-d₆): δ3.78-4.31 (m, 8H), 5.06 (d, 2H), 7.44-7.63 (m, 4H0, 7.91-8.05 (r, 3H), 9.03 (brd, 2H), 9.49 (s, 2H), 9.73 (s, 1H), 11.3 (S, 1H). MS (APCD: 312 (M+1).

### EXAMPLE 38

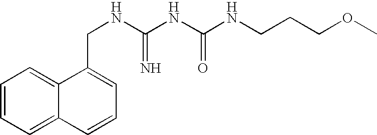

1-(3-Imino-5-oxo-10-oxa-2,4,6-triazaundec-1-yl)naphthalene, ¹HNMR (DMSO-d₆): δ1.66 (p, 2H), 3.11-3.17 (m, 2H), 3.21 (s, 3H), 3.34 (t, 2H), 5.03 (s, 2H), 7.48-7.63 (m, 4H), 7.90-8.04 (m, 3H), 8.72 (brd, 2H), 9.42 (brd, 1H), 10.65 (brd, 1H). MS (APCI): 315.1 (M+1), (High resolution) 315.1832 (M+1).

### EXAMPLE 39

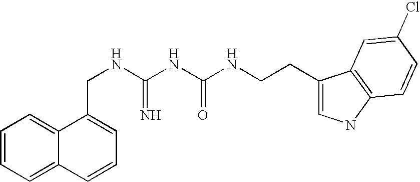

5-Chloro-3-(2-{[({imino[(1-naphthylmethyl)amino]methyl}amino)carbonyl]amino}ethyl)-1H-indole, ¹HNMR (DMSO-d₆): δ2.85 (t, 2H), 3.38 (q, 2H), 5.01 (d, 2H), 7.04-7.07 (dd, 1H), 7.27 (d, 1H), 7.34 (d, 1H), 7.50-7.64 (m, 5H), 7.90-8.04 (m, 3H), 8.72 (s, 2H), 9.4 (brd, 1H), 10.48 (s, 1H), 11.07 (s, 1H). MS (APCI): 420.0 (M+1).

### EXAMPLE 40

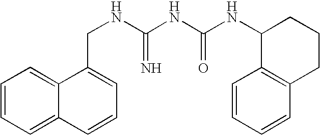

1-{[({Imino[(1-naphthylmethyl)amino]methyl}amino)carbonyl]amino}-1,2,3,4-tetrahydronaphthalene, ¹HNMR (DMSO-d₆): δ1.75-2.01 (m, 3H), 2.69-2.9 (m, 2H), 3.31 (s_{brd}, 2H), 5.03 (s, 2H), 6.96-7.63 (m, 8H), 7.91-8.04 (m, 3H), 8.75 (brd, 2H), 9.45 (brd, 1H), 10.58 (brd, 1H). MS (APCI): 373.10 (M+1).

### EXAMPLE 41

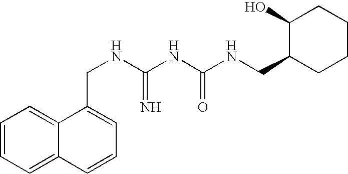

1-({[([({[(1S,2S)2-Hydroxycyclohexyl]methyl}amino)carbonyl]amino}(imino)methyl]amino}methyl)naphthalene, ¹HNMR (DMSO-d₆): δ1.16-1.38 (m, 5H), 1.48-1.66 (m, 4H), 2.98-3.10 (m, 2H), 3.74 (brd, 1H), 4.03 (brd, 1H), 5.01 (d, 2H), 7.50-7.64 (m, 4H), 7.90-8.04 (m, 3H), 8.68 (brd, 2H), 9.39 (brd, 1H), 10.49 (brd, 1H). MS (APCI): 355.1 (M+1), (High resolution): 355.2145 (M+1).

### EXAMPLE 42

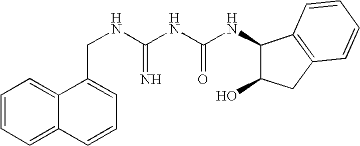

1-({[[({[(1S,2R)-2-Hydroxy-2,3-dihydro-1H-inden-1-yl]amino}carbonyl)amino](imino)methyl]amino}methyl)naphthalene, ¹HNMR (DMSO-d₆): δ2.78,2.87 (dd, 1H), 3.04,3.19 (dd, 1H), 4.46 (t, 1H), 5.05 (d_{brd}, 3H), 7.18-7.23 (m, 4H), 7.53-7.63 (m, 4H), 7.8 (brd, 1H), 7.91-8.07 (m, 3H), 8.77 (s_{brd}, 2H), 9.45 (brd, 1H), 10.85 (brd, 1H). MS (APCI):1375 (M+1).

### EXAMPLE 43

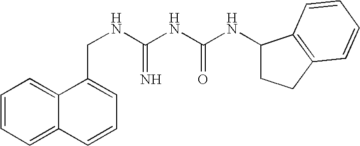

1-({[{[(2,3-Dihydro-1H-inden-1-ylamino)carbonyl]amino}(imino)methyl]amino}methyl)naphthalene, ¹HNMR (DMSO-d₆): δ2.78-3.0 (m, 2H), 3.85 (brd, 2H), 5.04 (brd, 3H), 7.11-7.29 (m, 4H), 7.50-7.64 (m, 4H), 7.90-8.04 (m, 3H), 8.75 (brd, 2H), 9.5 (brd, 1H), 10.55 (s, 1H). MS (APCI): 359 (M+1).

### EXAMPLE 44

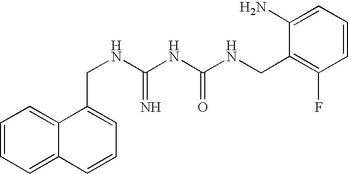

1-({[({[(2-Amino-6-fluorobenzyl)amino]carbonyl}amino)(imino)methyl]amino}methyl)naphthalene, ¹HNMR (DMSO-d₆): δ4.33 (s, 2H), 5.01 (d, 2H), 5.5 (brd, 2H), 6.6-7.29 (m, 3H), 7.49-7.63 (m, 4H), 7.89-8.03 (m, 3H), 8.15 (brd, 0.5H), 8.77 (brd, 1.5H), 9.35 (brd, 1H), 10.5 (brd, 1H). MS (APCI): 366.1 (M+1).

### EXAMPLE 45

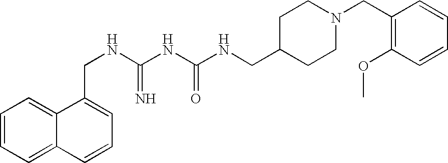

4-({[({Imino[(1-naphthylmethyl)amino]methyl}amino)carbonyl]amino}methyl)-1-(2-methoxybenzyl)piperidine, ¹HNMR (DMSO-d₆): δ1.3-1.95 (m, 5H), 2.95-3.23 (m, 4H), 3.46 (d, 2H), 3.97 (s, 2H), 3.97 (s, 3H), 4.03 (brd, 2H), 4.31 (d, 2H), 5.18 (s, 2H), 7.14 (t, 1H), 7.23 (d, 1H), 7.54-7.74 (m, 6H), 8.04-8.18 (m, 3H), 8.9 (brd, 2H), 9.6 (brd, 1H), 10.5 (brd, 1H), 11.0 (brd, 1H). MS (APCI): 460 (M+1).

### EXAMPLE 46

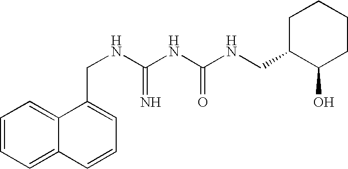

1-({[{[({[(1S,2R)-2-Hydroxycyclohexyl]methyl}amino)carbonyl]amino}(imino)methyl]amino}methyl)naphthalene, ¹HNMR (DMSO-d₆): δ0.91-1.82 (m, 9H), 2.99-3.10 (m, 2H), 3.34-3.38 (m, 1H), 3.8 (brd, 1H), 5.00 (d, 2H), 7.50-7.64 (m, 4H), 7.91-8.07 (m, 3H), 8.65 (brd, 2H), 9.35 (brd, 1H), 10.38 (brd, 1H). MS (APCI): 355.1 (M+1), (High resolution): 355.2142 (M+1).

### EXAMPLE 47

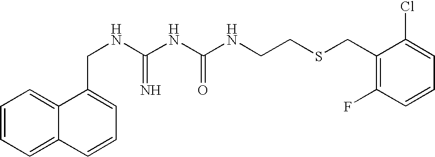

1-[10-(2-Chloro-6-fluorophenyl)-3-imino-5-oxo-9-thia-2,4,6-triazadec-1-yl]naphthalene, ¹HNMR (DMSO-d₆): δ2.65 (t, 2H), 3.3 (d, 2H), 3.87 (s, 2H), 5.01 (d, 2H), 7.2-8.04 (m, 11H), 8.7 (brd, 2H), 9.4 (brd, 1H), 10.4 (brd, 1H). MS (APCI): 444.8 (M+1).

### EXAMPLE 48

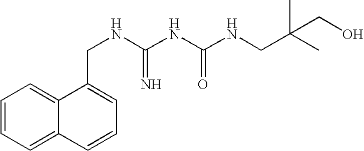

1-({[({[(3-Hydroxy-2,2-dimethylpropyl)amino]carbonyl}amino)(imino)methyl]amino}methyl)naphthalene, ¹HNMR (DMSO-d₆): δ0.79 (s, 4H), 0.93 (s, 2H), 2.99 (d, 1H), 3.08 (d, 1H), 3.14 (s, 1H), 3.3 (brd, 1H), 4.13 (s, 1H), 5.01 (d, 1H), 7.50-7.64 (m, 4H), 7.91-8.04 (m, 3H), 8.7 (brd, 2H), 9.35 (brd, 1H), 10.4 (brd, 1H). MS (APCI): 329 (M+1).

### EXAMPLE 49

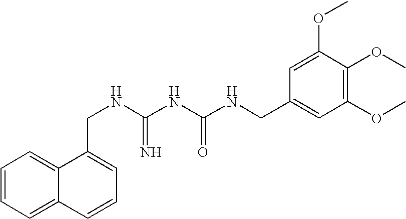

1-({[Imino({[(3,4,5-trimethoxybenzyl)amino]carbonyl}amino)methyl]amino}methyl)naphthalene, ¹HNMR (DMSO-d₆): δ3.63 (s, 3H), 3.75 (s, 6H), 4.24 (d, 2H), 5.02 (d, 2H), 6.63 (s, 2H), 7.50-7.62 (m, 4H), 7.90-8.04 (m, 3H), 8.35 (brd, 1H), 8.75 (brd, 2H), 9.4 (brd, 1H), 10.6 (brd, 1H). MS (APCI): 423 (M+1).

### EXAMPLE 50

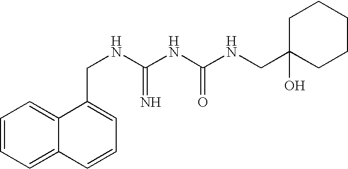

1-({[[({[(1-Hydroxycyclohexyl)methyl]amino}carbonyl)amino](imino)methyl]amino}methyl)naphthalene, ¹HNMR (DMSO-d₆): δ1.18-1.59 (m, 11H), 3.06 (d, 2H), 5.02 (d, 2H), 7.50-7.63 (m, 4H), 7.90-8.04 (m, 3H), 8.70 (brd, 2H), 9.45 (brd, 1H0, 10.55 (s, 1H). MS (APCI): 355.2 (M+1).

### EXAMPLE 51

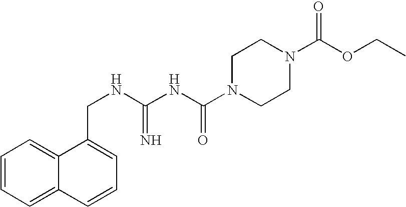

Ethyl 4-[({imino[(1-naphthylmethyl)amino]methyl}amino)carbonyl]-1-piperazine carboxylate, ¹HNMR (DMSO-d₆): δ1.17 (t, 3H), 3.40 (brd, 4H), 3.58 (brd, 4H), 4.04 (q, 2H), 5.03 (d, 2H), 7.51-7.64 (m, 4H), 7.90-8.04 (m, 3H), 8.92 (brd, 1H), 9.10 (brd, 1H), 9.73 (t_{brd}, 1H), 10.98 (s, 1H). MS (APCI): 384 (M+1), (High resolution): 384.2046 (M+1).

### EXAMPLE 52

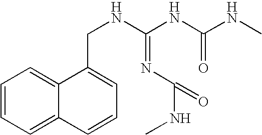

N-Methyl-N′-{(Z)-{[(methylamino)carbonyl]amino}[(1-naphthylmethyl)amino]methylidene}urea, ¹HNMR (DMSO-d₆): δ1.7 (brd, 1H), 2.79 (d, 6H), 4.98 (d, 2H), 5.21 (brd, 1H), 5.4 (brd, 1H), 7.40-7.57 (m, 4H), 7.79 (d, 1H), 7.87 (d, 1H), 8.03 (d, 1H), 9.04 (brd, 1H), 12.35 (brd, 1H). MS (APCI): 314 (M+1).

### EXAMPLE 53

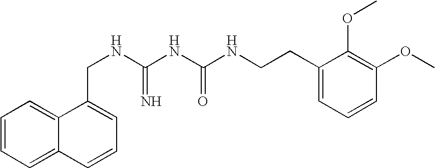

1-({[({[(2,3-Dimethoxyphenethyl)amino]carbonyl}amino)(imino)methyl]amino}methyl)naphthalene, ¹HNMR (DMSO-d₆): δ2.73 (t, 2H), 3.28 (q, 2H), 3.70 (s, 3H), 3.77 (s, 3H), 4.98 (d, 2H), 6.77 (d, 1H), 6.89-7.01 (m, 2H), 7.46-7.64 (m, 4H), 7.78 (brd, 1H), 7.93 (d, 1H), 7.99 (d, 1H), 8.01 (d, 1H), 8.73 (s_{brd}, 2H), 9.38 (brd, 1H), 10.12 (brd, 1H). MS (APCI): 407.1 (M+1).

### EXAMPLE 54

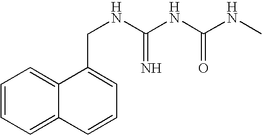

1-{[(1-Imino{[(methylamino)carbonyl]amino}methyl)amino]methyl}naphthalene, ¹HNMR (DMSO-d₆): δ2.65 (d, 3H), 4.99 (d, 2H), 7.43-7.64 (m, 5H), 7.91-8.09 (m, 3H), 8.71 (s_{brd}, 2H), 9.39 (brd, 1H), 10.33 (brd, 1H). MS (APCI): 256.9 (M+1), (high resolution): 257.1408 (M+1).

### EXAMPLE 55

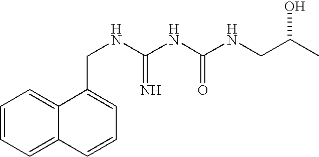

1-({[[({[(2R)-2-Hydroxypropyl]amino}carbonyl)amino](imino)methyl]amino}methyl)naphthalene, ¹HNMR (DMSO-d₆): δ1.03 (d, 3H), 2.98 (m, 1H), 3.09 (m, 1H), 3.68 (q, 1H), 4.87 (brd, 1H), 4.90 (d, 2H), 7.46-7.64 (m, 5H), 7.93 (d, 1H), 8.00 (d, 1H), 8.02 (d, 1H), 8.72 (brd, 2H), 9.42 (brd, 1H), 10.11 (brd, 1H). MS (High resolution) 301.1676 (M+1).

### EXAMPLE 56

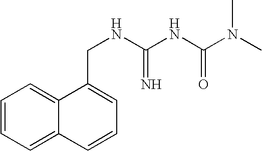

1-({[{[(Dimethylamino)carbonyl]amino}(imino)methyl]amino}methyl)naphthalene, ¹HNMR (DMSO-d₆): δ2.98 (brd, 6H), 5.00 (d, 2H), 7.49-7.65 (m, 4H), 7.91-8.03 (m, 3H), 8.84 (brd, 1H), 9.18 (brd, 1H), 9.75 (t_{brd}, 1H), 10.42 (s, 1H). MS (APCI): 271 (M+1).

### EXAMPLE 57

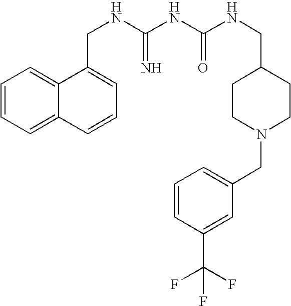

4-({[({Imino[(1-naphthylmethyl)amino]methyl}amino)carbonyl]amino}methyl)-1-[3-(trifluoromethyl)benzyl]piperidine, ¹HNMR (DMSO-d₆): δ1.54-1.83 (m, 5H), 2.55-3.07 (m, 4H), 3.30 (d, 2H), 4.38 (d, 2H), 5.05 (s, 2H), 7.51-8.08 (m, 12H), 8.83 (s, 2H), 9.54 (s, 1H), 10.77 (s, 1H), 11.15 (s, 1H). MS (APCI): 498.2 (M+1).

### EXAMPLE 58

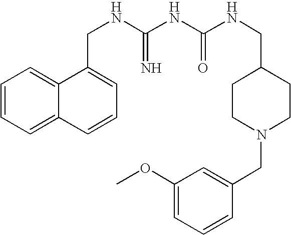

4-({[({Imino[(1-naphthylmethyl)amino]methyl}amino)carbonyl]amino}methyl)-1-(3-methoxybenzyl)piperidine, ¹HNMR (DMSO-d₆): δ1.56-1.82 (m, 5H), 2.85-3.06 (m, 4H), 5 3.25-3.29 (m, 2H), 3.78 (s, 3H), 4.21 (d, 2H), 5.05 (s, 2H), 7.00 (d, 1H), 7.31-7.36 (m, 2H), 7.51-7.62 (m, 4H), 7.91-8.13 (m, 3H), 8.84 (s brd, 2H), 9.54 (brd, 1H), 10.79 (brd, 1H), 11.03 (brd, 1H). MS (APCI): 460.2 (M+1).

### EXAMPLE 59

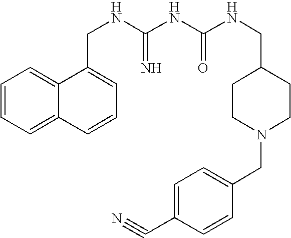

1-(4-Cyanobenzyl)-4-({[({imino[(1-naphthylmethyl)amino]methyl}amino)carbonyl]amino}methyl)piperidine, ¹HNMR (DMSO-d₆): δ1.56-1.82 (m, 5H), 2.89-3.17 (m, 4H), 3.28 (d, 2H), 4.36 (d, 2H), 5.05 (s, 2H), 7.51-7.62 (m, 4H), 7.87-8.10 (m, 8H), 8.83 (s, 2H), 9.53 (brd, 1H), 10.76 (brd, 1H), 11.31 (brd, 1H). MS (APCI): 455.3 (M+1).

### EXAMPLE 60

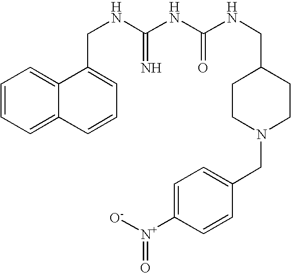

4-({[({Imino[(1-naphthylmethyl)amino]methyl}amino)carbonyl]amino}methyl)-1-(4-nitrobenzyl)piperidine, ¹HNMR (DMSO-d₆): δ1.59-1.84 (m, 5H), 2.89 (brd, 4H), 3.31 (d, 2H), 3.61 (s brd, 2H), 4.46 (d, 2H), 5.09 (s, 2H), 7.50-7.64 (m, 4H), 7.92-8.07 (m, 5H), 8.27-8.33 (m, 2H), 8.89 (s brd, 2H), 9.57 (s, 1H), 10.81 (s, 1H), 11.41 (s, 1H). MS (APCI): 475.2 (M+1).

### EXAMPLE 61

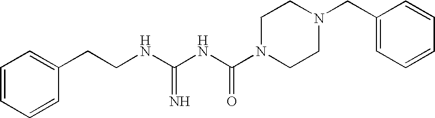

N-[(4-Benzyl-1-piperazinyl)carbonyl]-N′-phenethylguanidine, ¹HNMR (DMSO-d₆): δ2.84 (t, 2H), 3.06 (m, 2H), 3.41 (m, 2H), 3.52 (q, 2H), 3.93 (m_{brd}, 2H), 4.32 (s, 2H), 4.37 (m, 2H), 7.20-7.59 (m, 10H), 8.78 (brd, 2H), 9.29 (brd, 1H), 11.14 (brd, 1H), 11.49 (brd, 1H). MS (APCI): 366 (M+1).

### EXAMPLE 62

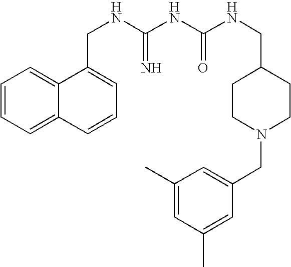

1-(3,5-Dimethylbenzyl)4-({[({imino[(1-naphthylmethyl)amino]methyl}amino)carbonyl]amino}methyl)piperidine, ¹HNMR (DMSO-d₆): δ1.5-1.82 (m, 5H), 2.28 (s, 6H), 2.8-3.05 (m, 4H), 3.29 (d, 2H), 4.14 (d, 2H), 5.04 (s, 2H), 7.07 (s, 1H), 7.21 (s, 1H), 7.51-7.6 (m, 4H), 7.91-8.01 (m, 4H), 8.82 (s, 2H), 9.53 (brd, 1H), 10.65 (brd, 1H), 10.79 (brd, 1H). MS (APCI): 458.3 (M+1).

### EXAMPLE 63

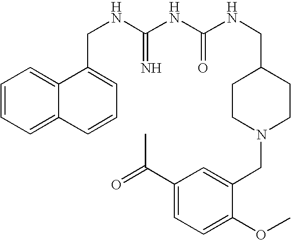

1-(5-Acetyl-2-methoxybenzyl)4({[({imino[(1-naphthylmethyl)amino]methyl}amino)carbonyl]amino}methyl)piperidine, ¹HNMR (DMSO-d₆): δ1.4-1.8 (m, 5H), 2.4 (s, 3H), 2.88-2.99 (m, 4H), 3.29 (d, 2H), 3.89 (s, 3H), 4.2 (d, 2H), 5.0 (s, 2H), 7.2 (d, 1H), 7.5-7.6 (m, 4H), 7.8-8.0 (m, 4H), 8.25 (s, 1H), 8.8 (brd, 2H), 9.5 (brd, 1H), 10.7 (brd, 1H), 10.9 (brd, 1H). MS (APCI): 502.3 (M+1).

### EXAMPLE 64

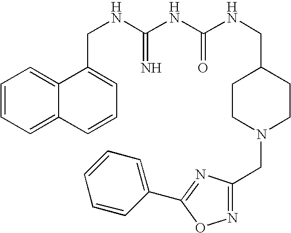

4-({[({Imino[(1-naphthylmethyl)amino]methyl}amino)carbonyl]amino}methyl)-1-[(5-phenyl-1,2,4-oxadiazol-3-yl)methyl]piperidine, ¹HNMR (DMSO-d₆): δ1.59-1.89 (m, 5H), 3.0-3.18 (m, 4H), 3.64 (d, 2H), 4.62 (s, 2H), 5.09 (s, 2H), 7.52-7.76 (m, 7H), 7.91-9.17 (m, 5H), 8.90 (s, 2H), 9.57 (brd, 1H), 10.92 (brd, 1H), 11.56 (brd, 1H). MS (APCI): 498.3 (M+1).

### EXAMPLE 65

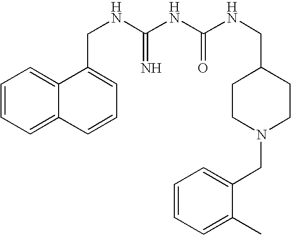

4-({[({Imino[(1-naphthylmethyl)amino]methyl}amino)carbonyl]amino}methyl)-1-(2-methylbenzyl)piperidine, ¹HNMR (DMSO-d₆): δ1.61-1.80 (m, 5H), 2.42 (s, 3H), 3.03 (m, 4H), 3.32 (m, 2H), 4.25 (d, 2H), 5.06 (s, 2H), 7.25-7.32 (m, 3H), 7.51-7.74 (m, 5H), 7.91-8.07 (m, 3H), 8.87 (s, 2H), 9.56 (brd, 1H), 10.56 (brd, 1H), 10.80 (brd, 1H). MS (APCI): 444.2 (M+1).

### EXAMPLE 66

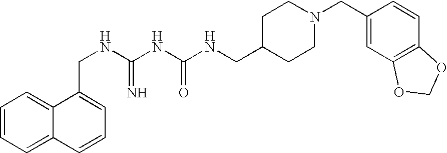

1-(1,3-Benzodioxol-5-ylmethyl)-4-({[({imino[(1-naphthylmethyl)amino]methyl}amino)carbonyl]amino}methyl)piperidine, ¹HNMR (DMSO-d₆): δ1.56-1.82 (m, 5H), 2.81-2.84 (m, 2H), 3.02 (brd, 2H), 3.27 (d, 2H), 4.15 (d, 2H), 5.04 (s, 2H), 6.07 (s, 2H), 6.96 (d, 1H), 7.03 (d, 1H), 7.30 (s, 1H), 7.51-7.62 (m, 4H), 7.92-8.03 (m, 4H), 8.83 (s, 2H), 9.54 (brd, 1H), 10.74 (brd, 1H), 10.80 (brd, 1H). MS (APCI): 474.2 (M+1).

### EXAMPLE 67

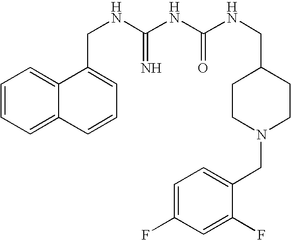

1-(2,4-Difluorobutyl)-4-({[({imino[(1-naphthylmethyl)amino]methyl}amino)carbonyl]amino}methyl)piperidine, ¹HNMR (DMSO-d₆): δ1.5-1.78 (m, 5H), 3.00-3.05 (m, 4H), 3.33 (d, 2H), 4.26 (s, 2H), 5.04 (s, 2H), 7.22-7.64 (m, 7H), 7.91-8.03 (m, 4H), 8.83 (s, 2H), 9.53 (brd, 1H), 10.78 (brd, 1H), 11.05 (brd, 1H). MS (APCI): 466.2 (M+1).

### EXAMPLE 68

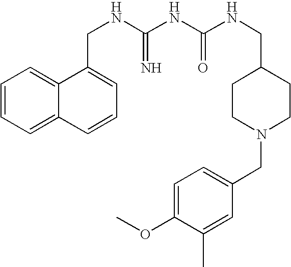

4-({[({Imino[(1-naphthylmethyl)amino]methyl}amino)carbonyl]amino}methyl)-1-(4-methoxy-3-methylbenzyl)piperidine, ¹HNMR (DMSO-d₆): δ1.49-1.81 (m, 5H), 2.15 (s, 3H), 2.81 (q, 2H), 3.00 (s_{brd}, 2H), 3.27 (d, 2H), 3.81 (s, 3H), 4.12 (d, 2H), 5.04 (s_{brd}, 2H), 6.97 (d, 1H), 7.37-7.62 (m, 6H), 7.91-8.05 (m, 3H), 8.83 (s, 2H), 9.53 (brd, 1H), 10.67 brd, 1H), 10.79 (brd, 1H). MS (APCI): 474.2 (M+1).

### EXAMPLE 69

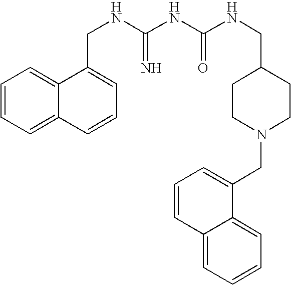

4-({[({Imino[(1-naphthylmethyl)amino]methyl}amino)carbonyl]amino}methyl)-1-(1-naphthylmethyl)piperidine, ¹HNMR (DMSO-d₆): δ1.57-1.89 (m, 5H), 2.98-3.32 (m, 4H), 3.68 (brd, 2H), 4.86 (s, 2H), 5.04 (s, 2H), 7.51-7.59 (m, 7H), 7.91-8.12 (m, 6H), 8.42 (d, 1H), 8.84 (brd, 2H), 9.54 (brd, 1H), 10.63 (brd, 1H), 10.83 (brd, 1H). MS (APCI): 480.2 (M+1).

### EXAMPLE 70

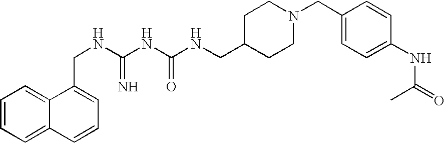

N-(4-{[4-({[({Imino[(1-naphthylmethyl)amino]methyl}amino)carbonyl]amino}methyl)-1-piperidinyl]methyl}phenyl)acetamide, ¹HNMR (DMSO-d₆): δ1.49-1.82 (m, 5H), 2.06 (s, 3H), 2.81-2.89 (m, 2H), 3.00 (brd, 2H), 3.28 (d, 2H), 4.16 (d, 2H), 5.03 (s, 2H), 7.49-7.67 (m, 8H), 7.91-8.05 (m, 4H), 8.80 (s, 2H), 9.52 (brd, 1H), 10.27 (s, 1H), 10.68 (m, 2H). MS (APCI): 487.2 (M+1).

### EXAMPLE 71

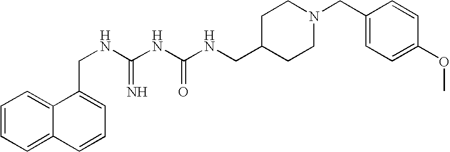

4-({[({Imino[(1-naphthylmethyl)amino]methyl}amino)carbonyl]amino}methyl)-1-(4-methoxybenzyl)piperidine, ¹HNMR (DMSO-d₆): □□1.51-1.82 (m, 5H), 2.80-2.89 (m, 2H), 3.00 (brd, 2H), 3.27 (d, 2H), 3.77 (s, 3H), 4.16 (d, 2H), 6.98 (d, 2H), 7.51-7.56 (m, 6H), 7.91-8.05 (m, 4H), 8.81 (s, 2H), 9.53 (brd, 1H), 10.74 (s, 2H). MS (APCI): 460.2 (M+1).

### EXAMPLE 72

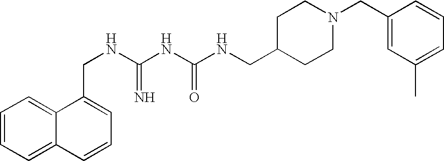

4-({[({Imino[(1-naphthylmethyl)amino]methyl}amino)carbonyl]amino}methyl)-1-(3-methylbenzyl)piperidine, ¹HNMR (DMSO-d₆): δ1.52-1.82 (m, 5H), 2.33 (s, 3H), 2.84-3.04 (m, 4H), 3.28 (d, 2H), 4.18 (d, 2H), 5.03 (s, 2H), 7.26-7.62 (m, 8H), 7.91-8.02 (m, 4H), 8.80 (s, 2H), 9.51 (s, 1H), 10.73 (s, 2H). MS (APCI): 444.2 (M+1).

### EXAMPLE 73

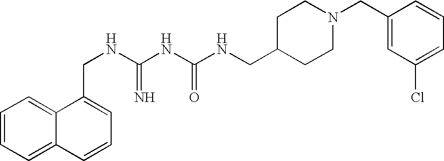

1-(3-Chlorobenzyl)-4-({[({imino[(1-naphthylmethyl)amino]methyl}amino)carbonyl]amino}methyl)piperidine, ¹HNMR (DMSO-d₆): δ1.56-1.82 (m, 5H), 2.85-3.07 (m, 4H), 3.30 (d, 2H), 4.26 (d, 2H), 5.03 (s, 2H), 7.47-7.63 (m, 7H), 7.91-8.05 (m, 4H), 8.80 (s, 2H), 9.51 (brd, 1H), 10.92 (brd, 1H), 11.05 (brd, 1H). MS (APCI): 464.3 (M+1).

### EXAMPLE 74

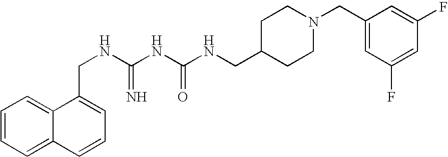

1

-(3,5-Difluorobenzyl)-4-({[({imino[(1-naphthylmethyl)amino]methyl}amino) carbonyl]amino}methyl)piperidine, ¹HNMR (DMSO-d₆): δ1.54-1.82 (m, 5H), 2.85-3.07 (m, 4H), 3.30 (d, 2H), 4.28 (d, 2H), 5.02 (s, 2H), 7.33-7.63 (m, 7H), 7.92-8.05 (m, 4H), 8.79 (s, 2H), 9.50 (brd, 1H), 10.63 (brd, 1H). MS (APCI): 466.2 (M+1).

### EXAMPLE 75

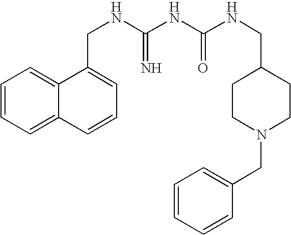

1-Benzyl-4-({[({imino[(1-naphthylmethyl)amino]methyl}amino)carbonyl]amino}methyl)piperidine, ¹HNMR (DMSO-d₆): δ1.58-1.82 (m, 5H), 2.85-2.89 (q, 2H), 3.05 (m, 2H), 3.28 (d, 2H), 4.24 (d, 2H), 5.05 (s, 2H), 7.436-7.64 (m, 9H), 7.91-8.03 (m, 3H), 8.83 (brd, 2H), 9.53 (brd, 1H), 10.74 (brd, 1H), 10.90 (brd, 1H). MS (APCI): 430.1 (M+1).

### EXAMPLE 76

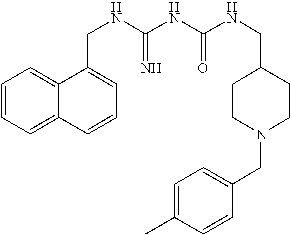

4-({[({Imino[(1-naphthylmethyl)amino]methyl}amino)carbonyl]amino}methyl)-1-(4-methylbenzyl)piperidine, ¹HNMR (DMSO-d₆): δ1.55-1.81 (m, 5H), 2.32 (s, 3H), 2.84-2.88 (m, 2H), 3.00 (m, 2H), 3.26 (d, 2H), 4.20 (d, 2H), 5.08 (s, 2H), 7.23 (d, 1H), 7.28 (d, 1H), 7.48-7.64 (m, 5H), 7.91-8.16 (m, 3H), 8.89 (s, 2H), 9.56 (brd, 1H), 10.80 (brd, 1H), 10.96 (brd, 1H). MS (APCI): 444.7 (M+1).

### EXAMPLE 77

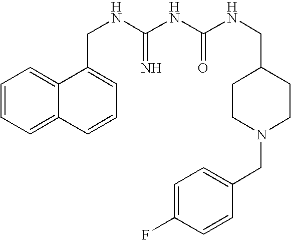

1-(4-Fluorobenzyl)-4-({[({imino[(1-naphthylmethyl)amino]methyl}amino)carbonyl]amino}methyl)piperidine, ¹HNMR (DMSO-d₆): δ1.53-1.82 (m, 5H), 2.84-3.00 (m, 4H), 3.24 (d, 2H), 4.24 (s, 2H), 5.04 (s, 2H), 7.26 (d, 1H), 7.29 (d, 1H), 7.51-7.72 (m, 6H), 7.91-8.12 (m, 3H), 8.83 (s, 2H), 9.53 (brd, 1H), 10.72 (brd, 1H), 10.94 (brd, 1H). MS APCI): 448.3 (M+1).

### EXAMPLE 78

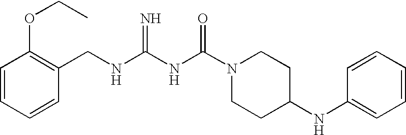

N-[(4-Anilino-1-piperidinyl)carbonyl]-N′-(2-ethoxybenzyl)guanidine, ¹HNMR (DMSO-d₆): δ1.37 (t, 5H), 1.95 (d, 2H), 3.10 (t, 2H), 3.53 (m, 1H), 3.96 (brd, 2H), 4.09 (q, 2H), 4.47 (d, 2H), 6.65-6.75 (m, 3H), 6.93-6.98 (m, 1H), 7.01-7.06 (m, 1H), 7.12-7.17 (m, 2H), 7.27-7.35 (m, 2H), 8.65 (s, 2H), 9.25 (brd, 1H), 10.08 (s, 1H). MS (APCl) 396.4 (M+1).

### EXAMPLE 79

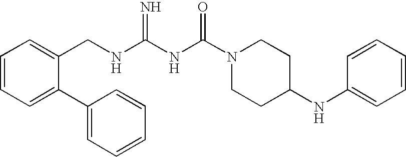

N-[(4-Anilino-1-piperidinyl)carbonyl]-N′-([1,1′-biphenyl]-2-ylmethyl)guanidine, ¹HNMR (DMSO-d₆): δ1.59 (q, 2H), 1.94 (m, 2H), 3.0 (brd, 2H), 3.66 (t, 1H), 4.28 (brd, 2H), 4.40 (d, 2H), 7.26-7.48 (m, 14H), 8.72 (brd, 1H), 8.97 (brd, 1H), 9.46 (s, 1H), 10.91 (s, 1H). MS (APCI): 428.1 (M+1).

### EXAMPLE 80

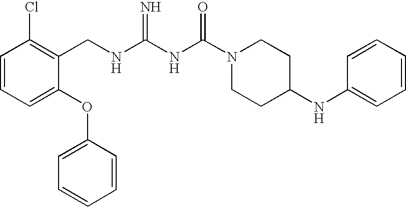

N-[(4-Anilino-1-piperidinyl)carbonyl]-N′-(2-chloro-6-phenoxybenzyl)guanidine, ¹HNMR (DMSO-d₆): δ1.56 (d, 2H), 1.92 (d, 2H), 2.93 (m, 2H), 3.62 (m, 1H), 4.25 (m, 2H), 4.63 (d, 2H), 6.82 (d, 1H), 6.85-7.43 (m, 12H), 8.93 (brd, 1H). 9.09 (brd, 1H), 9.50 (s, 1H), 10.87 (s, 1H). MS (APCI): 478.1 (M+1).

### EXAMPLE 81

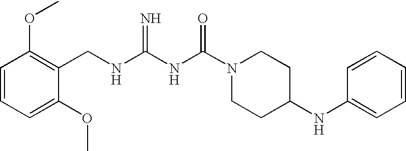

N-[(4-Anilino-1-piperidinyl)carbonyl]-N′-(2,6-dimethoxybenzyl)guanidine, ¹HNMR (CDCl₃): δ1.32 (q, 2H), 1.92 (d, 2H), 3.07 (t, 2H), 3.46-3.53 (m, 1H), 3.83 (s, 6H), 3.91 (m, 2H), 4.40 (d, 2H), 6.56 (t, 1H), 6.62 (d, 2H), 6.75 (d, 2H), 7.10 (t, 2H), 7.39 (t, 1H), 8.64 (brd, 2H), 8.76 (t, 1H), 9.75 (s, 1H). MS (APCI): 412.2 (M+l).

### EXAMPLE 82

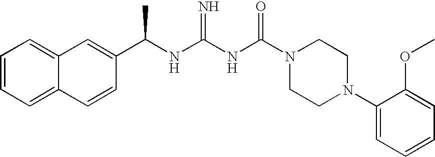

N-{[4-(2-Methoxyphenyl)-1-piperazinyl]carbonyl}-N′-[(1R)-1-(2-naphthyl) ethyl]guanidine, ¹HNMR (DMSO-d₆): δ1.56 (d, 3H), 3.10 (brd, 4H), 3.43-3.49 (m, 2H), 3.63-3.70 (m, 2H), 3.81 (s, 3H), 5.18 (q, 1H), 6.89-7.08 (m, 5H), 7.48-7.56 (m, 3H), 7.90-7.97 (m, 3H), 8.80 (brd, 1H), 9.13 (brd, 1H), 10.03 (brd, 1H), 11.02 (brd, 1H). MS (APCI): 432.2 (M+1).

### EXAMPLE 83

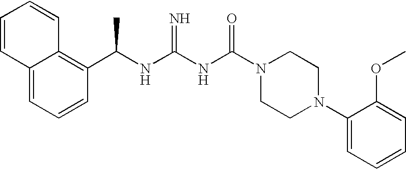

N-{[4-(2-Methoxyphenyl)-1-piperazinyl]carbonyl}-N′-[(1R)-1-(1-naphthyl)ethyl]guanidine, ¹HNMR (DMSO-d₆): δ1.60 (d, 3H), 3.23 (brd, 4H), 3.78 (s, 3H), 3.84 (brd, 4H), 5.85 (q, 1H), 6.71 (brd, 1H), 6.96 (t, 1H), 7.07 (d, 1H), 7.15 (d, 1H), 7.23 (m, 2H), 7.51-7.68 (m, 4H), 7.90 (d, 1H), 7.95 (d, 1H), 8.13 (d, 1H), 9.08 (brd, 1H), 9.21 (brd, 1H), 10.06 (d, 1H), 11.22 (s, 1H). MS (APCI): 432.1 (M+1).

### EXAMPLE 84

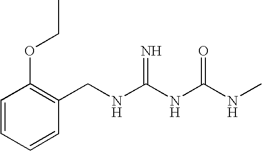

1-Ethoxy-2-{[(imino{[(methylamino)carbonyl]amino}methyl)amino]methyl}benzene, ¹HNMR (CD₃OD): δ1.30 (t, 3H), 2.60 (s, 3H), 3.97 (q, 2H), 4.33 (s, 2H), 6.77-6.88 (m, 2H), 7.13-7.21 (m, 2H). MS (APCI): 250 (M+1).

### EXAMPLE 85

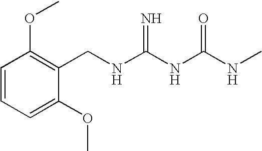

2-{[(Imino{[(methylamino)carbonyl]amino}methyl)amino]methyl}-1,3-dimethoxybenzene, ¹HNMR (CD₃OD): δ2.64 (s, 3H), 3.79 (s, 6H), 4.38 (s, 2H), 6.61 (d, 2H), 7.24 (t, 1H). MS (APCI): 267.5 (M+1).

### EXAMPLE 86

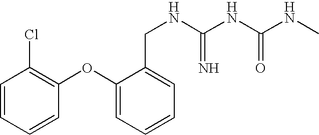

1-(2-Chlorophenoxy)-2-{[(imino{[(methylamino)carbonyl]amino}methyl)amino]methyl}benzene, ¹HNMR (DMSO-d₆): δ2.6 (s, 3H), 4.6 (s, 2H), 6.8 (s, 1H), 7.0 (s, 2H), 7.17 (s, 1H), 7.3-7.5 (m, 4H), 8.7 (brd, 2H), 9.2 (brd, 1H), 10.19 (brd, 1H). MS (APCI): 333.1 (M+1).

### EXAMPLE 87

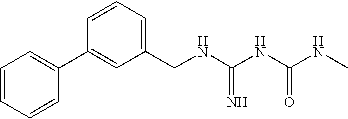

3-{[(Imino{[(methylamino)carbonyl]amino}methyl)amino]methyl}-1,1′-biphenyl, ¹HNMR (CD₃OD): δ2.28 (s, 3H), 3.94 (s, 2H), 6.83-6.95 (m, 9H). MS (APCI): 283.7 (M+1).

### EXAMPLE 88

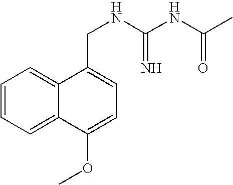

N-Acetyl-N′-[(4-methoxy-1-naphthyl)methyl]guanidine, ¹HNMR (DMSO): δ2.15(s, 3H), 3.98 (s, 3H), 4.93 (d, 2H), 6.97 (d, 1H), 7.47 (d, 1H), 7.59(t, 1H). 7.64 (t, 1H), 7.97 (d, 1H), 8.33 (d, 1H), 8.79 (bs, 1H), 9.10(bs, 1H), 12.07 (s, 1H). MS (APCI): 271.9 (M+1).

### EXAMPLE 89

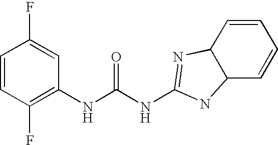

N-1H-Benzimidazol-2-yl)-N′-(2,5)difluorophenyl urea may be obtained according to conventional methods.

### Biochemical and Biological Assays

Cells and Membrane Preparation: HEK 293 cells stably expressing human 5-HT_{7B} (h5-HT_{7b}) receptors were grown in Dulbecco's Modified Eagle's Medium (DMEM; Gibco) without sodium pyruvate and containing 4.5 g/L glucose, L-glutamine/penicillin-streptomycin (Gemini), 10% fetal bovine serum and 250 mg/l of the antibiotic, G418 (Geneticin) as previously described (Jasper, J. R., Kosaka, A., To, Z. P., Chang, D. J. and Eglen, R. M. (1997) Cloning, expression and pharmacology of a truncated splice variant of the human 5-ht₇ receptor (h5-ht_{7b}). Br. J. Pharmacol. 122(1):126-132.). Cell pellets were homogenized in approximately 50 mL of homogenization buffer (buffer A) containing: 50 mM Tris (pH 7.4), 2 mM EGTA, 0.32 M sucrose, 10 μM PMSF, 1 μg/mL leupeptin, 5 μg/mL Pepstatin A, and 5 μg/mL aprotinin using an UltraTurax homogenizer (Tekmar Company, Cincinnati, Ohio) at 80% maximum setting three times for 10 sec. Cell pellets were centrifuged at 4° C. at 1,500×g for 10 min in a Beckman GS-6R centrifuge. Pellets were resuspended in buffer A, homogenized and centrifuged as described above. Pooled supernatants were transferred to centrifuge bottles and centrifuged at 4° C. at 20,000×g for 30 min in a Beckman J2-HS centrifuge. Cell pellets were resuspended in buffer A and were centrifuged at 4° C. at 20,000×g for 30 min. Cell pellets were resuspended in buffer A and stored at −70° C. in aliquots of 2.5 mg/mL total membrane protein. Total membrane protein was assessed utilizing a BCA kit (Pierce; Rockford, Ill.). Membranes containing human 5-HT₁ₐ or 5HT₂ₐ receptors expressed in CHO K1 cells were prepared as described above. Membranes bearing human D_{2S} dopamine (hD_{2S}-DA) receptors expressed in A9 L cells and human 5-HT₆ (h5-HT₆) receptors expressed in HEK-293 cells were purchased from Receptor Biology, Inc. (Beltsville, Md.) and were utilized according to the suggested guidelines provided by the manufacturer.

Radioligand Binding Assays: For 5-HT₇ saturation binding experiments, HEK-293 cell membranes expressing h5-HT₇ receptors (5-10 μg membrane protein/well) were incubated in duplicate with [³H]5-CT (approximately 0.2 nM) in binding assay buffer containing: 50 mM HEPES (pH 7.4), 0.5 mM EDTA, 10 mM MgCl₂, 10 μM pargyline to inhibit monoamine oxidase activity, and 0.1% sodium ascorbate, in a final volume of 200 μL in 96-well polypropylene plates for 2 hours at 37° C. Nonspecific binding was determined by incubating membranes with 1 μM 5-HT. All radioligand binding assays were stopped by rapid filtration onto 96-well GF/C filter plates (Packard) soaked in 0.1% polyethylenimine. Filters were washed three times with ice-cold phosphate-buffered saline (PBS) wash buffer containing 50 mM NaPO₄ (pH 7.4), 0.9% NaCl, 2 mM MgCl₂ and 0.02% NaN₃. The filters were then counted using liquid scintillation in a Packard Topcount scintillation counter.

Competition binding to the other receptor types was assayed in a similar fashion, under conditions summarized in Table 1 below.

TABLE 1
Competition Radioligand Binding Assay Conditions

| | | Nonspecific | | | Assay | |
|---|---|---|---|---|---|---|
| | [Radioligand] | binding | [Membrane] | Time/ | Volume | Binding |
| Assay | nM | defined | μg/well | Temp^{†} | (mL) | Buffer^{††} |
| H5- | [³H]5-CT | 1 μM 5-HT | 5-10 | 2 hr @ | 0.2 | A |
| HT_{7b} | 0.2-0.3 | | | 37° C. | | |
| H5- | [³H]Ketanserin | 10 nM | 10-20 | 1 hr @ | 0.2 | B |
| HT_{2b} | 0.5-1.0 | Clozapine | | 37° C. | | |
| H5- | [³H]LSD | 100 nM | 25-30 | 1 hr @ RT | 0.2 | C |
| HT₆ | 2.0-3.0 | Methiothepin | | | | |
| H5- | [³H]5-CT | 10 nM 5-CT | 5-10 | 1 hr @ RT | 0.2 | D |
| HT₁ₐ | 0.2-0.3 | | | | | |
| HD₂ₛ | [³H]Spiperone | 1 μM | 25-35 | 2 hr @ RT | 2.0 | E |
| DA | 0.08-0.15 | Haloperidol | | | | |

^{†}RT = room temperature
^{††}Buffer compositions:
A: 50 mM HEPES (pH 7.4), 0.5 mM EDTA, 10 mM MgCl₂, 10 μM pargyline, 0.1% sodium ascorbate.
B: 50 mM Tris (pH 7.4), 0.1% sodium ascorbate
C: 50 mM Tris (pH 7.4), 10 mM MgCl₂, 0.5 mM EDTA
D: 50 mM Tris (pH 7.4), 10 mM MgCl₂, 0.1% sodium ascorbate
E: 50 mM Tris (pH 7.4), 5 mM MgCl₂, 1 mM EDTA, 0.1% sodium ascorbate

Cyclic AMP Determination: The ability of various compounds to increase basal or to inhibit 5HT-stimulated cAMP formation in HEK-293 cells expressing h5-HT_{7b} receptors was assessed utilizing adenylyl cyclase flashplates custom synthesized by New England Nuclear (NEN). Cells (approximately 50,000 cells/well) were incubated with compounds in a total volume of 100 ,μl on 96-well adenylyl cyclase flashplates (NEN) for 20 minutes at room temperature with compounds to assess for agonist activity. To assess for antagonist activity, cells were incubated for 1 hr at room temperature with test compounds and then were stimulated for 20 min with 5-HT (10 nM). 100 μl of detection mix containing ¹²⁵I-cAMP was added to quench reactions according to the manufacturer's instructions. Plates were counted on a Packard TopCount after approximately two hours. Control dose-response curves to 5-HT were generated for each plate. Cyclic AMP levels were determined from standard curves generated to non-radioactive cAMP standards (10 nM-1 μM). By this method, all of the Formula I compounds acted as antagonists at 5-HT₇ receptors.

Data Analysis: Radioligand binding experiments were analyzed with Prism™ (Graphpad, San Diego, Calif.), a computer graphics and statistics program. IC₅₀ values and Hill slopes for compounds were generated by nonlinear regression using Prism™. Values for Kᵢ calculated from IC₅₀ values by the Cheng and Prussoff equation (Cheng, Y. and Prusoff, W. H., (1973), “Relationship between the inhibition constant (Kᵢ) and the concentration of inhibitor which causes 50 per cent inhibition (I₅₀) of an enzymatic reaction.” Biochemical Pharmacol. 22:3099-3108).

Biochemical Activity: Formula I compounds were assayed for binding activity vs. 5-HT₁, 5-HT_{2A}, 5-HT₆, and 5-HT₇ receptor subtypes, as well as dopamine D₂ receptors. Data are summarized in Table 2 below, where entries are blank in cases where the particular assay was not performed.

TABLE 2
Kᵢ (nM) for 5-HT and Dopamine Receptors

| Example No. | 5-HT_{1A} | 5-HT_{2A} | 5-HT₆ | 5-HT₇ | D2 |
|---|---|---|---|---|---|
| 1 | 220 | 1.0 | 2500 | 13 | 1770 |
| 2 | — | — | 3200 | 46 | >2500 |
| 3 | 1530 | 8.0 | 2720 | 100 | >2500 |
| 4 | — | — | — | 820 | — |
| 5 | 2390 | 46 | 1960 | 26 | 2010 |
| 6 | 770 | 13 | 8630 | 68 | 1050 |
| 7 | 1690 | 43 | >10000 | 1.4 | 770 |
| 8 | >4000 | 40 | >10000 | 12 | >2500 |
| 9 | — | — | — | 320 | — |
| 10 | — | — | — | 250 | >2500 |
| 11 | — | — | 910 | 25 | 920 |
| 12 | — | — | — | 180 | — |
| 13 | — | — | 3050 | 32 | 1910 |
| 14 | — | — | 1250 | 11 | 460 |
| 15 | — | 0.40 | 170 | 2.6 | 290 |
| 16 | — | — | 480 | 21 | 500 |
| 17 | — | — | 680 | 11 | 480 |
| 18 | 400 | 0.43 | 350 | 17 | 1030 |
| 19 | — | — | 930 | 110 | 200 |
| 20 | — | — | 1360 | 16 | 540 |
| 21 | 520 | 150 | >10000 | 6.0 | 900 |
| 22 | — | — | — | >4000 | — |
| 23 | 220 | 4.0 | 1930 | 9.7 | 280 |
| 24 | 2620 | 78 | 1910 | 3.2 | 820 |
| 25 | 2590 | 120 | >10000 | 7.4 | 1470 |
| 26 | — | — | 1450 | 10 | >2500 |
| 27 | — | — | 79 | 18 | >2500 |
| 28 | — | — | 3400 | 43 | 500 |
| 29 | 2690 | 170 | >10000 | 16 | >2500 |
| 30 | — | — | 1510 | 13 | 1580 |
| 31 | — | — | — | 19 | — |
| 32 | — | — | 1300 | 24 | 780 |
| 33 | — | — | 1240 | 48 | 900 |
| 34 | — | — | 7750 | 13 | 1790 |
| 35 | >4000 | 0.92 | 460 | 5.1 | 630 |
| 36 | 580 | 33 | 740 | 11 | 340 |
| 37 | — | — | — | 930 | — |
| 38 | — | — | 1180 | 14 | >2500 |
| 39 | — | — | 1180 | 120 | 400 |
| 40 | 540 | 4.1 | 590 | 24 | 1700 |
| 41 | 54 | 2.0 | 340 | 8.9 | 1070 |
| 42 | — | — | 1740 | 22 | 1250 |
| 43 | 630 | 4.8 | 900 | 22 | 2190 |
| 44 | — | — | 500 | 33 | 890 |
| 45 | 600 | 3.9 | 1550 | 21 | 500 |
| 46 | 49 | 0.60 | 500 | 9.8 | 410 |
| 47 | — | — | 530 | 120 | 860 |
| 48 | — | — | 2620 | 9.8 | 1700 |
| 49 | — | — | 1130 | 30 | 830 |
| 50 | 47 | 0.25 | 620 | 26 | 1070 |
| 51 | >4000 | 18 | >10000 | 15 | >2500 |
| 52 | — | — | — | 1010 | — |
| 53 | 170 | 0.80 | 1870 | 62 | 290 |
| 54 | 2450 | 9.6 | 2450 | 13 | >2500 |
| 55 | 1720 | 1.5 | 2320 | 10 | >2500 |
| 56 | 700 | 17 | >10000 | 94 | >2500 |
| 57 | — | 130 | — | 59 | 1570 |
| 58 | 420 | 8.1 | 1370 | 23 | 1570 |
| 59 | — | 2.6 | — | 85 | >2500 |
| 60 | 68 | 16 | 3470 | 51 | 1870 |
| 61 | 930 | 1800 | — | 490 | >2500 |
| 62 | — | 9.8 | — | 22 | 490 |
| 63 | 340 | 1.8 | 3030 | 39 | 1780 |
| 64 | — | 4.2 | — | 91 | >2500 |
| 65 | 690 | 9.5 | 4180 | 29 | 2490 |
| 66 | 430 | 4.2 | 3700 | 31 | 1580 |
| 67 | 500 | 6.4 | 2860 | 32 | >2500 |
| 68 | 1680 | 10 | 910 | 18 | 1540 |
| 69 | — | 11 | — | 27 | 720 |
| 70 | 110 | 0.55 | 2430 | 16 | 2450 |
| 71 | 600 | 1.3 | 1370 | 19 | 1470 |
| 72 | 420 | 5.3 | 880 | 15 | 880 |
| 73 | 390 | 8.7 | 750 | 21 | 830 |
| 74 | 300 | 4.1 | 1910 | 28 | 1340 |
| 75 | 340 | 4.9 | 4010 | 29 | 2300 |
| 76 | 550 | 6.8 | 3380 | 46 | 1900 |
| 77 | 1090 | 9.5 | 3350 | 19 | 1270 |
| 78 | >4000 | 170 | 6870 | 34 | >2500 |
| 79 | >4000 | 910 | 2400 | 99 | >2500 |
| 80 | 3650 | 930 | 2300 | 41 | >2500 |
| 81 | 2940 | 180 | 4700 | 66 | 1920 |
| 82 | — | — | — | 1080 | — |
| 83 | — | — | — | 750 | — |
| 84 | >4000 | 400 | 8660 | 49 | >2500 |
| 85 | 310 | 280 | 6340 | 12 | >2500 |
| 86 | 920 | 290 | 580 | 6.3 | >2500 |
| 87 | >400 | 1020 | 3360 | 80 | >2500 |
| 88 | — | — | — | 1225 | — |
| 89 | — | — | — | 2700 | — |

Biological Activity: The biological activity of the inventive compounds is determined by assays that have been devised to serve as animal models for various human medical conditions. Many such assays are known to skilled practitioners. Useful assays include: the prokinetic assay, which is an in vivo method of determining the extent the test compound affects the rate of gastric emptying of a test meal in rats; the anxiolytic behavior assay, which measures the extent to which the test compound can ameliorate the symptoms of natural anxiety in mice when exposed to a novel, brightly lighted environment; the withdrawal anxiety assay, which measures the extent to which the test compound can ameliorate the symptoms in mice caused by withdrawal from addictive substances by measuring the extent the drug affects the anxiety that occurs in mice after chronically treating with an addictive substance and then abruptly ceasing the treatments; and the cognitive enhancement assay, which measures the extent the test compound can alleviate the cognitive deficit induced in rats by administration of atropine to the rats. These assays are described in U.S. Pat. No. 5,763,468, the disclosure of which is hereby incorporated herein by reference.

While the invention has been described in terms of preferred embodiments and specific examples, those skilled in the art will recognize through routine experimentation that various changes and modifications can be made without departing from the spirit and scope of the invention. Thus, the invention should be understood as not being limited by the foregoing detailed description, but as being defined by the appended claims and their equivalents.

We claim:

1. A 5-HT₇ receptor antagonist compound having the formula: 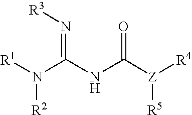
wherein:
Z is N, O or CH;
R¹ is H or lower alkyl;
R² is alkyl, cycloalkyl, arylalkyl or heteroarylalkyl, wherein the alkyl, cycloalkyl, aryl and heteroaryl moieties thereof may be substituted or unsubstituted; or
R¹ and R² together with the nitrogen to which they are bound form a 5- or 6-membered ring, which may be substituted or unsubstituted;
R³ is H, lower alkyl or lower alkylaminocarbonyl;
R⁴ is H, alkyl, alkenyl, arylalkyl, heteroarylalkyl, heterocycloalkylalkyl, arylalkenyl, heteroarylalkenyl, cycloalkyl, cycloalkenyl, heterocycloalkyl, aryl or heteroaryl, wherein the alkyl, alkenyl, cycloalkyl, cycloalkenyl, heterocycloalkyl, aryl and heteroaryl moieties thereof may be substituted or unsubstituted; or
R⁵ is absent (when Z is O) or is H or lower alkyl; or
R⁴ and R⁵ together with Z form a 5- or 6-membered ring, which may be substituted or unsubstituted;
and pharmaceutically acceptable salts, solvates, active metabolites, or prodrugs thereof.

2. A compound according to claim 1, wherein R¹, R³ and R⁵ are H.

3. A compound according to claim 1, having the formula:

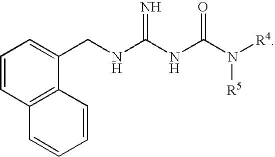

4. A compound according to claim 1, wherein Z is N.

5. A compound according to claim 1, wherein Z is CH.

6. A compound according to claim 1, wherein Z is O.

7. A compound according to claim 1, wherein Z, R⁴ and R⁵ form a 5- or 6membered ring, having formula: 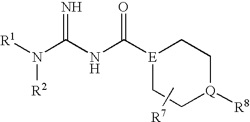
wherein:
R¹ and R² are as defined in claim 1;
E is N or CH;
Q is N or CH;
R⁷ and R⁸ are independently selected from H, substituted or unsubstituted lower alkyl, cycloalkenyl, heteroaryl, heterocycloalkyl, arylalkyl, arylalkenyl, heteroarylalkyl, heterocycloalkylalkyl, aryl, cyano, amino, alkylamino, arylamino, dialkylamino, keto, hydroxyalkyl, hydroxyl, alkoxy, alkylenedioxy, haloalkoxy, aryloxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, arylcarbonyl, alkylthio or arylthio groups, wherein the alkyl, alkenyl, cycloalkyl, heterocycloalkyl, aryl or heteroaryl moieties thereof may be substituted by one or more substituents independently selected from alkyl, haloalkyl, aminoalkyl, alkylhydroxyl, aryl, nitro, cyano, amino, alkylamino, arylamino, dialkylamino, mercapto halo, hydroxyl, alkoxy, alkylenedioxy, haloalkoxy, aryloxy, alkylcarbonylamino, alkylaminocarbonyl, alkylcarbonyl, aminocarbonyl, alkoxycarbonyl, arylcarbonyl, alkylthio or arylthio groups or may be substituted by a spiro, fused or spiro-fused cycloalkyl or heterocycloalkyl group which may be unsubstituted or substituted by alkyl, haloalkyl, aminoalkyl, arylalkyl, aryl, heteroaryl, nitro, cyano, amino, alkylamino, arylamino, dialkylamino, halo, hydroxyl, alkylhydroxyl, aryloxy, alkylcarbonylamino, alkylaminocarbonyl, alkylcarbonyl, aminocarbonyl, alkoxycarbonyl, arylcarbonyl or aryloxycarbonyl, wherein the aryl moieties of any of the above substituents are optionally substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups; or, when Q is CH, R⁸ may also be selected from halo, nitro, or mercapto; and pharmaceutically acceptable salts, solvates, active metabolites, or prodrugs thereof.

8. A compound according to claim 7, wherein E and Q are both N, having formula: 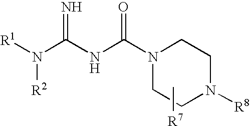
wherein R¹ are R² are as defined in claim 9;
R⁷ is selected from H, substituted or unsubstituted lower alkyl, amino, alkylamino, dialkylamino, halo, keto, hydroxyalkyl, hydroxyl, alkoxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, wherein the alkyl or aryl moieties thereof may be substituted by one or more substituents independently selected from alkyl, haloalkyl, aminoalkyl, alkylhydroxyl, nitro, cyano, amino, alkylamino, dialkylamino, halo, hydroxyl, alkoxy, alkylenedioxy, haloalkoxy, alkylcarbonylamino, alkylaminocarbonyl, alkylcarbonyl, aminocarbonyl or alkoxycarbonyl groups;
R⁸ is selected from H, substituted or unsubstituted lower alkyl, cycloalkenyl, heteroaryl, heterocycloalkyl, arylalkyl, arylalkenyl, heteroarylalkyl, heterocycloalkylalkyl, aryl, cyano, amino, alkylamino, arylamino, dialkylamino, hydroxyalkyl, hydroxyl, alkoxy, haloalkoxy, aryloxy, alkylcarbonylamino, alkylaminocarbonyl, alkylcarbonyl, alkoxycarbonyl, aryloxycarbonyl, aminocarbonyl, haloalkyl, aminoalkyl, alkylhydroxyl alkoxycarbonyl, arylcarbonyl, alkylthio or arylthio groups or may be substituted by a spiro, fused or spiro-fused cycloalkyl or heterocycloalkyl group which may be unsubstituted or substituted by alkyl, haloalkyl, aminoalkyl, arylalkyl, aryl, heteroaryl, nitro, cyano, amino, alkylamino, arylamino, dialkylamino, halo, hydroxyl, alkylhydroxyl, aryloxy, alkylcarbonylamino, alkylaminocarbonyl, alkylcarbonyl, aminocarbonyl, alkoxycarbonyl, arylcarbonyl or aryloxycarbonyl, wherein the alkyl, alkenyl, cycloalkyl, heterocycloalkyl, aryl or heteroaryl moieties thereof may be substituted by one or more substituents independently selected from alkyl, haloalkyl, aminoalkyl, alkylhydroxyl, aryl, nitro, cyano, amino, alkylamino, arylamino, dialkylamino, halo, hydroxyl, alkoxy, alkylenedioxy, haloalkoxy, aryloxy, alkylcarbonylamino, alkylaminocarbonyl, alkylcarbonyl, aminocarbonyl, alkoxycarbonyl, arylcarbonyl, mercapto, alkylthio or arylthio groups, wherein the aryl moieties of any of the above substituents are optionally substituted by alkyl, haloalkyl, nitro, cyano, amino, alkylamino, arylamino, dialkylamino, halo, hydroxyl, alkoxy, haloalkoxy, aryloxy, alkylcarbonylamino, alkylaminocarbonyl, alkylenedioxy, alkylcarbonyl, aminocarbonyl, alkoxycarbonyl, arylcarbonyl, mercapto, alkylthio or arylthio groups;
and pharmaceutically acceptable salts, solvates, active metabolites, or prodrugs thereof.

9. A compound according to claim 8, wherein R¹ is H.

10. A method of preparing a compound according to claim 1 comprising:
1. treating a compound having the formula: 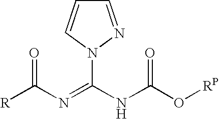
with (RCO)₂O, wherein R^{P} is substituted or unsubstituted alkyl, aryl or arylalkyl, R is CHR⁴R⁵ or substituted or unsubstituted alkyl, aryl, alkoxy, aryloxy, arylalkyl or arylalkoxy;
2. treating the product formed in step 1 with HNR¹R²; and
3. forming said compound by treating with acid.

11. A method according to claim 10, further comprising the step of treating the product formed in step 2 with HNR⁴R⁵.

12. A method according to claim 10, further comprising the step of treating the product formed in step 2 with R⁴OH.

13. A method of preparing a compound according to claim 1 comprising:
1. treating a 1-H-pyrazole-1(N,N′-bis(nitrogen-protected) carboxamidine having the formula: 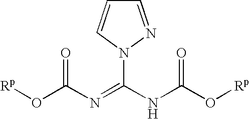
with NR¹R², wherein R^{P} is substituted or unsubstituted alkyl, aryl or arylalkyl;
2. treating the product formed in step 1 with ZR⁴R⁵; and
3. forming said compound by treating with acid.

14. An intermediate compound of formula: 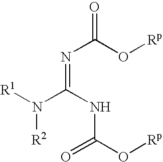
or a pharmaceutically acceptable salt thereof, wherein:
R¹ is H or lower alkyl;
R² is alkyl, cycloalkyl, arylalkyl or heteroarylalkyl, wherein the alkyl, cycloalkyl, aryl and heteroaryl moieties thereof may be substituted or unsubstituted; or
R¹ and R² together with the nitrogen to which they are bound form a 5- or 6-membered ring, which may be substituted or unsubstituted; and
R^{p} is alkyl, aryl or arylalkyl.

15. An intermediate or a pharmaceutically acceptable salt thereof according to claim 14, having formula:

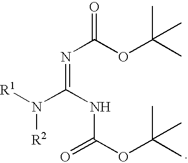

16. An intermediate or a pharmaceutically acceptable salt thereof according to claim 14, selected from the group consisting of:

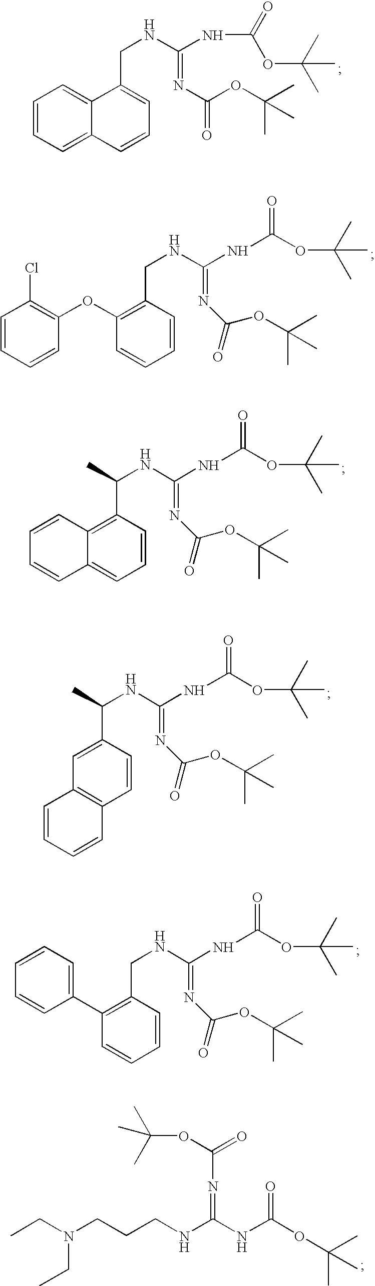

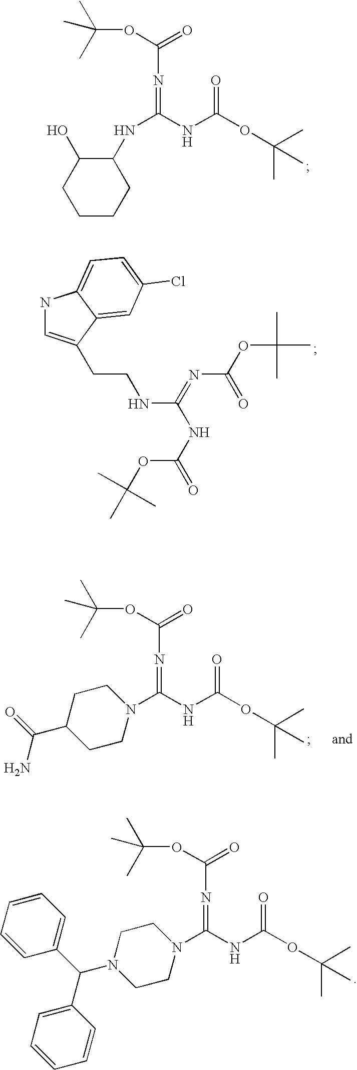

17. A pharmaceutical composition comprising an effective amount of a compound according to claim 1, or a pharmaceutically acceptable salt, solvate, active metabolite, or prodrug thereof.

18. A pharmaceutical composition comprising an effective amount of a compound according to claim 3, or a pharmaceutically acceptable salt, solvate, active metabolite, or prodrug thereof.

19. A pharmaceutical composition comprising an effective amount of a compound according to claim 8, or a pharmaceutically acceptable salt, solvate, active metabolite, or prodrug thereof.

20. A method of treatment of pain in a patient in need thereof comprising administering to said patient a pharmaceutical composition comprising an effective amount of a compound according to claim 1, or a pharmaceutically acceptable salt, solvate, active metabolite, or prodrug thereof.

21. A method of treatment of schizophrenia in a patient in need thereof comprising administering to said patient a pharmaceutical composition comprising an effective amount of a compound according to claim 1, or a pharmaceutically acceptable salt, solvate, active metabolite, or prodrug thereof.

22. A method of treatment of depression in a patient in need thereof comprising administering to said patient a pharmaceutical composition comprising an effective amount of a compound according to claim 1, or a pharmaceutically acceptable salt, solvate, active metabolite, or prodrug thereof.

23. A method of treatment of sleep disorders in a patient in need thereof comprising administering to said patient a pharmaceutical composition comprising an effective amount of a compound according to claim 1, or a pharmaceutically acceptable salt, solvate, active metabolite, or prodrug thereof.

24. A 5-HT₇ receptor antagonist compound selected from: 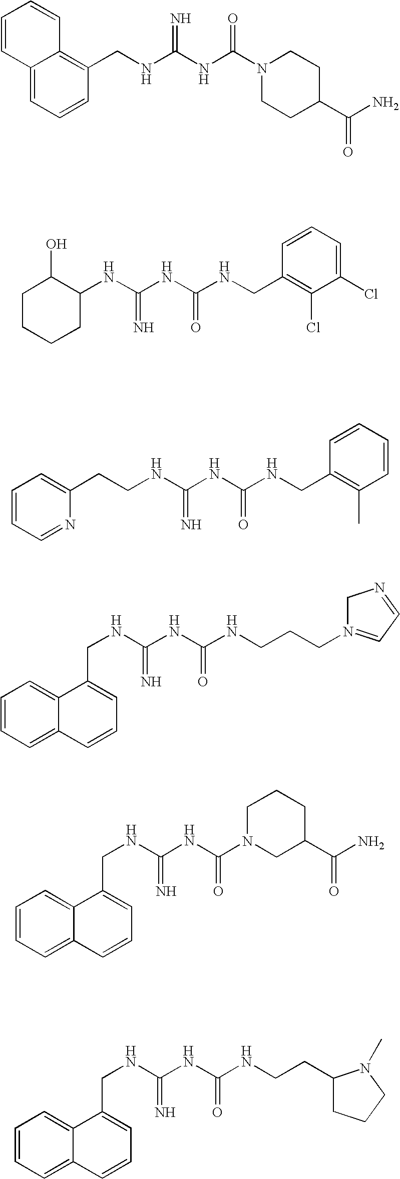 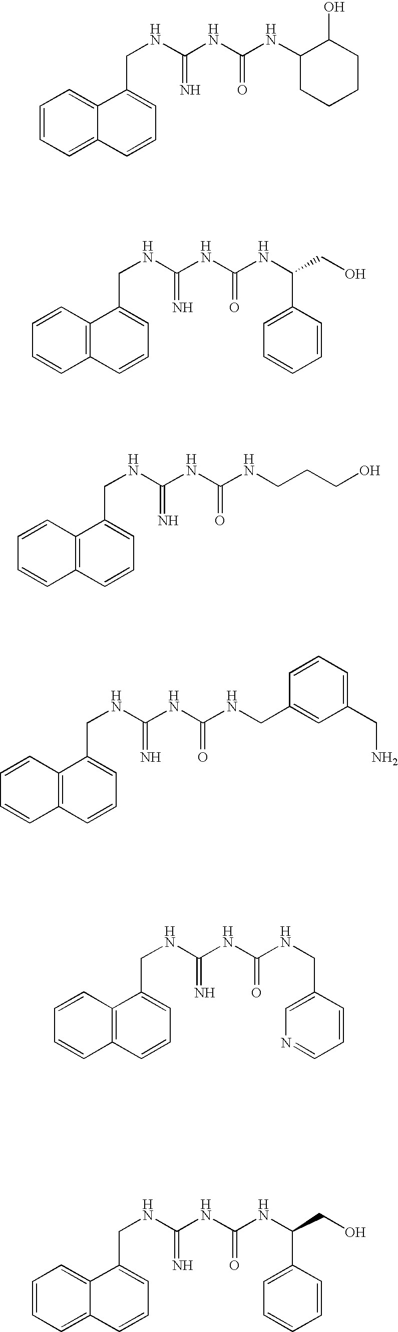 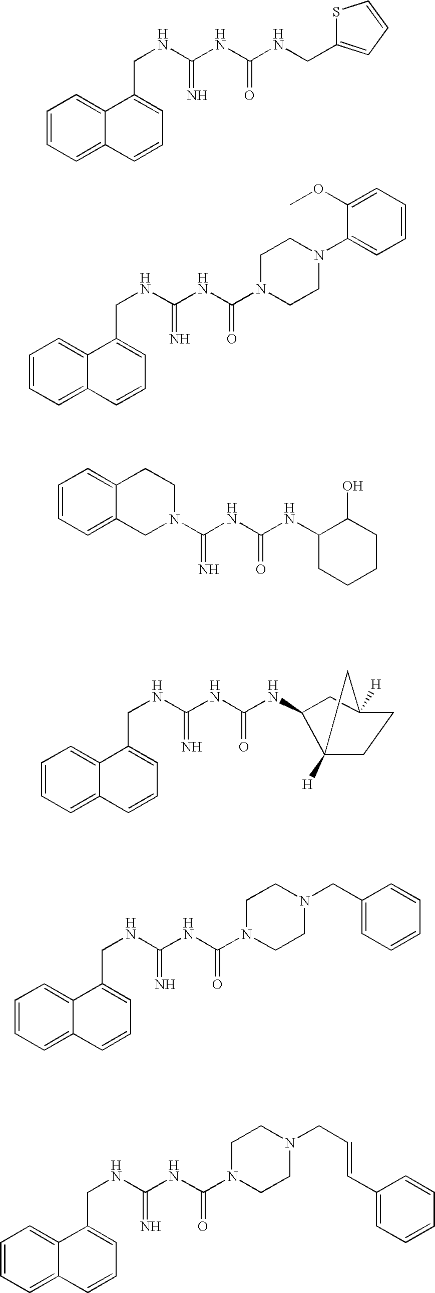 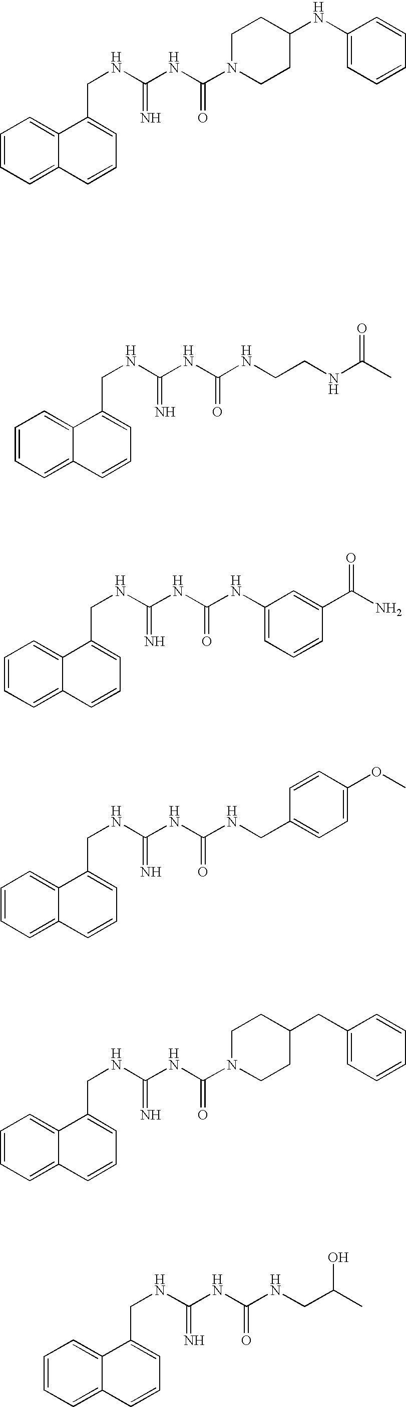 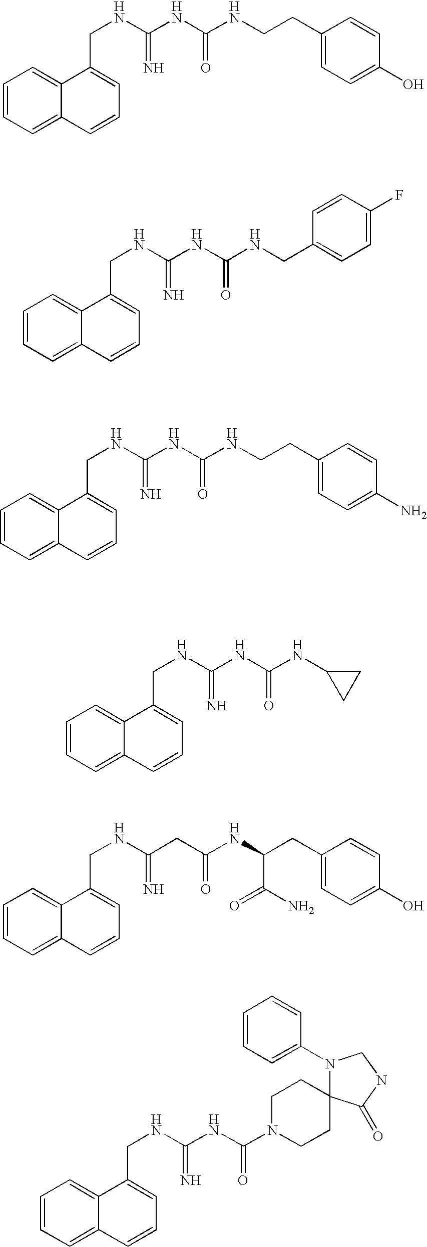 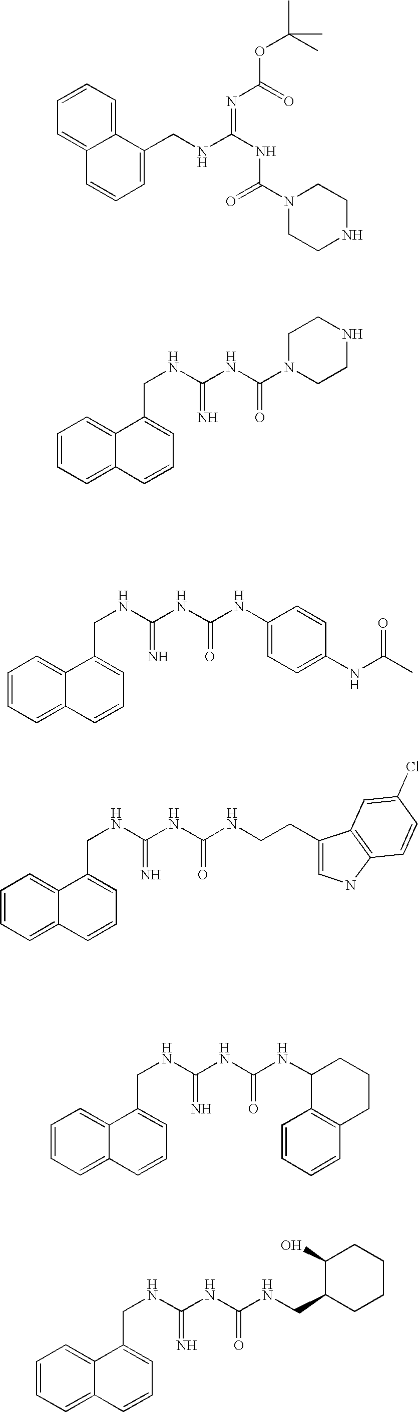 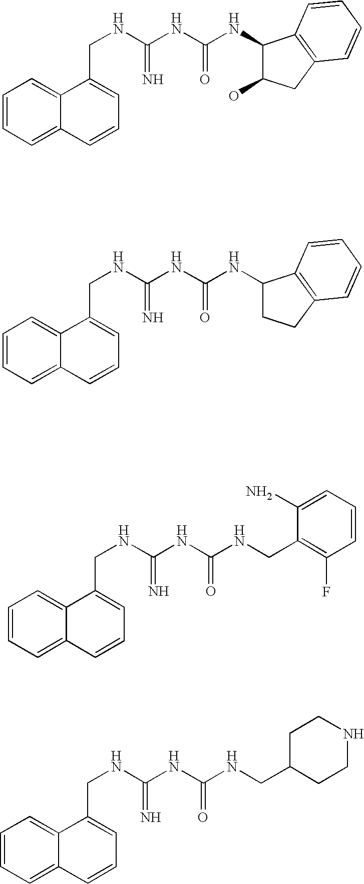 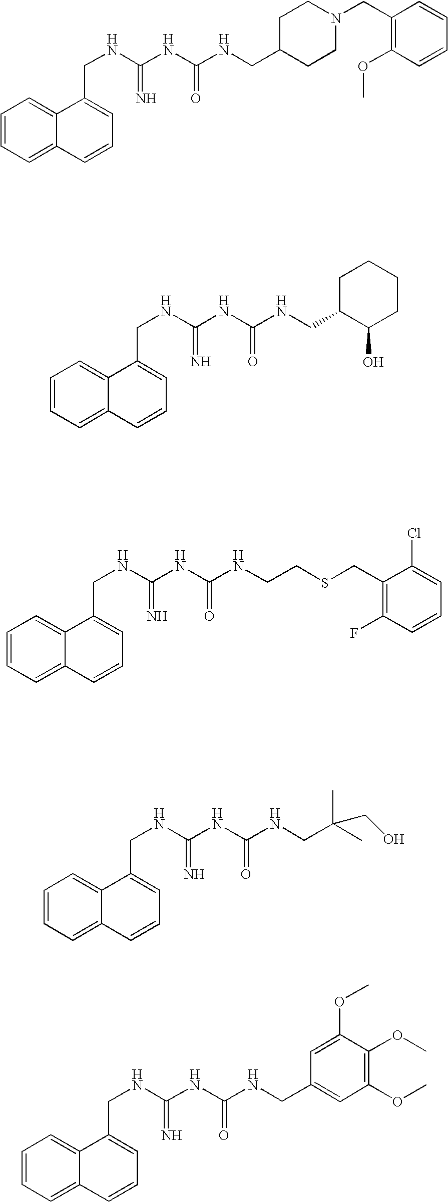 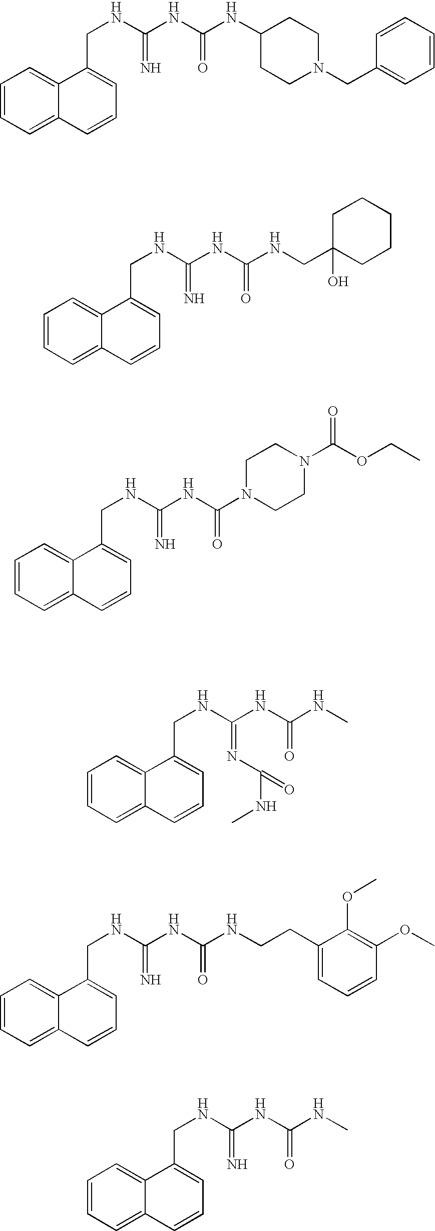 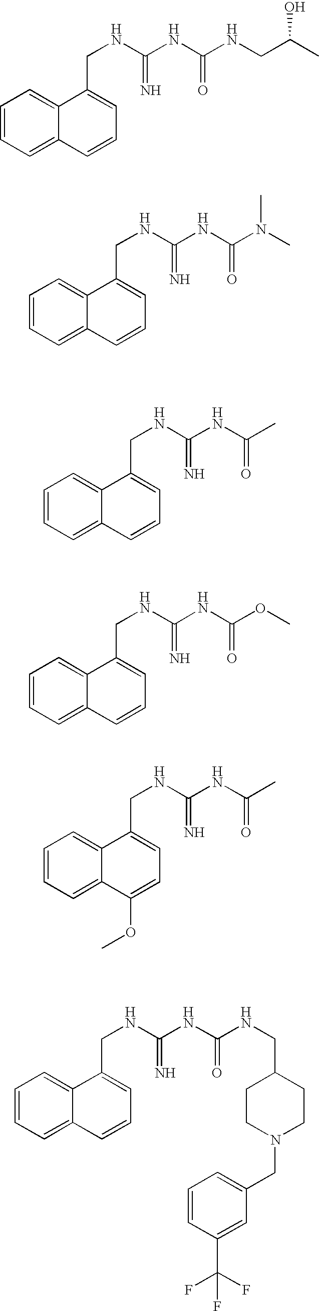 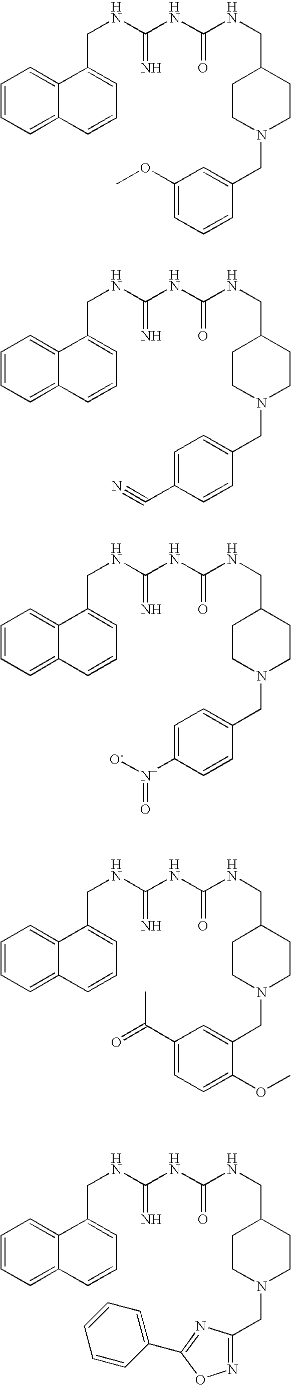 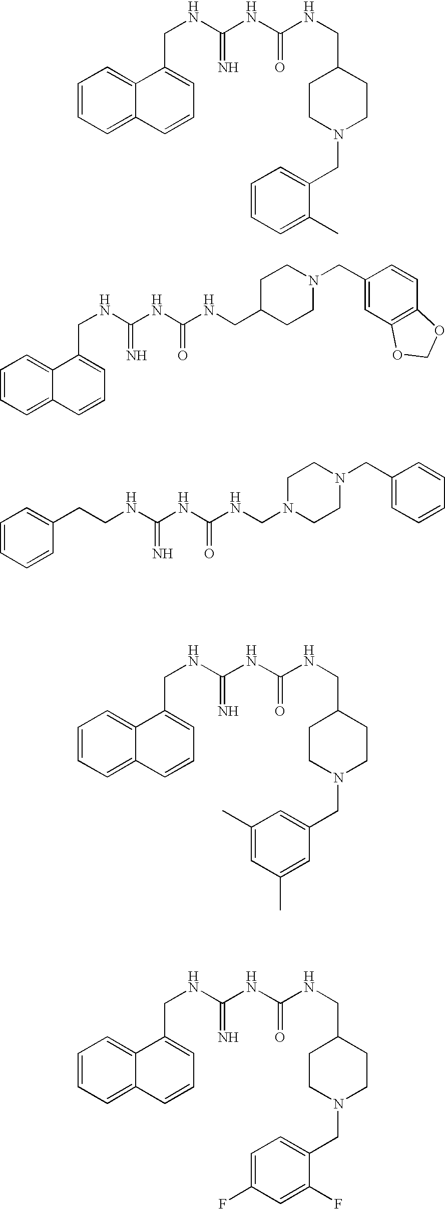 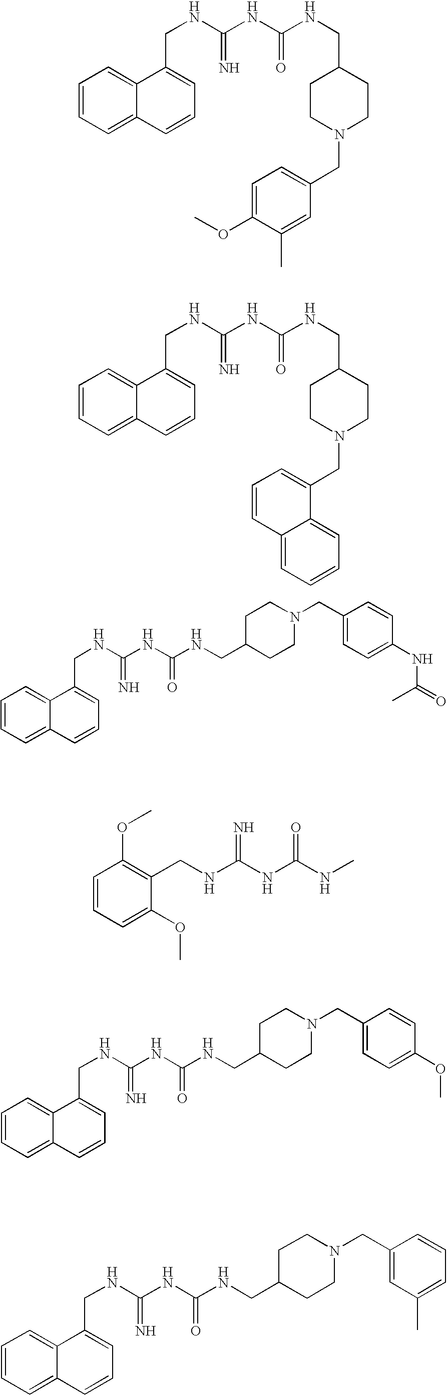 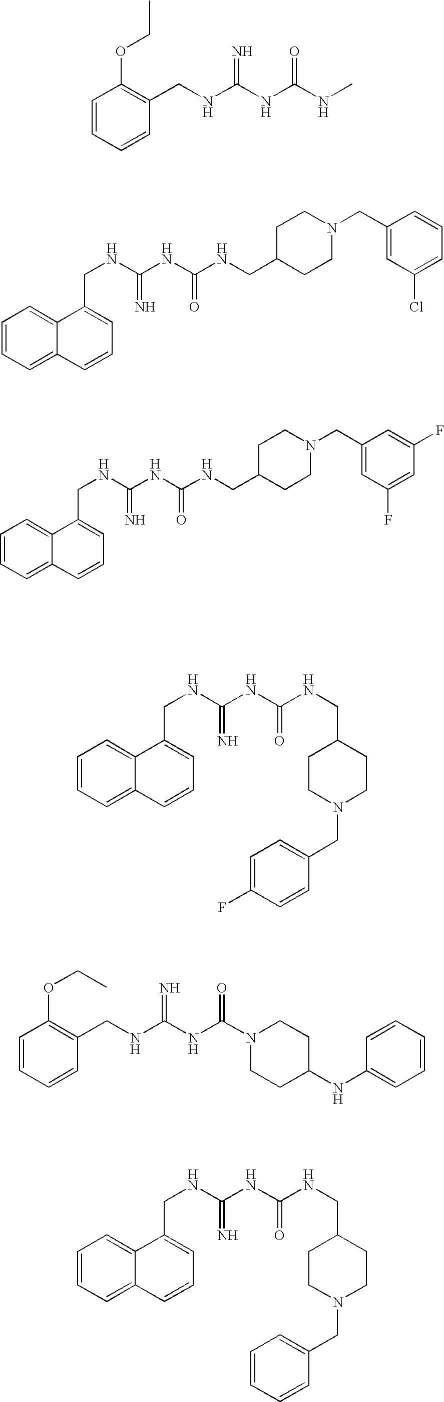 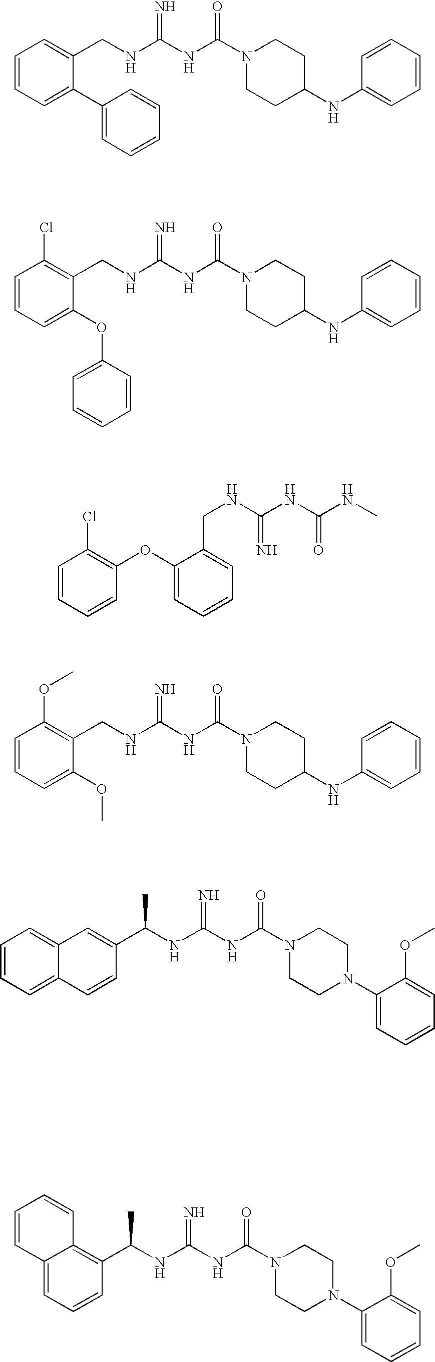 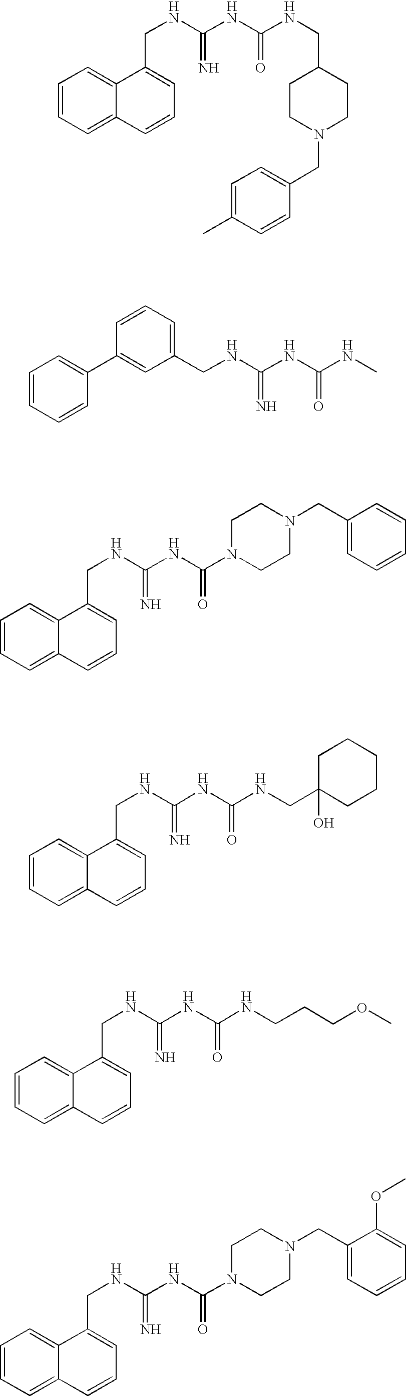
or pharmaceutically acceptable salts, solvates, active metabolites, or prodrugs thereof.

25. A compound according to claim 24 having the formula: 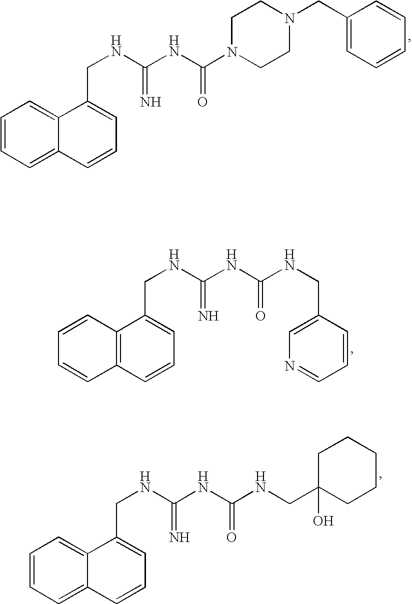
or pharmaceutically acceptable salts, solvates, active metabolites, or prodrugs thereof.

26. A pharmaceutical composition comprising an effective amount of a compound having the formula: 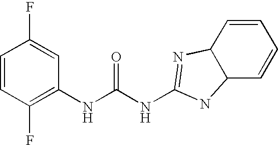
or a pharmaceutically acceptable salt, solvate, active metabolite, or prodrug thereof and a pharmaceutically acceptable carrier.

27. A method of treatment of pain, schizophrenia, depression or sleep disorders in a patient in need thereof comprising administering to said patient a pharmaceutical composition according to claim 26.
